(19) United States
(12) Patent Application Publication
(10) Pub. No.: US 2023/0010255 A1
(43) Pub. Date: Jan. 12, 2023

(54) CAR T CELLS THAT TARGET ASPERGILLUS-ASSOCIATED ANTIGENS

(71) Applicant: Atara Biotherapeutics, Inc., South San Francisco, CA (US)

(72) Inventor: Blake Tolu Aftab, South San Francisco, CA (US)

(21) Appl. No.: 17/783,469

(22) PCT Filed: Dec. 7, 2020
(86) PCT No.: PCT/US2020/063582

§ 371 Date: Jun. 8, 2022

Related U.S. Application Data
(60) Provisional application No. 62/945,380, filed on Dec. 9, 2019.

(51) Int. Cl. C07K 16/14; A61K 35/17; A61P 31/10; C07K 14/725; C07K 14/705

(52) U.S. Cl. CPC C07K 16/14; A61K 35/17; A61P 31/10; C07K 14/7051; C07K 14/70517; C07K 14/70521; A61K 2039/5156

(57) ABSTRACT

Provided herein are compositions and methods for targeted treatment of Aspergillus-associated diseases and disorders in mammals, such as diseases and disorders associated with Aspergillus infection.

### RELATED APPLICATIONS

This application claims the benefit of priority to U.S. Provisional Pat. Application serial number 62/945,380, filed Dec. 9, 2019, which is incorporated by reference in its entirety.

### BACKGROUND

Some species of Aspergillus are known to be the causative agent of a wide range of diseases. For example, Aspergillus fumigatus is associated with invasive and non-invasive pulmonary infections that are associated with a high rate of mortality. Immunosuppressed patients in particular are at high risk of developing invasive pulmonary aspergillosis, a disease characterized by a progressive necrotizing pneumonitis which, if untreated, can disseminate to other organs. What is more, patients with impaired lung function, such as those with cystic fibrosis, are at high risk of developing a chronic non-invasive infection of the airways that is associated with declining lung function, frequent hospitalizations and a high rate of mortality. Current therapeutics for both manifestations of pulmonary aspergillosis have been disappointing due to their inability to eradicate non-invasive airway infections and their poor response rate in patients with invasive infections. As such, there is a need for novel therapeutic modalities useful for the prevention or treatment of Aspergillus-associated diseases and disorders.

### SUMMARY

The present invention is based, at least in part, on the discovery that antigens expressed by Aspergillus can be used as targets for chimeric antigen receptor (CAR) -expressing T cells for the treatment of Aspergillus-associated diseases and disorders. In some aspects, provided herein are immune cells that express a CAR polypeptide that targets an Aspergillus-associated antigen. In some embodiments, the CARs disclosed herein comprise an Aspergillus antigen-targeting domain (e.g., such as a targeting domain that binds galactofuranose-containing targets), a transmembrane domain, and an intracellular signaling domain. In certain preferred embodiments, the Aspergillus antigen-binding domain targets a wildtype and/or mutant Aspergillus antigen. Preferably, the targeting domain comprises any galactofuranose-containing antigenic entity, including, but not limited to polysaccharides such as GalF2, GalF3, or GalF4; most preferably GalF4. In some embodiments, the Aspergillus antigen is an Aspergillus fumigatus or Aspergillus niger antigen, preferably an antigen that is expressed in the cell wall of Aspergillus fumigatus (e.g., the cell wall of conidia or the cell wall of hyphae). In some embodiments, the Aspergillus antigen-binding domain is a single chain Fv antibody fragment (scFv).

In some embodiments, the transmembrane domain of the CARs disclosed herein comprise at least one transmembrane domain of any of CD28, 41BB, mutants thereof, or any combination thereof. In some preferred embodiments, the intracellular signaling domain of the CARs disclosed herein comprise at least one signaling domain of any one of the polypeptides CD8, CD3ζ, CD3δ, CD3γ, CD3ε, CD32, DAP10, DAP12, CD79a, CD79b, CD28, CD3C, CD4, b2c, 41BB, ICOS, CD27, CD28δ, CD80, NKp30, OX40, FcγRI-γ, FcyRIII-y, FcεRI-β, FcεRI-γ, mutants thereof, or any combinations thereof. In some embodiments, the CARs disclosed herein further comprise at least one co-stimulatory signaling region, such as a co-stimulatory signaling region comprising a signaling domain of any one of the polypeptides CD8, CD3ζ, CD3δ, CD3γ, CD3ε, CD32, DAP10, DAP12, CD79a, CD79b, CD28, CD3C, CD4, b2c, 41BB, ICOS, CD27, CD28δ, CD80, NKp30, OX40, FcγRI-γ, FcyRIII-γ, FcεRI-β, FcεRI-γ, mutants thereof, or any combinations thereof. In certain preferred embodiments, the CARs disclosed herein comprise one or more co-stimulatory regions comprising a mutant CD28 co-stimulatory domain comprising one or more mutations in any of the YMNM, PRRP or PYAP subdomains therein. In other preferred embodiments, the CARs disclosed herein comprise one or more co-stimulatory regions comprising a mutant CD3ζ co-stimulatory domain comprising one or more mutations in any of the C-terminal immunoreceptor tyrosine-based activation motifs (ITAMs) therein. The CARs disclosed herein may comprise a hinge region.

In some embodiments, the CAR polypeptide contains an incomplete endodomain. For example, the CAR polypeptide may contain either an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3ζ signaling domain but does not contain a co-stimulatory signaling region (CSR). In other embodiments, the CAR polypeptide contains the cytoplasmic domain of CD28, 41BB, or mutants of or a combination thereof, but does not contain a CD3ζ signaling domain (SD).

In certain aspects, also provided herein are bi-specific CAR T cells, said cells expressing a first CAR polypeptide comprising a targeting domain that selectively binds an Aspergillus antigen (e.g., any galactofuranose-containing antigen, GalF4 antigen, or the like) and a second CAR polypeptide comprising a targeting domain that selectively binds to another different antigen.

In some aspects, disclosed herein are isolated nucleic acids encoding the disclosed CAR polypeptides, as well as nucleic acid vectors containing said isolated nucleic acids operably linked to an expression control sequence. Additionally, disclosed herein are cells transfected with these vectors, or that otherwise comprise the disclosed nucleic acids, or cells that express the herein disclosed CAR polypeptides. Without intending to be an exhaustive list, the cell may be an immune effector cell such as an alpha-beta T cell, a gamma-delta T cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, or a regulatory T cell. The cell expressing the herein described CAR polypeptides may also be a pluripotent stem cell, such as an induced pluripotent stem cell (iPSC). In some embodiments, the cell exhibits an antifungal immunity (e.g., mounts an immune response against a fungal entity such as Aspergillus) when the antigen-binding domain of the CAR binds to an Aspergillus-associated antigen. In certain aspects, cells expressing the CAR polypeptides of the present invention may be sensitized to one or more vial antigens. Such viral-sensitized cells may be cytotoxic T cells sensitized to one of more viral antigens selected from the group of EBV-associated antigens, CMV-associated antigens, BKV-associated antigens, and JCVassociated antigens and, as such, will comprise native T cell receptors that recognize and bind to such viral antigens. The cells of the present invention may be either autologous or allogeneic to a patient to whom they are administered.

In some aspects, provided herein are methods of preventing or treating an Aspergillus-associated disease or disorder in a mammal in need thereof comprising administering to the mammal an effective amount of an adoptive immunotherapy composition comprising CAR-expressing cells as disclosed herein. In some embodiments, the Aspergillus-associated disease or disorder to be prevented or treated is pulmonary aspergillosis, allergic bronchopulmonary aspergillosis, aspergilloma, chronic pulmonary aspergilloma, severe asthma with Aspergillus sensitization, chronic cavitary pulmonary aspergillosis, or chronic fibrosing pulmonary aspergillosis.

### DETAILED DESCRIPTION

### Details

As disclosed herein, the present invention relates, at least in part, to immune cells which recombinantly express a chimeric antigen receptor (CAR) that targets an Aspergillus-associated antigen (an “ASP” antigen). The infectious/invasive life cycle of Aspergillus begins with the production of asexual spores, referred to as conidia, which are dispersed into the air and deposited in the bronchioles or alveolar spaces via inhalation. Conidia that evade macrophage killing and colonize the respiratory tract (such as in immunosuppressed and at-risk patient populations) germinate, resulting in the growth of filamentous hyphae and invasion into the host endothelium; eventually disseminating upon reaching the bloodstream.

In a preferred embodiment, the ASP antigen is an antigen expressed in the cell wall (e.g., the cell wall of conidia or hyphae) of Aspergillus fumigatus, Aspergillus flavus, Aspergillus clavatus, Aspergillus nidulans, Aspergillus terreus, Aspergillus ustus, Aspergillus versicolor, or Aspergillus niger. Such ASP antigens include, but are not limited to, antigenic polysaccharides such as any galactofuranose and/or galactofuranose-containing molecules. Galactofuranose is a 5-membered-ring form of galactose present at the surface of many pathogenic pro-and eukaryotes and its presence is detected in the biological fluid of patients with invasive aspergillosis. In certain preferred embodiments, the antigens disclosed herein may comprise the galactofuranose (GalF) side chain of the Aspergillus cell wall polysaccharide, galactomannan (GM), or fragments thereof. Preferably the antigen comprises (or is) a polysaccharide such as GalF2 (consisting of two beta 1,5- or 1,6-linked galactofuranose subunits), GalF3 (consisting of three beta 1,5- or 1,6-linked galactofuranose subunits), or GalF4 (consisting of four beta 1,5- or beta 1,6-linked galactofuranose subunits), more preferably GalF4. Most preferably, the CAR-targeted antigen comprises (or is) GalF4, wherein the subunits are beta 1,5-linked In some embodiments, the antigen targeted by the CAR is GalF4 and is associated with an Aspergillus-associated disease. In preferred embodiments, ASP is targeted by an immune effector cell (i.e., a T cell, Natural Killer (NK) cell, or a pluripotent stem cell that can differentiate into an immune effector cell such as a cytotoxic T cell) that is engineered to express a chimeric antigen receptor (CAR) polypeptide that selectively binds ASP.

A major advance for T cell therapy was the development of chimeric antigen receptors (CARs). First generation CARs were developed as an artificial receptor that, when expressed by T cells, could retarget them to a predetermined disease-associated antigen (e.g., tumor-, viral- or fungal-associated antigens). Such CARs typically comprise a single chain variable fragment (scFv) derived from a target-specific antibody, fused to signaling domains from a T cell receptor (TCR), such as CD3ζ. Upon binding antigen, CARs trigger phosphorylation of immunoreceptor tyrosine-based activation motifs (ITAMS) and initiate the signal cascade required for cytolysis, cytokine secretion and proliferation, bypassing the endogenous antigen-processing pathway and MHC restriction. Second generation CAR designs include further signaling domains to enhance activation and co-stimulation, such as CD28 and/or 4-IBB. Compared to their earlier counterparts, second generation CARs were observed to induce more IL-2 secretion, increase T cell proliferation and persistence, mediate greater tumor rejection, and extend T cell survival. The third generation CARs are made by combining multiple signaling domains, such as CD3ζ-CD28-OC40 or CD3ζ -CD28-4IBB, to augment potency with stronger cytokine production and killing ability.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are anti-GalF antibody clones EB-A1, EB-A2, EB-A3, EB-A4, EB-A5, EB-A6, or EB-A7 (Stynen et al., Infect. Immun. 60(6):2237-2245 (1992), incorporated by reference herein in its entirety). In addition to intact immunoglobulin molecules, also included in the term “antibodies” are chimeras, fragments, or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody’s specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multi-specific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc.), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody’s antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3’ position of one nucleotide to the 5’ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment” or “scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically, specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

The term “ASP antigen” is intended to encompass antigenic epitopes expressed by Aspergillus and which are targetable by CARs, and include fragments, variants (e.g., allelic variants), and derivatives of the native antigen molecule.

### Chimeric Antigen Receptors (CAR)

Disclosed herein are chimeric antigen receptor (CAR) polypeptides that can be expressed in immune effector cells to enhance anti-ASP activity against Aspergillus, such as Aspergillus fumigatus, Aspergillusflavus, Aspergillus clavatus, Aspergillus nidulans, Aspergillus terreus, Aspergillus ustus, Aspergillus versicolor, or Aspergillus niger for the treatment of Aspergillus-associated diseases and disorders in humans. Preferably, such CAR-expressing cells enhance anti-ASP activity against Aspergillusfumigatus and/or Aspergillus niger.

In some aspects, the CARs disclosed herein are made up of three domains: an ectodomain, a transmembrane domain, and an endodomain.

In certain embodiments, the ectodomain of the CAR comprises a ASP-binding region, such as a GalF4-binding region, and is responsible for antigen recognition. The ectodomain also optionally contains a signal peptide (SP) so that the CAR can be glycosylated and anchored in the cell membrane of the immune effector cell.

In some embodiments, the transmembrane domain (TD) connects the ectodomain (i.e., the extracellular domain) to the endodomain (i.e., the intracellular domain) and resides within the cell membrane when expressed by a cell.

In some embodiments, the endodomain transmits an activation signal to the immune effector cell after antigen recognition. In some such embodiments, the endodomain can contain an intracellular signaling domain (ISD) and, optionally, a co-stimulatory signaling region (CSR). A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:
SP—ASP—HG—TM—CSR—SD; or
SP—ASP—HG—TM—SD—CSR;
wherein “SP” represents an optional signal peptide,
wherein “ASP” represents a ASP antigen binding region, which preferably may be a GalF4 antigen binding region,
wherein “HG” represents an optional hinge domain,
wherein “TM” represents a transmembrane domain,
wherein “CSR” represents one or more co-stimulatory signaling regions,
wherein “SD” represents a signaling domain, and
wherein “-” represents a peptide bond or linker.

Additional CAR constructs are described, for example, in Fresnak et al., Nat. Rev. Cancer 16(9):566-81 (2016), which is incorporated by reference in its entirety for the teaching of these CAR models.

In certain embodiments, the CAR can be for example (and without limitation), a TRUCK, Universal CAR, a Self-driving CAR, an Armored CAR, a Self-destruct CAR, a Conditional CAR, a Marked CAR, a TenCAR, a Dual CAR, or a sCAR.

TRUCKs (T cells redirected for universal cytokine killing) co-express a chimeric antigen receptor (CAR) and a pro-inflammatory cytokine. Cytokine expression may be constitutive or induced by T cell activation. Targeted by CAR specificity, localized production of pro-inflammatory cytokines recruits endogenous immune cells to infection sites and may potentiate an antifungal response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds, for example, inflammatory cytokines, thereby enhancing chemo-attraction and targeting.

CAR T cells engineered to be resistant to immunosuppression (Armored CARs) may be genetically modified to no longer be susceptible to immunosuppression and/or immune-evasion s(e.g., gliotoxin, CCR7 deficient dendritic cells, or other immunosuppressive/ immunomodulatory therapy). Exemplary “Knockdown” and “Knockout” techniques for such genetic modification include, but are not limited to, RNA interference (RNAi) (e.g., asRNA, miRNA, shRNA, siRNA, etc.) and CRISPR interference (CRISPRi) (e.g., CRISPR-Cas9). In certain embodiments, CAR T cells are engineered to express a dominant-negative form of a molecule. In some such embodiments, the extracellular ligand-binding domain (i.e., ectodomain) of the molecule is fused to a transmembrane membrane in order to compete for ligand binding. For example, the extracellular ligand-binding domain may be fused to a CD8 transmembrane domain, thus competing for immunosuppressive and/ or immune-evasive ligands from the target cell. In some embodiments, CAR T cells are engineered to express a switch receptor to exploit the immunosuppressive and/ or immune-evasive ligand of the target cell. In such embodiments, the extracellular ligand-binding domain of the immunosuppressive and/ or immuneevasivemolecule is fused to a signaling, stimulatory, and/or co-stimulatory domain. In further embodiments, the CAR T cells may be administered with an aptamer or a monoclonal antibody that blocks immunosuppressive and/ or immune-evasive signalingIn yet further embodiments, the CAR T cells are administered with a vector (e.g., an engineered virus) that expresses an immunosuppression and/ or immuno-evasion-blocking molecule.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus an epitope to which an existing monoclonal antibody agent binds. In the setting of intolerable adverse effects, administration of the monoclonal antibody clears the CAR T cells and alleviates symptoms with no additional off-target effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the costimulatory domain(s). Dual CAR T cell activation requires co-expression of both targets on the pathogen (e.g. Aspergillus conidia and/ or hyphae).

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

In some embodiments, the antigen recognition domain or antigen binding domain of the disclosed CAR is an scFv that recognizes and binds to the antigen of interest. In further embodiments, the antigen recognition domain is from native T-cell receptor (TCR) alpha and beta single chains as are described herein. Preferably, such antigen recognition domains have simple ectodomains (e.g. aCD4 ectodomain to recognize HIV infected cells). Alternatively, such antigen recognition domains comprise exotic recognition components such as a linked cytokine (which leads to recognition of cells bearing the cytokine receptor). Generally, with respect to the compositions and methods disclosed herein, almost anything that binds a given target with high affinity can be used as an antigen recognition region.

The intracellular endodomain transmits a signal to the immune effector cell expressing the CAR after antigen recognition, activating at least one of the normal effector functions of said immune effector cell. In certain embodiments, the effector function of a T cell, for example, may be cytolytic activity or helper activity, including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcyRIy, FcyRIIIy, FcεRIβ (FCERIB), and FcεRIγ (FCERIG).

In particular embodiments, the intracellular signaling domain is derived from CD3 zeta (CD3ζ) (TCR zeta, GenBank acc. no. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene. The intracellular tails of the CD3 molecules contain a single ITAM, which is essential for the signaling capacity of the TCR. The intracellular tail of the ξ chain (CD3ζ) contains 3 ITAMs. In some embodiments, the CD3ζ chain is a mutant CD3ζ chain. For example, the mutant CD3ζ chain comprises a mutation, such as a point mutation, in at least one ITAM so as to render said ITAM non-functional. In some such embodiments, either the membrane-proximal ITAM (ITAM1), the membrane-distal ITAM (C-terminal third ITAM, ITAM3), or both are non-functional. In further embodiments, either two membrane-proximal ITAMs (ITAM1 and ITAM2) or two membrane-distal ITAMs (ITAM2 and ITAM3) are non-functional. In yet further embodiments, only ITAM2 is non-functional. In some embodiments, the mutant CD3ζ chain comprises a deletion (e.g., truncation) mutation such that at least one ITAM is missing. In some such embodiments, the CD3ζ chain is missing the membrane-proximal ITAM (ITAM1), the membrane-distal ITAM (ITAM3), or both. In other embodiments, the CD3ζ chain is missing either two membrane-proximal ITAMs (ITAM1 and ITAM2) or two membrane-distal ITAMs (ITAM2 and ITAM3). In further embodiments, the CD3ζ chain is missing ITAM2. Methods to produce mutant CD3ζ is known to those skilled in the art (Bridgeman et al., Clin. Exp. Immunol. 175(2):258-67 (2014)). Removing at least one ITAM from the introduced CAR may reduce CD3ζmediated apoptosis. Alternatively, removing at least one ITAM from the introduced CAR can reduce its size without loss of function. CARs comprising such altered CD3ζ domains are contemplated by the present invention.

Also contemplated are CARs comprising an altered CD28 domain that imparts unique functional properties to the CAR. In this regard, the native CD28 domain comprises three intracellular subdomains consisting of the amino acid sequences YMNM, PRRP, and PYAP that regulate signaling pathways post stimulation (see, e.g., WO 2019/010383 incorporated herein by reference for this teaching). The CAR constructs described herein may comprise a modified CD28 domain wherein one or more of the YMNM, PRRP, and/or PYAP subdomains are mutated or deleted, so as to amplify, attenuate, or inactivate said subdomain(s), thereby modulating CAR-T function. In a preferred embodiment, the altered CD28 domain employed is Mut06 as described in WO 2019/010383.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai et al., Leukemia 18:676-84 (2004) and Maher et al., Nat. Biotechnol. 20:70-5 (2002)).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol. 4(2): 89-99 (2004)). The hinge sequence may be positioned between the antigen recognition moiety (e.g., an anti-ASP scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e., comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD 11d, ITGAE, CD 103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55) , PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively, the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the CAR contains one signaling domain. In other embodiments, the CAR contains one or more signaling domain (co-stimulatory signaling domain). The one or more signaling domain may be a polypeptide selected from: CD8, CD3ζ, CD3δ, CD3γ, CD3ε, FcγRI-γ, FcyRIII-y, FcεRIβ, FcεRIy, DAP10, DAP12, CD32, CD79a, CD79b, CD28, CD3C, CD4, b2c, CD137 (41BB), ICOS, CD27, CD288, CD80, NKp30, OX40, and mutants thereof.

Tables 1, 2, and 3 below provide some example combinations of ASP antigen-binding region, co-stimulatory signaling regions, and intracellular signaling domain that can occur in the disclosed CARs.

Table 1

First Generation CARs

| | |
|---|---|
| ScFv | Signal Domain |
| ASP | CD8 |
| ASP | CD3ζ |
| ASP | CD38 |
| ASP | CD3γ |
| ASP | CD3ε |
| ASP | FcγRI-γ |
| ASP | FcγRIII-γ |
| ASP | FcεRIβ |
| ASP | FcεRIγ |
| ASP | DAP10 |
| ASP | DAP12 |
| ASP | CD32 |
| ASP | CD79a |

Table 2

Second Generation CARs

| | | | | | |
|---|---|---|---|---|---|
| ScFv | Co-stimulatory Signal | Signal Domain | ScFv | Co-stimulatory Signal | Signal Domain |
| ASP | CD28 | CD8 | ASP | CD80 | FcεRIβ |
| ASP | CD28 | CD3ζ | ASP | CD80 | FcεRIγ |
| ASP | CD28 | CD3δ | ASP | CD80 | DAP10 |
| ASP | CD28 | CD3γ | ASP | CD80 | DAP12 |
| ASP | CD28 | CD3ε | ASP | CD80 | CD32 |
| ASP | CD28 | FcγRI-γ | ASP | CD80 | CD79a |
| ASP | CD28 | FcγRIII-γ | ASP | CD80 | CD79b |
| ASP | CD28 | FcεRIβ | ASP | CD86 | CD8 |
| ASP | CD28 | FcεRIγ | ASP | CD86 | CD3ζ |
| ASP | CD28 | DAP10 | ASP | CD86 | CD3δ |
| ASP | CD28 | DAP12 | ASP | CD86 | CD3γ |
| ASP | CD28 | CD32 | ASP | CD86 | CD3ε |
| ASP | CD28 | CD79a | ASP | CD86 | FcγRI-γ |
| ASP | CD28 | CD79b | ASP | CD86 | FcγRIII-γ |
| ASP | CD8 | CD8 | ASP | CD86 | FcεRIβ |
| ASP | CD8 | CD3ζ | ASP | CD86 | FcεRIγ |
| ASP | CD8 | CD3δ | ASP | CD86 | DAP10 |
| ASP | CD8 | CD3γ | ASP | CD86 | DAP12 |
| ASP | CD8 | CD3ε | ASP | CD86 | CD32 |
| ASP | CD8 | FcγRI-γ | ASP | CD86 | CD79a |
| ASP | CD8 | FcγRIII-γ | ASP | CD86 | CD79b |
| ASP | CD8 | FcεRIβ | ASP | OX40 | CD8 |
| ASP | CD8 | FcεRIγ | ASP | OX40 | CD3ζ |
| ASP | CD8 | DAP10 | ASP | OX40 | CD3δ |
| ASP | CD8 | DAP12 | ASP | OX40 | CD3γ |
| ASP | CD8 | CD32 | ASP | OX40 | CD3ε |
| ASP | CD8 | CD79a | ASP | OX40 | FcγRI-γ |
| ASP | CD8 | CD79b | ASP | OX40 | FcγRIII-γ |
| ASP | CD4 | CD8 | ASP | OX40 | FcεRIβ |
| ASP | CD4 | CD3ζ | ASP | OX40 | FcεRIγ |
| ASP | CD4 | CD3δ | ASP | OX40 | DAP10 |
| ASP | CD4 | CD3γ | ASP | OX40 | DAP12 |
| ASP | CD4 | CD3ε | ASP | OX40 | CD32 |
| ASP | CD4 | FcγRI-γ | ASP | OX40 | CD79a |
| ASP | CD4 | FcγRIII-γ | ASP | OX40 | CD79b |
| ASP | CD4 | FcεRIβ | ASP | DAP10 | CD8 |
| ASP | CD4 | FcεRIγ | ASP | DAP10 | CD3ξ |
| ASP | CD4 | DAP10 | ASP | DAP10 | CD3δ |
| ASP | CD4 | DAP12 | ASP | DAP10 | CD3γ |
| ASP | CD4 | CD32 | ASP | DAP10 | CD3ε |
| ASP | CD4 | CD79a | ASP | DAP10 | FcγRI-γ |
| ASP | CD4 | CD79b | ASP | DAP10 | FcγRIII-γ |
| ASP | b2c | CD8 | ASP | DAP10 | FcεRIβ |
| ASP | b2c | CD3ζ | ASP | DAP10 | FcεRIγ |
| ASP | b2c | CD3δ | ASP | DAP10 | DAP10 |
| ASP | b2c | CD3γ | ASP | DAP10 | DAP12 |
| ASP | b2c | CD3ε | ASP | DAP10 | CD32 |
| ASP | b2c | FcγRI-γ | ASP | DAP10 | CD79a |
| ASP | b2c | FcγRIII-γ | ASP | DAP10 | CD79b |
| ASP | b2c | FcεRIβ | ASP | DAP12 | CD8 |
| ASP | b2c | FcεRIγ | ASP | DAP12 | CD3ζ |
| ASP | b2c | DAP10 | ASP | DAP12 | CD3δ |
| ASP | b2c | DAP12 | ASP | DAP12 | CD3γ |
| ASP | b2c | CD32 | ASP | DAP12 | CD3ε |
| ASP | b2c | CD79a | ASP | DAP12 | FcγRI-γ |
| ASP | b2c | CD79b | ASP | DAP12 | FcγRIII-γ |
| ASP | CD137/41BB | CD8 | ASP | DAP12 | FcεRIβ |
| ASP | CD137/41BB | CD3ζ | ASP | DAP12 | FcεRIγ |
| ASP | CD137/41BB | CD3δ | ASP | DAP12 | DAP10 |
| ASP | CD137/41BB | CD3γ | ASP | DAP12 | DAP12 |
| ASP | CD137/41BB | CD3ε | ASP | DAP12 | CD32 |
| ASP | CD137/41BB | FcγRI-γ | ASP | DAP12 | CD79a |
| ASP | CD137/41BB | FcγRIII-γ | ASP | DAP12 | CD79b |
| ASP | CD137/41BB | FcεRIβ | ASP | MyD88 | CD8 |
| ASP | CD137/41BB | FcεRIγ | ASP | MyD88 | CD3ζ |
| ASP | CD137/41BB | DAP10 | ASP | MyD88 | CD3δ |
| ASP | CD137/41BB | DAP12 | ASP | MyD88 | CD3γ |
| ASP | CD137/41BB | CD32 | ASP | MyD88 | CD3ε |
| ASP | CD137/41BB | CD79a | ASP | MyD88 | FcγRI-γ |
| ASP | CD137/41BB | CD79b | ASP | MyD88 | FcγRIII-γ |
| ASP | ICOS | CD8 | ASP | MyD88 | FcεRIβ |
| ASP | ICOS | CD3ζ | ASP | MyD88 | FcεRIγ |
| ASP | ICOS | CD3δ | ASP | MyD88 | DAP10 |
| ASP | ICOS | CD3γ | ASP | MyD88 | DAP12 |
| ASP | ICOS | CD3ε | ASP | MyD88 | CD32 |
| ASP | ICOS | FcγRI-γ | ASP | MyD88 | CD79a |
| ASP | ICOS | FcγRIII-γ | ASP | MyD88 | CD79b |
| ASP | ICOS | FcεRIβ | ASP | CD7 | CD8 |
| ASP | ICOS | FcεRIγ | ASP | CD7 | CD3ζ |
| ASP | ICOS | DAP10 | ASP | CD7 | CD3δ |
| ASP | ICOS | DAP12 | ASP | CD7 | CD3γ |
| ASP | ICOS | CD32 | ASP | CD7 | CD3ε |
| ASP | ICOS | CD79a | ASP | CD7 | FcγRI-γ |
| ASP | ICOS | CD79b | ASP | CD7 | FcγRIII-γ |
| ASP | CD27 | CD8 | ASP | CD7 | FcεRIβ |
| ASP | CD27 | CD3ζ | ASP | CD7 | FcεRIγ |
| ASP | CD27 | CD3δ | ASP | CD7 | DAP10 |
| ASP | CD27 | CD3γ | ASP | CD7 | DAP12 |
| ASP | CD27 | CD3ε | ASP | CD7 | CD32 |
| ASP | CD27 | FcγRI-γ | ASP | CD7 | CD79a |
| ASP | CD27 | FcγRIII-γ | ASP | CD7 | CD79b |
| ASP | CD27 | FcεRIβ | ASP | BTNL3 | CD8 |
| ASP | CD27 | FcεRIγ | ASP | BTNL3 | CD3ζ |
| ASP | CD27 | DAP10 | ASP | BTNL3 | CD3δ |
| ASP | CD27 | DAP12 | ASP | BTNL3 | CD3γ |
| ASP | CD27 | CD32 | ASP | BTNL3 | CD3ε |
| ASP | CD27 | CD79a | ASP | BTNL3 | FcγRI-γ |
| ASP | CD27 | CD79b | ASP | BTNL3 | FcγRIII-γ |
| ASP | CD28δ | CD8 | ASP | BTNL3 | FcεRIβ |
| ASP | CD28δ | CD3ζ | ASP | BTNL3 | FcεRIγ |
| ASP | CD28δ | CD3δ | ASP | BTNL3 | DAP10 |
| ASP | CD28δ | CD3γ | ASP | BTNL3 | DAP12 |
| ASP | CD28δ | CD3ε | ASP | BTNL3 | CD32 |
| ASP | CD28δ | FcγRI-γ | ASP | BTNL3 | CD79a |
| ASP | CD28δ | FcγRIII-γ | ASP | BTNL3 | CD79b |
| ASP | CD28δ | FcεRIβ | ASP | NKG2D | CD8 |
| ASP | CD28δ | FcεRIγ | ASP | NKG2D | CD3ζ |
| ASP | CD28δ | DAP10 | ASP | NKG2D | CD3δ |
| ASP | CD28δ | DAP12 | ASP | NKG2D | CD3γ |
| ASP | CD28δ | CD32 | ASP | NKG2D | CD3ε |
| ASP | CD28δ | CD79a | ASP | NKG2D | FcγRI-γ |
| ASP | CD28δ | CD79b | ASP | NKG2D | FcγRIII-γ |
| ASP | CD80 | CD8 | ASP | NKG2D | FcεRIβ |
| ASP | CD80 | CD3ζ | ASP | NKG2D | FcεRIγ |
| ASP | CD80 | CD3δ | ASP | NKG2D | DAP10 |
| ASP | CD80 | CD3γ | ASP | NKG2D | DAP12 |
| ASP | CD80 | CD3ε | ASP | NKG2D | CD32 |
| ASP | CD80 | FcγRI-γ | ASP | NKG2D | CD79a |
| ASP | CD80 | FcγRIII-γ | ASP | NKG2D | CD79b |

Table 3

Third Generation CARs

| ScFv | Co-stimulatory Signal | Co-stimulatory Signal | Signal Domain |
|---|---|---|---|
| ASP | CD28 | CD28 | CD8 |
| ASP | CD28 | CD28 | CD3ζ |
| ASP | CD28 | CD28 | CD3δ |
| ASP | CD28 | CD28 | CD3γ |
| ASP | CD28 | CD28 | CD3ε |
| ASP | CD28 | CD28 | FcγRI-γ |
| ASP | CD28 | CD28 | FcγRIII-γ |
| ASP | CD28 | CD28 | FcεRIβ |
| ASP | CD28 | CD28 | FcεRIγ |
| ASP | CD28 | CD28 | DAP10 |
| ASP | CD28 | CD28 | DAP12 |
| ASP | CD28 | CD28 | CD32 |
| ASP | CD28 | CD28 | CD79a |
| ASP | CD28 | CD28 | CD79b |
| ASP | CD28 | CD8 | CD8 |
| ASP | CD28 | CD8 | CD3ζ |
| ASP | CD28 | CD8 | CD3δ |
| ASP | CD28 | CD8 | CD3γ |
| ASP | CD28 | CD8 | CD3ε |
| ASP | CD28 | CD8 | FcγRI-γ |
| ASP | CD28 | CD8 | FcγRIII-γ |
| ASP | CD28 | CD8 | FcεRIβ |
| ASP | CD28 | CD8 | FcεRIγ |
| ASP | CD28 | CD8 | DAP10 |
| ASP | CD28 | CD8 | DAP12 |
| ASP | CD28 | CD8 | CD32 |
| ASP | CD28 | CD8 | CD79a |
| ASP | CD28 | CD8 | CD79b |
| ASP | CD28 | CD4 | CD8 |
| ASP | CD28 | CD4 | CD3ζ |
| ASP | CD28 | CD4 | CD38 |
| ASP | CD28 | CD4 | CD3γ |
| ASP | CD28 | CD4 | CD3ε |
| ASP | CD28 | CD4 | FcγRI-γ |
| ASP | CD28 | CD4 | FcγRIII-γ |
| ASP | CD28 | CD4 | FcεRIβ |
| ASP | CD28 | CD4 | FcεRIγ |
| ASP | CD28 | CD4 | DAP10 |
| ASP | CD28 | CD4 | DAP12 |
| ASP | CD28 | CD4 | CD32 |
| ASP | CD28 | CD4 | CD79a |
| ASP | CD28 | CD4 | CD79b |
| ASP | CD28 | b2c | CD8 |
| ASP | CD28 | b2c | CD3ζ |
| ASP | CD28 | b2c | CD3δ |
| ASP | CD28 | b2c | CD3γ |
| ASP | CD28 | b2c | CD3ε |
| ASP | CD28 | b2c | FcγRI-γ |
| ASP | CD28 | b2c | FcγRIII-γ |
| ASP | CD28 | b2c | FcεRIβ |
| ASP | CD28 | b2c | FcεRIγ |
| ASP | CD28 | b2c | DAP10 |
| ASP | CD28 | b2c | DAP12 |
| ASP | CD28 | b2c | CD32 |
| ASP | CD28 | b2c | CD79a |
| ASP | CD28 | b2c | CD79b |
| ASP | CD28 | CD137/41BB | CD8 |
| ASP | CD28 | CD137/41BB | CD3ζ |
| ASP | CD28 | CD137/41BB | CD3δ |
| ASP | CD28 | CD137/41BB | CD3γ |
| ASP | CD28 | CD137/41BB | CD3ε |
| ASP | CD28 | CD137/41BB | FcγRI-γ |
| ASP | CD28 | CD137/41BB | FcγRIII-γ |
| ASP | CD28 | CD137/41BB | FcεRIβ |
| ASP | CD28 | CD137/41BB | FcεRIγ |
| ASP | CD28 | CD137/41BB | DAP10 |
| ASP | CD28 | CD137/41BB | DAP12 |
| ASP | CD28 | CD137/41BB | CD32 |
| ASP | CD28 | CD137/41BB | CD79a |
| ASP | CD28 | CD137/41BB | CD79b |
| ASP | CD28 | ICOS | CD8 |
| ASP | CD28 | ICOS | CD3ζ |
| ASP | CD28 | ICOS | CD3δ |
| ASP | CD28 | ICOS | CD3γ |
| ASP | CD28 | ICOS | CD3ε |
| ASP | CD28 | ICOS | FcγRI-γ |
| ASP | CD28 | ICOS | FcγRIII-γ |
| ASP | CD28 | ICOS | FcεRIβ |
| ASP | CD28 | ICOS | FcεRIγ |
| ASP | CD28 | ICOS | DAP10 |
| ASP | CD28 | ICOS | DAP12 |
| ASP | CD28 | ICOS | CD32 |
| ASP | CD28 | ICOS | CD79a |
| ASP | CD28 | ICOS | CD79b |
| ASP | CD28 | CD27 | CD8 |
| ASP | CD28 | CD27 | CD3ζ |
| ASP | CD28 | CD27 | CD3δ |
| ASP | CD28 | CD27 | CD3γ |
| ASP | CD28 | CD27 | CD3ε |
| ASP | CD28 | CD27 | FcγRI-γ |
| ASP | CD28 | CD27 | FcγRIII-γ |
| ASP | CD28 | CD27 | FcεRIβ |
| ASP | CD28 | CD27 | FcεRIγ |
| ASP | CD28 | CD27 | DAP10 |
| ASP | CD28 | CD27 | DAP12 |
| ASP | CD28 | CD27 | CD32 |
| ASP | CD28 | CD27 | CD79a |
| ASP | CD28 | CD27 | CD79b |
| ASP | CD28 | CD28δ | CD8 |
| ASP | CD28 | CD28δ | CD3ζ |
| ASP | CD28 | CD28δ | CD3δ |
| ASP | CD28 | CD28δ | CD3γ |
| ASP | CD28 | CD28δ | CD3ε |
| ASP | CD28 | CD28δ | FcγRI-γ |
| ASP | CD28 | CD28δ | FcγRIII-γ |
| ASP | CD28 | CD28δ | FcεRIβ |
| ASP | CD28 | CD28δ | FcεRIγ |
| ASP | CD28 | CD28δ | DAP10 |
| ASP | CD28 | CD28δ | DAP12 |
| ASP | CD28 | CD28δ | CD32 |
| ASP | CD28 | CD28δ | CD79a |
| ASP | CD28 | CD28δ | CD79b |
| ASP | CD28 | CD80 | CD8 |
| ASP | CD28 | CD80 | CD3ζ |
| ASP | CD28 | CD80 | CD3δ |
| ASP | CD28 | CD80 | CD3γ |
| ASP | CD28 | CD80 | CD3ε |
| ASP | CD28 | CD80 | FcγRI-γ |
| ASP | CD28 | CD80 | FcγRIII-γ |
| ASP | CD28 | CD80 | FcεRIβ |
| ASP | CD28 | CD80 | FcεRIγ |
| ASP | CD28 | CD80 | DAP10 |
| ASP | CD28 | CD80 | DAP12 |
| ASP | CD28 | CD80 | CD32 |
| ASP | CD28 | CD80 | CD79a |
| ASP | CD28 | CD80 | CD79b |
| ASP | CD28 | CD86 | CD8 |
| ASP | CD28 | CD86 | CD3ζ |
| ASP | CD28 | CD86 | CD3δ |
| ASP | CD28 | CD86 | CD3γ |
| ASP | CD28 | CD86 | CD3ε |
| ASP | CD28 | CD86 | FcγRI-γ |
| ASP | CD28 | CD86 | FcγRIII-γ |
| ASP | CD28 | CD86 | FcεRIβ |
| ASP | CD28 | CD86 | FcεRIγ |
| ASP | CD28 | CD86 | DAP10 |
| ASP | CD28 | CD86 | DAP12 |
| ASP | CD28 | CD86 | CD32 |
| ASP | CD28 | CD86 | CD79a |
| ASP | CD28 | CD86 | CD79b |
| ASP | CD28 | OX40 | CD8 |
| ASP | CD28 | OX40 | CD3ζ |
| ASP | CD28 | OX40 | CD3δ |
| ASP | CD28 | OX40 | CD3γ |
| ASP | CD28 | OX40 | CD3ε |
| ASP | CD28 | OX40 | FcγRI-γ |
| ASP | CD28 | OX40 | FcγRIII-γ |
| ASP | CD28 | OX40 | FcεRIβ |
| ASP | CD28 | OX40 | FcεRIγ |
| ASP | CD28 | OX40 | DAP10 |
| ASP | CD28 | OX40 | DAP12 |
| ASP | CD28 | OX40 | CD32 |
| ASP | CD28 | OX40 | CD79a |
| ASP | CD28 | OX40 | CD79b |
| ASP | CD28 | DAP10 | CD8 |
| ASP | CD28 | DAP10 | CD3ζ |
| ASP | CD28 | DAP10 | CD3δ |
| ASP | CD28 | DAP10 | CD3γ |
| ASP | CD28 | DAP10 | CD3ε |
| ASP | CD28 | DAP10 | FcγRI-γ |
| ASP | CD28 | DAP10 | FcγRIII-γ |
| ASP | CD28 | DAP10 | FcεRIβ |
| ASP | CD28 | DAP10 | FcεRIγ |
| ASP | CD28 | DAP10 | DAP10 |
| ASP | CD28 | DAP10 | DAP12 |
| ASP | CD28 | DAP10 | CD32 |
| ASP | CD28 | DAP10 | CD79a |
| ASP | CD28 | DAP10 | CD79b |
| ASP | CD28 | DAP12 | CD8 |
| ASP | CD28 | DAP12 | CD3ζ |
| ASP | CD28 | DAP12 | CD3δ |
| ASP | CD28 | DAP12 | CD3γ |
| ASP | CD28 | DAP12 | CD3ε |
| ASP | CD28 | DAP12 | FcγRI-γ |
| ASP | CD28 | DAP12 | FcγRIII-γ |
| ASP | CD28 | DAP12 | FcεRIβ |
| ASP | CD28 | DAP12 | FcεRIγ |
| ASP | CD28 | DAP12 | DAP10 |
| ASP | CD28 | DAP12 | DAP12 |
| ASP | CD28 | DAP12 | CD32 |
| ASP | CD28 | DAP12 | CD79a |
| ASP | CD28 | DAP12 | CD79b |
| ASP | CD28 | MyD88 | CD8 |
| ASP | CD28 | MyD88 | CD3ζ |
| ASP | CD28 | MyD88 | CD3δ |
| ASP | CD28 | MyD88 | CD3γ |
| ASP | CD28 | MyD88 | CD3ε |
| ASP | CD28 | MyD88 | FcγRI-γ |
| ASP | CD28 | MyD88 | FcγRIII-γ |
| ASP | CD28 | MyD88 | FcεRIβ |
| ASP | CD28 | MyD88 | FcεRIγ |
| ASP | CD28 | MyD88 | DAP10 |
| ASP | CD28 | MyD88 | DAP12 |
| ASP | CD28 | MyD88 | CD32 |
| ASP | CD28 | MyD88 | CD79a |
| ASP | CD28 | MyD88 | CD79b |
| ASP | CD28 | CD7 | CD8 |
| ASP | CD28 | CD7 | CD3ζ |
| ASP | CD28 | CD7 | CD3δ |
| ASP | CD28 | CD7 | CD3γ |
| ASP | CD28 | CD7 | CD3ε |
| ASP | CD28 | CD7 | FcγRI-γ |
| ASP | CD28 | CD7 | FcγRIII-γ |
| ASP | CD28 | CD7 | FcεRIβ |
| ASP | CD28 | CD7 | FcεRIγ |
| ASP | CD28 | CD7 | DAP10 |
| ASP | CD28 | CD7 | DAP12 |
| ASP | CD28 | CD7 | CD32 |
| ASP | CD28 | CD7 | CD79a |
| ASP | CD28 | CD7 | CD79b |
| ASP | CD28 | BTNL3 | CD8 |
| ASP | CD28 | BTNL3 | CD3ζ |
| ASP | CD28 | BTNL3 | CD3δ |
| ASP | CD28 | BTNL3 | CD3γ |
| ASP | CD28 | BTNL3 | CD3ε |
| ASP | CD28 | BTNL3 | FcγRI-γ |
| ASP | CD28 | BTNL3 | FcγRIII-γ |
| ASP | CD28 | BTNL3 | FcεRIβ |
| ASP | CD28 | BTNL3 | FcεRIγ |
| ASP | CD28 | BTNL3 | DAP10 |
| ASP | CD28 | BTNL3 | DAP12 |
| ASP | CD28 | BTNL3 | CD32 |
| ASP | CD28 | BTNL3 | CD79a |
| ASP | CD28 | BTNL3 | CD79b |
| ASP | CD28 | NKG2D | CD8 |
| ASP | CD28 | NKG2D | CD3ζ |
| ASP | CD28 | NKG2D | CD3δ |
| ASP | CD28 | NKG2D | CD3γ |
| ASP | CD28 | NKG2D | CD3ε |
| ASP | CD28 | NKG2D | FcγRI-γ |
| ASP | CD28 | NKG2D | FcγRIII-γ |
| ASP | CD28 | NKG2D | FcεRIβ |
| ASP | CD28 | NKG2D | FcεRIγ |
| ASP | CD28 | NKG2D | DAP10 |
| ASP | CD28 | NKG2D | DAP12 |
| ASP | CD28 | NKG2D | CD32 |
| ASP | CD28 | NKG2D | CD79a |
| ASP | CD28 | NKG2D | CD79b |
| ASP | CD8 | CD28 | CD8 |
| ASP | CD8 | CD28 | CD3ζ |
| ASP | CD8 | CD28 | CD3δ |
| ASP | CD8 | CD28 | CD3γ |
| ASP | CD8 | CD28 | CD3ε |
| ASP | CD8 | CD28 | FcγRI-γ |
| ASP | CD8 | CD28 | FcγRIII-γ |
| ASP | CD8 | CD28 | FcεRIβ |
| ASP | CD8 | CD28 | FcεRIγ |
| ASP | CD8 | CD28 | DAP10 |
| ASP | CD8 | CD28 | DAP12 |
| ASP | CD8 | CD28 | CD32 |
| ASP | CD8 | CD28 | CD79a |
| ASP | CD8 | CD28 | CD79b |
| ASP | CD8 | CD8 | CD8 |
| ASP | CD8 | CD8 | CD3ζ |
| ASP | CD8 | CD8 | CD3δ |
| ASP | CD8 | CD8 | CD3γ |
| ASP | CD8 | CD8 | CD3ε |
| ASP | CD8 | CD8 | FcγRI-γ |
| ASP | CD8 | CD8 | FcγRIII-γ |
| ASP | CD8 | CD8 | FcεRIβ |
| ASP | CD8 | CD8 | FcεRIγ |
| ASP | CD8 | CD8 | DAP10 |
| ASP | CD8 | CD8 | DAP12 |
| ASP | CD8 | CD8 | CD32 |
| ASP | CD8 | CD8 | CD79a |
| ASP | CD8 | CD8 | CD79b |
| ASP | CD8 | CD4 | CD8 |
| ASP | CD8 | CD4 | CD3ζ |
| ASP | CD8 | CD4 | CD3δ |
| ASP | CD8 | CD4 | CD3γ |
| ASP | CD8 | CD4 | CD3ε |
| ASP | CD8 | CD4 | FcγRI-γ |
| ASP | CD8 | CD4 | FcγRIII-γ |
| ASP | CD8 | CD4 | FcεRIβ |
| ASP | CD8 | CD4 | FcεRIγ |
| ASP | CD8 | CD4 | DAP10 |
| ASP | CD8 | CD4 | DAP12 |
| ASP | CD8 | CD4 | CD32 |
| ASP | CD8 | CD4 | CD79a |
| ASP | CD8 | CD4 | CD79b |
| ASP | CD8 | b2c | CD8 |
| ASP | CD8 | b2c | CD3ζ |
| ASP | CD8 | b2c | CD3δ |
| ASP | CD8 | b2c | CD3γ |
| ASP | CD8 | b2c | CD3ε |
| ASP | CD8 | b2c | FcγRI-γ |
| ASP | CD8 | b2c | FcγRIII-γ |
| ASP | CD8 | b2c | FcεRIβ |
| ASP | CD8 | b2c | FcεRIγ |
| ASP | CD8 | b2c | DAP10 |
| ASP | CD8 | b2c | DAP12 |
| ASP | CD8 | b2c | CD32 |
| ASP | CD8 | b2c | CD79a |
| ASP | CD8 | b2c | CD79b |
| ASP | CD8 | CD137/41BB | CD8 |
| ASP | CD8 | CD137/41BB | CD3ζ |
| ASP | CD8 | CD137/41BB | CD3δ |
| ASP | CD8 | CD137/41BB | CD3γ |
| ASP | CD8 | CD137/41BB | CD3ε |
| ASP | CD8 | CD137/41BB | FcγRI-γ |
| ASP | CD8 | CD137/41BB | FcγRIII-γ |
| ASP | CD8 | CD137/41BB | FcεRIβ |
| ASP | CD8 | CD137/41BB | FcεRIγ |
| ASP | CD8 | CD137/41BB | DAP10 |
| ASP | CD8 | CD137/41BB | DAP12 |
| ASP | CD8 | CD137/41BB | CD32 |
| ASP | CD8 | CD137/41BB | CD79a |
| ASP | CD8 | CD137/41BB | CD79b |
| ASP | CD8 | ICOS | CD8 |
| ASP | CD8 | ICOS | CD3ζ |
| ASP | CD8 | ICOS | CD3δ |
| ASP | CD8 | ICOS | CD3γ |
| ASP | CD8 | ICOS | CD3ε |
| ASP | CD8 | ICOS | FcyRI-γ |
| ASP | CD8 | ICOS | FcyRIII-γ |
| ASP | CD8 | ICOS | FcεRIβ |
| ASP | CD8 | ICOS | FcεRIγ |
| ASP | CD8 | ICOS | DAP10 |
| ASP | CD8 | ICOS | DAP12 |
| ASP | CD8 | ICOS | CD32 |
| ASP | CD8 | ICOS | CD79a |
| ASP | CD8 | ICOS | CD79b |
| ASP | CD8 | CD27 | CD8 |
| ASP | CD8 | CD27 | CD3ζ |
| ASP | CD8 | CD27 | CD38 |
| ASP | CD8 | CD27 | CD3γ |
| ASP | CD8 | CD27 | CD3ε |
| ASP | CD8 | CD27 | FcyRI-y |
| ASP | CD8 | CD27 | FcyRIII-γ |
| ASP | CD8 | CD27 | FcεRIβ |
| ASP | CD8 | CD27 | FcεRIγ |
| ASP | CD8 | CD27 | DAP10 |
| ASP | CD8 | CD27 | DAP12 |
| ASP | CD8 | CD27 | CD32 |
| ASP | CD8 | CD27 | CD79a |
| ASP | CD8 | CD27 | CD79b |
| ASP | CD8 | CD28δ | CD8 |
| ASP | CD8 | CD28δ | CD3ζ |
| ASP | CD8 | CD28δ | CD3δ |
| ASP | CD8 | CD28δ | CD3γ |
| ASP | CD8 | CD28δ | CD3ε |
| ASP | CD8 | CD28δ | FcyRI-γ |
| ASP | CD8 | CD28δ | FcyRIII-γ |
| ASP | CD8 | CD28δ | FcεRIβ |
| ASP | CD8 | CD28δ | FcεRIγ |
| ASP | CD8 | CD28δ | DAP10 |
| ASP | CD8 | CD28δ | DAP12 |
| ASP | CD8 | CD28δ | CD32 |
| ASP | CD8 | CD28δ | CD79a |
| ASP | CD8 | CD28δ | CD79b |
| ASP | CD8 | CD80 | CD8 |
| ASP | CD8 | CD80 | CD3ζ |
| ASP | CD8 | CD80 | CD38 |
| ASP | CD8 | CD80 | CD3γ |
| ASP | CD8 | CD80 | CD3ε |
| ASP | CD8 | CD80 | FcyRI-γ |
| ASP | CD8 | CD80 | FcyRIII-γ |
| ASP | CD8 | CD80 | FcεRIβ |
| ASP | CD8 | CD80 | FcεRIγ |
| ASP | CD8 | CD80 | DAP10 |
| ASP | CD8 | CD80 | DAP12 |
| ASP | CD8 | CD80 | CD32 |
| ASP | CD8 | CD80 | CD79a |
| ASP | CD8 | CD80 | CD79b |
| ASP | CD8 | CD86 | CD8 |
| ASP | CD8 | CD86 | CD3ζ |
| ASP | CD8 | CD86 | CD38 |
| ASP | CD8 | CD86 | CD3γ |
| ASP | CD8 | CD86 | CD3ε |
| ASP | CD8 | CD86 | FcyRI-γ |
| ASP | CD8 | CD86 | FcyRIII-γ |
| ASP | CD8 | CD86 | FcεRIβ |
| ASP | CD8 | CD86 | FcεRIγ |
| ASP | CD8 | CD86 | DAP10 |
| ASP | CD8 | CD86 | DAP12 |
| ASP | CD8 | CD86 | CD32 |
| ASP | CD8 | CD86 | CD79a |
| ASP | CD8 | CD86 | CD79b |
| ASP | CD8 | OX40 | CD8 |
| ASP | CD8 | OX40 | CD3ζ |
| ASP | CD8 | OX40 | CD3δ |
| ASP | CD8 | OX40 | CD3γ |
| ASP | CD8 | OX40 | CD3ε |
| ASP | CD8 | OX40 | FcyRI-γ |
| ASP | CD8 | OX40 | FcyRIII-γ |
| ASP | CD8 | OX40 | FcεRIβ |
| ASP | CD8 | OX40 | FcεRIγ |
| ASP | CD8 | OX40 | DAP10 |
| ASP | CD8 | OX40 | DAP12 |
| ASP | CD8 | OX40 | CD32 |
| ASP | CD8 | OX40 | CD79a |
| ASP | CD8 | OX40 | CD79b |
| ASP | CD8 | DAP10 | CD8 |
| ASP | CD8 | DAP10 | CD3ζ |
| ASP | CD8 | DAP10 | CD38 |
| ASP | CD8 | DAP10 | CD3γ |
| ASP | CD8 | DAP10 | CD3ε |
| ASP | CD8 | DAP10 | FcyRI-γ |
| ASP | CD8 | DAP10 | FcyRIII-γ |
| ASP | CD8 | DAP10 | FcεRIβ |
| ASP | CD8 | DAP10 | FcεRIγ |
| ASP | CD8 | DAP10 | DAP10 |
| ASP | CD8 | DAP10 | DAP12 |
| ASP | CD8 | DAP10 | CD32 |
| ASP | CD8 | DAP10 | CD79a |
| ASP | CD8 | DAP10 | CD79b |
| ASP | CD8 | DAP12 | CD8 |
| ASP | CD8 | DAP12 | CD3ζ |
| ASP | CD8 | DAP12 | CD38 |
| ASP | CD8 | DAP12 | CD3γ |
| ASP | CD8 | DAP12 | CD3ε |
| ASP | CD8 | DAP12 | FcyRI-γ |
| ASP | CD8 | DAP12 | FcyRIII-γ |
| ASP | CD8 | DAP12 | FcεRIβ |
| ASP | CD8 | DAP12 | FcεRIγ |
| ASP | CD8 | DAP12 | DAP10 |
| ASP | CD8 | DAP12 | DAP12 |
| ASP | CD8 | DAP12 | CD32 |
| ASP | CD8 | DAP12 | CD79a |
| ASP | CD8 | DAP12 | CD79b |
| ASP | CD8 | MyD88 | CD8 |
| ASP | CD8 | MyD88 | CD3ζ |
| ASP | CD8 | MyD88 | CD38 |
| ASP | CD8 | MyD88 | CD3γ |
| ASP | CD8 | MyD88 | CD3ε |
| ASP | CD8 | MyD88 | FcyRI-γ |
| ASP | CD8 | MyD88 | FcyRIII-γ |
| ASP | CD8 | MyD88 | FcεRIβ |
| ASP | CD8 | MyD88 | FcεRIγ |
| ASP | CD8 | MyD88 | DAP10 |
| ASP | CD8 | MyD88 | DAP12 |
| ASP | CD8 | MyD88 | CD32 |
| ASP | CD8 | MyD88 | CD79a |
| ASP | CD8 | MyD88 | CD79b |
| ASP | CD8 | CD7 | CD8 |
| ASP | CD8 | CD7 | CD3ζ |
| ASP | CD8 | CD7 | CD3δ |
| ASP | CD8 | CD7 | CD3γ |
| ASP | CD8 | CD7 | CD3ε |
| ASP | CD8 | CD7 | FcyRI-γ |
| ASP | CD8 | CD7 | FcyRIII-γ |
| ASP | CD8 | CD7 | FcεRIβ |
| ASP | CD8 | CD7 | FcεRIγ |
| ASP | CD8 | CD7 | DAP10 |
| ASP | CD8 | CD7 | DAP12 |
| ASP | CD8 | CD7 | CD32 |
| ASP | CD8 | CD7 | CD79a |
| ASP | CD8 | CD7 | CD79b |
| ASP | CD8 | BTNL3 | CD8 |
| ASP | CD8 | BTNL3 | CD3ζ |
| ASP | CD8 | BTNL3 | CD38 |
| ASP | CD8 | BTNL3 | CD3γ |
| ASP | CD8 | BTNL3 | CD3ε |
| ASP | CD8 | BTNL3 | FcyRI-γ |
| ASP | CD8 | BTNL3 | FcyRIII-γ |
| ASP | CD8 | BTNL3 | FcεRIβ |
| ASP | CD8 | BTNL3 | FcεRIγ |
| ASP | CD8 | BTNL3 | DAP10 |
| ASP | CD8 | BTNL3 | DAP12 |
| ASP | CD8 | BTNL3 | CD32 |
| ASP | CD8 | BTNL3 | CD79a |
| ASP | CD8 | BTNL3 | CD79b |
| ASP | CD8 | NKG2D | CD8 |
| ASP | CD8 | NKG2D | CD3ζ |
| ASP | CD8 | NKG2D | CD38 |
| ASP | CD8 | NKG2D | CD3γ |
| ASP | CD8 | NKG2D | CD3ε |
| ASP | CD8 | NKG2D | FcyRI-γ |
| ASP | CD8 | NKG2D | FcyRIII-γ |
| ASP | CD8 | NKG2D | FcεRIβ |
| ASP | CD8 | NKG2D | FcεRIγ |
| ASP | CD8 | NKG2D | DAP10 |
| ASP | CD8 | NKG2D | DAP12 |
| ASP | CD8 | NKG2D | CD32 |
| ASP | CD8 | NKG2D | CD79a |
| ASP | CD8 | NKG2D | CD79b |
| ASP | CD4 | CD28 | CD8 |
| ASP | CD4 | CD28 | CD3ζ |
| ASP | CD4 | CD28 | CD38 |
| ASP | CD4 | CD28 | CD3γ |
| ASP | CD4 | CD28 | CD3ε |
| ASP | CD4 | CD28 | FcyRI-γ |
| ASP | CD4 | CD28 | FcγRIII-γ |
| ASP | CD4 | CD28 | FcεRIβ |
| ASP | CD4 | CD28 | FcεRIγ |
| ASP | CD4 | CD28 | DAP10 |
| ASP | CD4 | CD28 | DAP12 |
| ASP | CD4 | CD28 | CD32 |
| ASP | CD4 | CD28 | CD79a |
| ASP | CD4 | CD28 | CD79b |
| ASP | CD4 | CD8 | CD8 |
| ASP | CD4 | CD8 | CD3ζ |
| ASP | CD4 | CD8 | CD3δ |
| ASP | CD4 | CD8 | CD3γ |
| ASP | CD4 | CD8 | CD3ε |
| ASP | CD4 | CD8 | FcyRI-γ |
| ASP | CD4 | CD8 | FcyRIII-γ |
| ASP | CD4 | CD8 | FcεRIβ |
| ASP | CD4 | CD8 | FcεRIγ |
| ASP | CD4 | CD8 | DAP10 |
| ASP | CD4 | CD8 | DAP12 |
| ASP | CD4 | CD8 | CD32 |
| ASP | CD4 | CD8 | CD79a |
| ASP | CD4 | CD8 | CD79b |
| ASP | CD4 | CD4 | CD8 |
| ASP | CD4 | CD4 | CD3ζ |
| ASP | CD4 | CD4 | CD3δ |
| ASP | CD4 | CD4 | CD3γ |
| ASP | CD4 | CD4 | CD3ε |
| ASP | CD4 | CD4 | FcyRI-γ |
| ASP | CD4 | CD4 | FcγRIII-γ |
| ASP | CD4 | CD4 | FcεRIβ |
| ASP | CD4 | CD4 | FcεRIγ |
| ASP | CD4 | CD4 | DAP10 |
| ASP | CD4 | CD4 | DAP12 |
| ASP | CD4 | CD4 | CD32 |
| ASP | CD4 | CD4 | CD79a |
| ASP | CD4 | CD4 | CD79b |
| ASP | CD4 | b2c | CD8 |
| ASP | CD4 | b2c | CD3ζ |
| ASP | CD4 | b2c | CD3δ |
| ASP | CD4 | b2c | CD3γ |
| ASP | CD4 | b2c | CD3ε |
| ASP | CD4 | b2c | FcyRI-γ |
| ASP | CD4 | b2c | FcγRIII-γ |
| ASP | CD4 | b2c | FcεRIβ |
| ASP | CD4 | b2c | FcεRIγ |
| ASP | CD4 | b2c | DAP10 |
| ASP | CD4 | b2c | DAP12 |
| ASP | CD4 | b2c | CD32 |
| ASP | CD4 | b2c | CD79a |
| ASP | CD4 | b2c | CD79b |
| ASP | CD4 | CD137/41BB | CD8 |
| ASP | CD4 | CD137/41BB | CD3ζ |
| ASP | CD4 | CD137/41BB | CD38 |
| ASP | CD4 | CD137/41BB | CD3γ |
| ASP | CD4 | CD137/41BB | CD3ε |
| ASP | CD4 | CD137/41BB | FcyRI-γ |
| ASP | CD4 | CD137/41BB | FcγRIII-γ |
| ASP | CD4 | CD137/41BB | FcεRIβ |
| ASP | CD4 | CD137/41BB | FcεRIγ |
| ASP | CD4 | CD137/41BB | DAP10 |
| ASP | CD4 | CD137/41BB | DAP12 |
| ASP | CD4 | CD137/41BB | CD32 |
| ASP | CD4 | CD137/41BB | CD79a |
| ASP | CD4 | CD137/41BB | CD79b |
| ASP | CD4 | ICOS | CD8 |
| ASP | CD4 | ICOS | CD3ζ |
| ASP | CD4 | ICOS | CD3δ |
| ASP | CD4 | ICOS | CD3γ |
| ASP | CD4 | ICOS | CD3ε |
| ASP | CD4 | ICOS | FcyRI-γ |
| ASP | CD4 | ICOS | FcγRIII-γ |
| ASP | CD4 | ICOS | FcεRIβ |
| ASP | CD4 | ICOS | FcεRIγ |
| ASP | CD4 | ICOS | DAP10 |
| ASP | CD4 | ICOS | DAP12 |
| ASP | CD4 | ICOS | CD32 |
| ASP | CD4 | ICOS | CD79a |
| ASP | CD4 | ICOS | CD79b |
| ASP | CD4 | CD27 | CD8 |
| ASP | CD4 | CD27 | CD3ζ |
| ASP | CD4 | CD27 | CD38 |
| ASP | CD4 | CD27 | CD3γ |
| ASP | CD4 | CD27 | CD3ε |
| ASP | CD4 | CD27 | FcyRI-γ |
| ASP | CD4 | CD27 | FcyRIII-γ |
| ASP | CD4 | CD27 | FcεRIβ |
| ASP | CD4 | CD27 | FcεRIγ |
| ASP | CD4 | CD27 | DAP10 |
| ASP | CD4 | CD27 | DAP12 |
| ASP | CD4 | CD27 | CD32 |
| ASP | CD4 | CD27 | CD79a |
| ASP | CD4 | CD27 | CD79b |
| ASP | CD4 | CD28δ | CD8 |
| ASP | CD4 | CD28δ | CD3ζ |
| ASP | CD4 | CD28δ | CD3δ |
| ASP | CD4 | CD28δ | CD3γ |
| ASP | CD4 | CD28δ | CD3ε |
| ASP | CD4 | CD28δ | FcyRI-γ |
| ASP | CD4 | CD28δ | FcyRIII-γ |
| ASP | CD4 | CD28δ | FcεRIβ |
| ASP | CD4 | CD28δ | FcεRIγ |
| ASP | CD4 | CD28δ | DAP10 |
| ASP | CD4 | CD28δ | DAP12 |
| ASP | CD4 | CD28δ | CD32 |
| ASP | CD4 | CD28δ | CD79a |
| ASP | CD4 | CD28δ | CD79b |
| ASP | CD4 | CD80 | CD8 |
| ASP | CD4 | CD80 | CD3ζ |
| ASP | CD4 | CD80 | CD38 |
| ASP | CD4 | CD80 | CD3γ |
| ASP | CD4 | CD80 | CD3ε |
| ASP | CD4 | CD80 | FcyRI-γ |
| ASP | CD4 | CD80 | FcyRIII-γ |
| ASP | CD4 | CD80 | FcεRIβ |
| ASP | CD4 | CD80 | FcεRIγ |
| ASP | CD4 | CD80 | DAP10 |
| ASP | CD4 | CD80 | DAP12 |
| ASP | CD4 | CD80 | CD32 |
| ASP | CD4 | CD80 | CD79a |
| ASP | CD4 | CD80 | CD79b |
| ASP | CD4 | CD86 | CD8 |
| ASP | CD4 | CD86 | CD3ζ |
| ASP | CD4 | CD86 | CD38 |
| ASP | CD4 | CD86 | CD3γ |
| ASP | CD4 | CD86 | CD3ε |
| ASP | CD4 | CD86 | FcyRI-γ |
| ASP | CD4 | CD86 | FcyRIII-γ |
| ASP | CD4 | CD86 | FcεRIβ |
| ASP | CD4 | CD86 | FcεRIγ |
| ASP | CD4 | CD86 | DAP10 |
| ASP | CD4 | CD86 | DAP12 |
| ASP | CD4 | CD86 | CD32 |
| ASP | CD4 | CD86 | CD79a |
| ASP | CD4 | CD86 | CD79b |
| ASP | CD4 | OX40 | CD8 |
| ASP | CD4 | OX40 | CD3ζ |
| ASP | CD4 | OX40 | CD3δ |
| ASP | CD4 | OX40 | CD3γ |
| ASP | CD4 | OX40 | CD3ε |
| ASP | CD4 | OX40 | FcyRI-γ |
| ASP | CD4 | OX40 | FcyRIII-γ |
| ASP | CD4 | OX40 | FcεRIβ |
| ASP | CD4 | OX40 | FcεRIγ |
| ASP | CD4 | OX40 | DAP10 |
| ASP | CD4 | OX40 | DAP12 |
| ASP | CD4 | OX40 | CD32 |
| ASP | CD4 | OX40 | CD79a |
| ASP | CD4 | OX40 | CD79b |
| ASP | CD4 | DAP10 | CD8 |
| ASP | CD4 | DAP10 | CD3ζ |
| ASP | CD4 | DAP10 | CD38 |
| ASP | CD4 | DAP10 | CD3γ |
| ASP | CD4 | DAP10 | CD3ε |
| ASP | CD4 | DAP10 | FcyRI-γ |
| ASP | CD4 | DAP10 | FcyRIII-γ |
| ASP | CD4 | DAP10 | FcεRIβ |
| ASP | CD4 | DAP10 | FcεRIγ |
| ASP | CD4 | DAP10 | DAP10 |
| ASP | CD4 | DAP10 | DAP12 |
| ASP | CD4 | DAP10 | CD32 |
| ASP | CD4 | DAP10 | CD79a |
| ASP | CD4 | DAP10 | CD79b |
| ASP | CD4 | DAP12 | CD8 |
| ASP | CD4 | DAP12 | CD3ζ |
| ASP | CD4 | DAP12 | CD38 |
| ASP | CD4 | DAP12 | CD3γ |
| ASP | CD4 | DAP12 | CD3ε |
| ASP | CD4 | DAP12 | FcyRI-γ |
| ASP | CD4 | DAP12 | FcyRIII-γ |
| ASP | CD4 | DAP12 | FcεRIβ |
| ASP | CD4 | DAP12 | FcεRIγ |
| ASP | CD4 | DAP12 | DAP10 |
| ASP | CD4 | DAP12 | DAP12 |
| ASP | CD4 | DAP12 | CD32 |
| ASP | CD4 | DAP12 | CD79a |
| ASP | CD4 | DAP12 | CD79b |
| ASP | CD4 | MyD88 | CD8 |
| ASP | CD4 | MyD88 | CD3ζ |
| ASP | CD4 | MyD88 | CD38 |
| ASP | CD4 | MyD88 | CD3γ |
| ASP | CD4 | MyD88 | CD3ε |
| ASP | CD4 | MyD88 | FcyRI-γ |
| ASP | CD4 | MyD88 | FcyRIII-γ |
| ASP | CD4 | MyD88 | FcεRIβ |
| ASP | CD4 | MyD88 | FcεRIγ |
| ASP | CD4 | MyD88 | DAP10 |
| ASP | CD4 | MyD88 | DAP12 |
| ASP | CD4 | MyD88 | CD32 |
| ASP | CD4 | MyD88 | CD79a |
| ASP | CD4 | MyD88 | CD79b |
| ASP | CD4 | CD7 | CD8 |
| ASP | CD4 | CD7 | CD3ζ |
| ASP | CD4 | CD7 | CD38 |
| ASP | CD4 | CD7 | CD3γ |
| ASP | CD4 | CD7 | CD3ε |
| ASP | CD4 | CD7 | FcyRI-γ |
| ASP | CD4 | CD7 | FcyRIII-γ |
| ASP | CD4 | CD7 | FcεRIβ |
| ASP | CD4 | CD7 | FcεRIγ |
| ASP | CD4 | CD7 | DAP10 |
| ASP | CD4 | CD7 | DAP12 |
| ASP | CD4 | CD7 | CD32 |
| ASP | CD4 | CD7 | CD79a |
| ASP | CD4 | CD7 | CD79b |
| ASP | CD4 | BTNL3 | CD8 |
| ASP | CD4 | BTNL3 | CD3ζ |
| ASP | CD4 | BTNL3 | CD3δ |
| ASP | CD4 | BTNL3 | CD3γ |
| ASP | CD4 | BTNL3 | CD3ε |
| ASP | CD4 | BTNL3 | FcγRI-γ |
| ASP | CD4 | BTNL3 | FcγRIII-γ |
| ASP | CD4 | BTNL3 | FcεRIβ |
| ASP | CD4 | BTNL3 | FcεRIγ |
| ASP | CD4 | BTNL3 | DAP10 |
| ASP | CD4 | BTNL3 | DAP12 |
| ASP | CD4 | BTNL3 | CD32 |
| ASP | CD4 | BTNL3 | CD79a |
| ASP | CD4 | BTNL3 | CD79b |
| ASP | CD4 | NKG2D | CD8 |
| ASP | CD4 | NKG2D | CD3ζ |
| ASP | CD4 | NKG2D | CD3δ |
| ASP | CD4 | NKG2D | CD3γ |
| ASP | CD4 | NKG2D | CD3ε |
| ASP | CD4 | NKG2D | FcγRI-γ |
| ASP | CD4 | NKG2D | FcγRIII-γ |
| ASP | CD4 | NKG2D | FcεRIβ |
| ASP | CD4 | NKG2D | FcεRIγ |
| ASP | CD4 | NKG2D | DAP10 |
| ASP | CD4 | NKG2D | DAP12 |
| ASP | CD4 | NKG2D | CD32 |
| ASP | CD4 | NKG2D | CD79a |
| ASP | CD4 | NKG2D | CD79b |
| ASP | b2c | CD28 | CD8 |
| ASP | b2c | CD28 | CD3ζ |
| ASP | b2c | CD28 | CD3δ |
| ASP | b2c | CD28 | CD3γ |
| ASP | b2c | CD28 | CD3ε |
| ASP | b2c | CD28 | FcγRI-γ |
| ASP | b2c | CD28 | FcγRIII-γ |
| ASP | b2c | CD28 | FcεRIβ |
| ASP | b2c | CD28 | FcεRIγ |
| ASP | b2c | CD28 | DAP10 |
| ASP | b2c | CD28 | DAP12 |
| ASP | b2c | CD28 | CD32 |
| ASP | b2c | CD28 | CD79a |
| ASP | b2c | CD28 | CD79b |
| ASP | b2c | CD8 | CD8 |
| ASP | b2c | CD8 | CD3ζ |
| ASP | b2c | CD8 | CD3δ |
| ASP | b2c | CD8 | CD3γ |
| ASP | b2c | CD8 | CD3ε |
| ASP | b2c | CD8 | FcγRI-γ |
| ASP | b2c | CD8 | FcγRIII-γ |
| ASP | b2c | CD8 | FcεRIβ |
| ASP | b2c | CD8 | FcεRIγ |
| ASP | b2c | CD8 | DAP10 |
| ASP | b2c | CD8 | DAP12 |
| ASP | b2c | CD8 | CD32 |
| ASP | b2c | CD8 | CD79a |
| ASP | b2c | CD8 | CD79b |
| ASP | b2c | CD4 | CD8 |
| ASP | b2c | CD4 | CD3ζ |
| ASP | b2c | CD4 | CD3δ |
| ASP | b2c | CD4 | CD3γ |
| ASP | b2c | CD4 | CD3ε |
| ASP | b2c | CD4 | FcγRI-γ |
| ASP | b2c | CD4 | FcγRIII-γ |
| ASP | b2c | CD4 | FcεRIβ |
| ASP | b2c | CD4 | FcεRIγ |
| ASP | b2c | CD4 | DAP10 |
| ASP | b2c | CD4 | DAP12 |
| ASP | b2c | CD4 | CD32 |
| ASP | b2c | CD4 | CD79a |
| ASP | b2c | CD4 | CD79b |
| ASP | b2c | b2c | CD8 |
| ASP | b2c | b2c | CD3ζ |
| ASP | b2c | b2c | CD3δ |
| ASP | b2c | b2c | CD3γ |
| ASP | b2c | b2c | CD3ε |
| ASP | b2c | b2c | FcγRI-γ |
| ASP | b2c | b2c | FcγRIII-γ |
| ASP | b2c | b2c | FcεRIβ |
| ASP | b2c | b2c | FcεRIγ |
| ASP | b2c | b2c | DAP10 |
| ASP | b2c | b2c | DAP12 |
| ASP | b2c | b2c | CD32 |
| ASP | b2c | b2c | CD79a |
| ASP | b2c | b2c | CD79b |
| ASP | b2c | CD137/41BB | CD8 |
| ASP | b2c | CD137/41BB | CD3ζ |
| ASP | b2c | CD137/41BB | CD3δ |
| ASP | b2c | CD137/41BB | CD3γ |
| ASP | b2c | CD137/41BB | CD3ε |
| ASP | b2c | CD137/41BB | FcγRI-γ |
| ASP | b2c | CD137/41BB | FcγRIII-γ |
| ASP | b2c | CD137/41BB | FcεRIβ |
| ASP | b2c | CD137/41BB | FcεRIγ |
| ASP | b2c | CD137/41BB | DAP10 |
| ASP | b2c | CD137/41BB | DAP12 |
| ASP | b2c | CD137/41BB | CD32 |
| ASP | b2c | CD137/41BB | CD79a |
| ASP | b2c | CD137/41BB | CD79b |
| ASP | b2c | ICOS | CD8 |
| ASP | b2c | ICOS | CD3ζ |
| ASP | b2c | ICOS | CD3δ |
| ASP | b2c | ICOS | CD3γ |
| ASP | b2c | ICOS | CD3ε |
| ASP | b2c | ICOS | FcγRI-γ |
| ASP | b2c | ICOS | FcγRIII-γ |
| ASP | b2c | ICOS | FcεRIβ |
| ASP | b2c | ICOS | FcεRIγ |
| ASP | b2c | ICOS | DAP10 |
| ASP | b2c | ICOS | DAP12 |
| ASP | b2c | ICOS | CD32 |
| ASP | b2c | ICOS | CD79a |
| ASP | b2c | ICOS | CD79b |
| ASP | b2c | CD27 | CD8 |
| ASP | b2c | CD27 | CD3ζ |
| ASP | b2c | CD27 | CD3δ |
| ASP | b2c | CD27 | CD3γ |
| ASP | b2c | CD27 | CD3ε |
| ASP | b2c | CD27 | FcγRI-γ |
| ASP | b2c | CD27 | FcγRIII-γ |
| ASP | b2c | CD27 | FcεRIβ |
| ASP | b2c | CD27 | FcεRIγ |
| ASP | b2c | CD27 | DAP10 |
| ASP | b2c | CD27 | DAP12 |
| ASP | b2c | CD27 | CD32 |
| ASP | b2c | CD27 | CD79a |
| ASP | b2c | CD27 | CD79b |
| ASP | b2c | CD28δ | CD8 |
| ASP | b2c | CD28δ | CD3ζ |
| ASP | b2c | CD28δ | CD3δ |
| ASP | b2c | CD28δ | CD3γ |
| ASP | b2c | CD28δ | CD3ε |
| ASP | b2c | CD28δ | FcγRI-γ |
| ASP | b2c | CD28δ | FcγRIII-γ |
| ASP | b2c | CD28δ | FcεRIβ |
| ASP | b2c | CD28δ | FcεRIγ |
| ASP | b2c | CD28δ | DAP10 |
| ASP | b2c | CD28δ | DAP12 |
| ASP | b2c | CD28δ | CD32 |
| ASP | b2c | CD28δ | CD79a |
| ASP | b2c | CD28δ | CD79b |
| ASP | b2c | CD80 | CD8 |
| ASP | b2c | CD80 | CD3ζ |
| ASP | b2c | CD80 | CD3δ |
| ASP | b2c | CD80 | CD3γ |
| ASP | b2c | CD80 | CD3ε |
| ASP | b2c | CD80 | FcγRI-γ |
| ASP | b2c | CD80 | FcγRIII-γ |
| ASP | b2c | CD80 | FcεRIβ |
| ASP | b2c | CD80 | FcεRIγ |
| ASP | b2c | CD80 | DAP10 |
| ASP | b2c | CD80 | DAP12 |
| ASP | b2c | CD80 | CD32 |
| ASP | b2c | CD80 | CD79a |
| ASP | b2c | CD80 | CD79b |
| ASP | b2c | CD86 | CD8 |
| ASP | b2c | CD86 | CD3ζ |
| ASP | b2c | CD86 | CD3δ |
| ASP | b2c | CD86 | CD3γ |
| ASP | b2c | CD86 | CD3ε |
| ASP | b2c | CD86 | FcγRI-γ |
| ASP | b2c | CD86 | FcγRIII-γ |
| ASP | b2c | CD86 | FcεRIβ |
| ASP | b2c | CD86 | FcεRIγ |
| ASP | b2c | CD86 | DAP10 |
| ASP | b2c | CD86 | DAP12 |
| ASP | b2c | CD86 | CD32 |
| ASP | b2c | CD86 | CD79a |
| ASP | b2c | CD86 | CD79b |
| ASP | b2c | OX40 | CD8 |
| ASP | b2c | OX40 | CD3ζ |
| ASP | b2c | OX40 | CD3δ |
| ASP | b2c | OX40 | CD3γ |
| ASP | b2c | OX40 | CD3ε |
| ASP | b2c | OX40 | FcγRI-γ |
| ASP | b2c | OX40 | FcγRIII-γ |
| ASP | b2c | OX40 | FcεRIβ |
| ASP | b2c | OX40 | FcεRIγ |
| ASP | b2c | OX40 | DAP10 |
| ASP | b2c | OX40 | DAP12 |
| ASP | b2c | OX40 | CD32 |
| ASP | b2c | OX40 | CD79a |
| ASP | b2c | OX40 | CD79b |
| ASP | b2c | DAP10 | CD8 |
| ASP | b2c | DAP10 | CD3ζ |
| ASP | b2c | DAP10 | CD3δ |
| ASP | b2c | DAP10 | CD3γ |
| ASP | b2c | DAP10 | CD3ε |
| ASP | b2c | DAP10 | FcγRI-γ |
| ASP | b2c | DAP10 | FcγRIII-γ |
| ASP | b2c | DAP10 | FcεRIβ |
| ASP | b2c | DAP10 | FcεRIγ |
| ASP | b2c | DAP10 | DAP10 |
| ASP | b2c | DAP10 | DAP12 |
| ASP | b2c | DAP10 | CD32 |
| ASP | b2c | DAP10 | CD79a |
| ASP | b2c | DAP10 | CD79b |
| ASP | b2c | DAP12 | CD8 |
| ASP | b2c | DAP12 | CD3ζ |
| ASP | b2c | DAP12 | CD3δ |
| ASP | b2c | DAP12 | CD3γ |
| ASP | b2c | DAP12 | CD3ε |
| ASP | b2c | DAP12 | FcγRI-γ |
| ASP | b2c | DAP12 | FcγRIII-γ |
| ASP | b2c | DAP12 | FcεRIβ |
| ASP | b2c | DAP12 | FcεRIγ |
| ASP | b2c | DAP12 | DAP10 |
| ASP | b2c | DAP12 | DAP12 |
| ASP | b2c | DAP12 | CD32 |
| ASP | b2c | DAP12 | CD79a |
| ASP | b2c | DAP12 | CD79b |
| ASP | b2c | MyD88 | CD8 |
| ASP | b2c | MyD88 | CD3ζ |
| ASP | b2c | MyD88 | CD3δ |
| ASP | b2c | MyD88 | CD3γ |
| ASP | b2c | MyD88 | CD3ε |
| ASP | b2c | MyD88 | FcγRI-γ |
| ASP | b2c | MyD88 | FcγRIII-γ |
| ASP | b2c | MyD88 | FcεRIβ |
| ASP | b2c | MyD88 | FcεRIγ |
| ASP | b2c | MyD88 | DAP10 |
| ASP | b2c | MyD88 | DAP12 |
| ASP | b2c | MyD88 | CD32 |
| ASP | b2c | MyD88 | CD79a |
| ASP | b2c | MyD88 | CD79b |
| ASP | b2c | CD7 | CD8 |
| ASP | b2c | CD7 | CD3ζ |
| ASP | b2c | CD7 | CD3δ |
| ASP | b2c | CD7 | CD3γ |
| ASP | b2c | CD7 | CD3ε |
| ASP | b2c | CD7 | FcγRI-γ |
| ASP | b2c | CD7 | FcγRIII-γ |
| ASP | b2c | CD7 | FcεRIβ |
| ASP | b2c | CD7 | FcεRIγ |
| ASP | b2c | CD7 | DAP10 |
| ASP | b2c | CD7 | DAP12 |
| ASP | b2c | CD7 | CD32 |
| ASP | b2c | CD7 | CD79a |
| ASP | b2c | CD7 | CD79b |
| ASP | b2c | BTNL3 | CD8 |
| ASP | b2c | BTNL3 | CD3ζ |
| ASP | b2c | BTNL3 | CD3δ |
| ASP | b2c | BTNL3 | CD3γ |
| ASP | b2c | BTNL3 | CD3ε |
| ASP | b2c | BTNL3 | FcyRI-γ |
| ASP | b2c | BTNL3 | FcyRIII-γ |
| ASP | b2c | BTNL3 | FcεRIβ |
| ASP | b2c | BTNL3 | FcεRIγ |
| ASP | b2c | BTNL3 | DAP10 |
| ASP | b2c | BTNL3 | DAP12 |
| ASP | b2c | BTNL3 | CD32 |
| ASP | b2c | BTNL3 | CD79a |
| ASP | b2c | BTNL3 | CD79b |
| ASP | b2c | NKG2D | CD8 |
| ASP | b2c | NKG2D | CD3ζ |
| ASP | b2c | NKG2D | CD3δ |
| ASP | b2c | NKG2D | CD3γ |
| ASP | b2c | NKG2D | CD3ε |
| ASP | b2c | NKG2D | FcyRI-γ |
| ASP | b2c | NKG2D | FcyRIII-γ |
| ASP | b2c | NKG2D | FcεRIβ |
| ASP | b2c | NKG2D | FcεRIγ |
| ASP | b2c | NKG2D | DAP10 |
| ASP | b2c | NKG2D | DAP12 |
| ASP | b2c | NKG2D | CD32 |
| ASP | b2c | NKG2D | CD79a |
| ASP | b2c | NKG2D | CD79b |
| ASP | CD137/41BB | CD28 | CD8 |
| ASP | CD137/41BB | CD28 | CD3ζ |
| ASP | CD137/41BB | CD28 | CD3δ |
| ASP | CD137/41BB | CD28 | CD3γ |
| ASP | CD137/41BB | CD28 | CD3ε |
| ASP | CD137/41BB | CD28 | FcyRI-γ |
| ASP | CD137/41BB | CD28 | FcyRIII-γ |
| ASP | CD137/41BB | CD28 | FcεRIβ |
| ASP | CD137/41BB | CD28 | FcεRIγ |
| ASP | CD137/41BB | CD28 | DAP10 |
| ASP | CD137/41BB | CD28 | DAP12 |
| ASP | CD137/41BB | CD28 | CD32 |
| ASP | CD137/41BB | CD28 | CD79a |
| ASP | CD137/41BB | CD28 | CD79b |
| ASP | CD137/41BB | CD8 | CD8 |
| ASP | CD137/41BB | CD8 | CD3ζ |
| ASP | CD137/41BB | CD8 | CD3δ |
| ASP | CD137/41BB | CD8 | CD3γ |
| ASP | CD137/41BB | CD8 | CD3ε |
| ASP | CD137/41BB | CD8 | FcyRI-γ |
| ASP | CD137/41BB | CD8 | FcyRIII-γ |
| ASP | CD137/41BB | CD8 | FcεRIβ |
| ASP | CD137/41BB | CD8 | FcεRIγ |
| ASP | CD137/41BB | CD8 | DAP10 |
| ASP | CD137/41BB | CD8 | DAP12 |
| ASP | CD137/41BB | CD8 | CD32 |
| ASP | CD137/41BB | CD8 | CD79a |
| ASP | CD137/41BB | CD8 | CD79b |
| ASP | CD137/41BB | CD4 | CD8 |
| ASP | CD137/41BB | CD4 | CD3ζ |
| ASP | CD137/41BB | CD4 | CD3δ |
| ASP | CD137/41BB | CD4 | CD3γ |
| ASP | CD137/41BB | CD4 | CD3ε |
| ASP | CD137/41BB | CD4 | FcyRI-γ |
| ASP | CD137/41BB | CD4 | FcyRIII-γ |
| ASP | CD137/41BB | CD4 | FcεRIβ |
| ASP | CD137/41BB | CD4 | FcεRIγ |
| ASP | CD137/41BB | CD4 | DAP10 |
| ASP | CD137/41BB | CD4 | DAP12 |
| ASP | CD137/41BB | CD4 | CD32 |
| ASP | CD137/41BB | CD4 | CD79a |
| ASP | CD137/41BB | CD4 | CD79b |
| ASP | CD137/41BB | b2c | CD8 |
| ASP | CD137/41BB | b2c | CD3ζ |
| ASP | CD137/41BB | b2c | CD3δ |
| ASP | CD137/41BB | b2c | CD3γ |
| ASP | CD137/41BB | b2c | CD3ε |
| ASP | CD137/41BB | b2c | FcγRI-γ |
| ASP | CD137/41BB | b2c | FcγRIII-γ |
| ASP | CD137/41BB | b2c | FcεRIβ |
| ASP | CD137/41BB | b2c | FcεRIγ |
| ASP | CD137/41BB | b2c | DAP10 |
| ASP | CD137/41BB | b2c | DAP12 |
| ASP | CD137/41BB | b2c | CD32 |
| ASP | CD137/41BB | b2c | CD79a |
| ASP | CD137/41BB | b2c | CD79b |
| ASP | CD137/41BB | CD137/41BB | CD8 |
| ASP | CD137/41BB | CD137/41BB | CD3ζ |
| ASP | CD137/41BB | CD137/41BB | CD3δ |
| ASP | CD137/41BB | CD137/41BB | CD3γ |
| ASP | CD137/41BB | CD137/41BB | CD3ε |
| ASP | CD137/41BB | CD137/41BB | FcγRI-γ |
| ASP | CD137/41BB | CD137/41BB | FcγRIII-γ |
| ASP | CD137/41BB | CD137/41BB | FcεRIβ |
| ASP | CD137/41BB | CD137/41BB | FcεRIγ |
| ASP | CD137/41BB | CD137/41BB | DAP10 |
| ASP | CD137/41BB | CD137/41BB | DAP12 |
| ASP | CD137/41BB | CD137/41BB | CD32 |
| ASP | CD137/41BB | CD137/41BB | CD79a |
| ASP | CD137/41BB | CD137/41BB | CD79b |
| ASP | CD137/41BB | ICOS | CD8 |
| ASP | CD137/41BB | ICOS | CD3ζ |
| ASP | CD137/41BB | ICOS | CD3δ |
| ASP | CD137/41BB | ICOS | CD3γ |
| ASP | CD137/41BB | ICOS | CD3ε |
| ASP | CD137/41BB | ICOS | FcγRI-γ |
| ASP | CD137/41BB | ICOS | FcγRIII-γ |
| ASP | CD137/41BB | ICOS | FcεRIβ |
| ASP | CD137/41BB | ICOS | FcεRIγ |
| ASP | CD137/41BB | ICOS | DAP10 |
| ASP | CD137/41BB | ICOS | DAP12 |
| ASP | CD137/41BB | ICOS | CD32 |
| ASP | CD137/41BB | ICOS | CD79a |
| ASP | CD137/41BB | ICOS | CD79b |
| ASP | CD137/41BB | CD27 | CD8 |
| ASP | CD137/41BB | CD27 | CD3ζ |
| ASP | CD137/41BB | CD27 | CD3δ |
| ASP | CD137/41BB | CD27 | CD3γ |
| ASP | CD137/41BB | CD27 | CD3ε |
| ASP | CD137/41BB | CD27 | FcγRI-γ |
| ASP | CD137/41BB | CD27 | FcγRIII-γ |
| ASP | CD137/41BB | CD27 | FcεRIβ |
| ASP | CD137/41BB | CD27 | FcεRIγ |
| ASP | CD137/41BB | CD27 | DAP10 |
| ASP | CD137/41BB | CD27 | DAP12 |
| ASP | CD137/41BB | CD27 | CD32 |
| ASP | CD137/41BB | CD27 | CD79a |
| ASP | CD137/41BB | CD27 | CD79b |
| ASP | CD137/41BB | CD28δ | CD8 |
| ASP | CD137/41BB | CD28δ | CD3ζ |
| ASP | CD137/41BB | CD28δ | CD3δ |
| ASP | CD137/41BB | CD28δ | CD3γ |
| ASP | CD137/41BB | CD28δ | CD3ε |
| ASP | CD137/41BB | CD28δ | FcγRI-γ |
| ASP | CD137/41BB | CD28δ | FcγRIII-γ |
| ASP | CD137/41BB | CD28δ | FcεRIβ |
| ASP | CD137/41BB | CD28δ | FcεRIγ |
| ASP | CD137/41BB | CD28δ | DAP10 |
| ASP | CD137/41BB | CD28δ | DAP12 |
| ASP | CD137/41BB | CD28δ | CD32 |
| ASP | CD137/41BB | CD28δ | CD79a |
| ASP | CD137/41BB | CD28δ | CD79b |
| ASP | CD137/41BB | CD80 | CD8 |
| ASP | CD137/41BB | CD80 | CD3ζ |
| ASP | CD137/41BB | CD80 | CD3δ |
| ASP | CD137/41BB | CD80 | CD3γ |
| ASP | CD137/41BB | CD80 | CD3ε |
| ASP | CD137/41BB | CD80 | FcγRI-γ |
| ASP | CD137/41BB | CD80 | FcγRIII-γ |
| ASP | CD137/41BB | CD80 | FcεRIβ |
| ASP | CD137/41BB | CD80 | FcεRIγ |
| ASP | CD137/41BB | CD80 | DAP10 |
| ASP | CD137/41BB | CD80 | DAP12 |
| ASP | CD137/41BB | CD80 | CD32 |
| ASP | CD137/41BB | CD80 | CD79a |
| ASP | CD137/41BB | CD80 | CD79b |
| ASP | CD137/41BB | CD86 | CD8 |
| ASP | CD137/41BB | CD86 | CD3ζ |
| ASP | CD137/41BB | CD86 | CD3δ |
| ASP | CD137/41BB | CD86 | CD3γ |
| ASP | CD137/41BB | CD86 | CD3ε |
| ASP | CD137/41BB | CD86 | FcγRI-γ |
| ASP | CD137/41BB | CD86 | FcγRIII-γ |
| ASP | CD137/41BB | CD86 | FcεRIβ |
| ASP | CD137/41BB | CD86 | FcεRIγ |
| ASP | CD137/41BB | CD86 | DAP10 |
| ASP | CD137/41BB | CD86 | DAP12 |
| ASP | CD137/41BB | CD86 | CD32 |
| ASP | CD137/41BB | CD86 | CD79a |
| ASP | CD137/41BB | CD86 | CD79b |
| ASP | CD137/41BB | OX40 | CD8 |
| ASP | CD137/41BB | OX40 | CD3ζ |
| ASP | CD137/41BB | OX40 | CD3δ |
| ASP | CD137/41BB | OX40 | CD3γ |
| ASP | CD137/41BB | OX40 | CD3ε |
| ASP | CD137/41BB | OX40 | FcγRI-γ |
| ASP | CD137/41BB | OX40 | FcγRIII-γ |
| ASP | CD137/41BB | OX40 | FcεRIβ |
| ASP | CD137/41BB | OX40 | FcεRIγ |
| ASP | CD137/41BB | OX40 | DAP10 |
| ASP | CD137/41BB | OX40 | DAP12 |
| ASP | CD137/41BB | OX40 | CD32 |
| ASP | CD137/41BB | OX40 | CD79a |
| ASP | CD137/41BB | OX40 | CD79b |
| ASP | CD137/41BB | DAP10 | CD8 |
| ASP | CD137/41BB | DAP10 | CD3ζ |
| ASP | CD137/41BB | DAP10 | CD3δ |
| ASP | CD137/41BB | DAP10 | CD3γ |
| ASP | CD137/41BB | DAP10 | CD3ε |
| ASP | CD137/41BB | DAP10 | FcγRI-γ |
| ASP | CD137/41BB | DAP10 | FcγRIII-γ |
| ASP | CD137/41BB | DAP10 | FcεRIβ |
| ASP | CD137/41BB | DAP10 | FcεRIγ |
| ASP | CD137/41BB | DAP10 | DAP10 |
| ASP | CD137/41BB | DAP10 | DAP12 |
| ASP | CD137/41BB | DAP10 | CD32 |
| ASP | CD137/41BB | DAP10 | CD79a |
| ASP | CD137/41BB | DAP10 | CD79b |
| ASP | CD137/41BB | DAP12 | CD8 |
| ASP | CD137/41BB | DAP12 | CD3ζ |
| ASP | CD137/41BB | DAP12 | CD3δ |
| ASP | CD137/41BB | DAP12 | CD3γ |
| ASP | CD137/41BB | DAP12 | CD3ε |
| ASP | CD137/41BB | DAP12 | FcγRI-γ |
| ASP | CD137/41BB | DAP12 | FcγRIII-γ |
| ASP | CD137/41BB | DAP12 | FcεRIβ |
| ASP | CD137/41BB | DAP12 | FcεRIγ |
| ASP | CD137/41BB | DAP12 | DAP10 |
| ASP | CD137/41BB | DAP12 | DAP12 |
| ASP | CD137/41BB | DAP12 | CD32 |
| ASP | CD137/41BB | DAP12 | CD79a |
| ASP | CD137/41BB | DAP12 | CD79b |
| ASP | CD137/41BB | MyD88 | CD8 |
| ASP | CD137/41BB | MyD88 | CD3ζ |
| ASP | CD137/41BB | MyD88 | CD3δ |
| ASP | CD137/41BB | MyD88 | CD3γ |
| ASP | CD137/41BB | MyD88 | CD3ε |
| ASP | CD137/41BB | MyD88 | FcγRI-γ |
| ASP | CD137/41BB | MyD88 | FcγRIII-γ |
| ASP | CD137/41BB | MyD88 | FcεRIβ |
| ASP | CD137/41BB | MyD88 | FcεRIγ |
| ASP | CD137/41BB | MyD88 | DAP10 |
| ASP | CD137/41BB | MyD88 | DAP12 |
| ASP | CD137/41BB | MyD88 | CD32 |
| ASP | CD137/41BB | MyD88 | CD79a |
| ASP | CD137/41BB | MyD88 | CD79b |
| ASP | CD137/41BB | CD7 | CD8 |
| ASP | CD137/41BB | CD7 | CD3ζ |
| ASP | CD137/41BB | CD7 | CD3δ |
| ASP | CD137/41BB | CD7 | CD3γ |
| ASP | CD137/41BB | CD7 | CD3ε |
| ASP | CD137/41BB | CD7 | FcγRI-γ |
| ASP | CD137/41BB | CD7 | FcγRIII-γ |
| ASP | CD137/41BB | CD7 | FcεRIβ |
| ASP | CD137/41BB | CD7 | FcεRIγ |
| ASP | CD137/41BB | CD7 | DAP10 |
| ASP | CD137/41BB | CD7 | DAP12 |
| ASP | CD137/41BB | CD7 | CD32 |
| ASP | CD137/41BB | CD7 | CD79a |
| ASP | CD137/41BB | CD7 | CD79b |
| ASP | CD137/41BB | BTNL3 | CD8 |
| ASP | CD137/41BB | BTNL3 | CD3ζ |
| ASP | CD137/41BB | BTNL3 | CD3δ |
| ASP | CD137/41BB | BTNL3 | CD3γ |
| ASP | CD137/41BB | BTNL3 | CD3ε |
| ASP | CD137/41BB | BTNL3 | FcγRI-γ |
| ASP | CD137/41BB | BTNL3 | FcγRIII-γ |
| ASP | CD137/41BB | BTNL3 | FcεRIβ |
| ASP | CD137/41BB | BTNL3 | FcεRIγ |
| ASP | CD137/41BB | BTNL3 | DAP10 |
| ASP | CD137/41BB | BTNL3 | DAP12 |
| ASP | CD137/41BB | BTNL3 | CD32 |
| ASP | CD137/41BB | BTNL3 | CD79a |
| ASP | CD137/41BB | BTNL3 | CD79b |
| ASP | CD137/41BB | NKG2D | CD8 |
| ASP | CD137/41BB | NKG2D | CD3ζ |
| ASP | CD137/41BB | NKG2D | CD3δ |
| ASP | CD137/41BB | NKG2D | CD3γ |
| ASP | CD137/41BB | NKG2D | CD3ε |
| ASP | CD137/41BB | NKG2D | FcγRI-γ |
| ASP | CD137/41BB | NKG2D | FcγRIII-γ |
| ASP | CD137/41BB | NKG2D | FcεRIβ |
| ASP | CD137/41BB | NKG2D | FcεRIγ |
| ASP | CD137/41BB | NKG2D | DAP10 |
| ASP | CD137/41BB | NKG2D | DAP12 |
| ASP | CD137/41BB | NKG2D | CD32 |
| ASP | CD137/41BB | NKG2D | CD79a |
| ASP | CD137/41BB | NKG2D | CD79b |
| ASP | ICOS | CD28 | CD8 |
| ASP | ICOS | CD28 | CD3ζ |
| ASP | ICOS | CD28 | CD3δ |
| ASP | ICOS | CD28 | CD3γ |
| ASP | ICOS | CD28 | CD3ε |
| ASP | ICOS | CD28 | FcγRI-γ |
| ASP | ICOS | CD28 | FcγRIII-γ |
| ASP | ICOS | CD28 | FcεRIβ |
| ASP | ICOS | CD28 | FcεRIγ |
| ASP | ICOS | CD28 | DAP10 |
| ASP | ICOS | CD28 | DAP12 |
| ASP | ICOS | CD28 | CD32 |
| ASP | ICOS | CD28 | CD79a |
| ASP | ICOS | CD28 | CD79b |
| ASP | ICOS | CD8 | CD8 |
| ASP | ICOS | CD8 | CD3ζ |
| ASP | ICOS | CD8 | CD3δ |
| ASP | ICOS | CD8 | CD3γ |
| ASP | ICOS | CD8 | CD3ε |
| ASP | ICOS | CD8 | FcγRI-γ |
| ASP | ICOS | CD8 | FcγRIII-γ |
| ASP | ICOS | CD8 | FcεRIβ |
| ASP | ICOS | CD8 | FcεRIγ |
| ASP | ICOS | CD8 | DAP10 |
| ASP | ICOS | CD8 | DAP12 |
| ASP | ICOS | CD8 | CD32 |
| ASP | ICOS | CD8 | CD79a |
| ASP | ICOS | CD8 | CD79b |
| ASP | ICOS | CD4 | CD8 |
| ASP | ICOS | CD4 | CD3ζ |
| ASP | ICOS | CD4 | CD3δ |
| ASP | ICOS | CD4 | CD3γ |
| ASP | ICOS | CD4 | CD3ε |
| ASP | ICOS | CD4 | FcγRI-γ |
| ASP | ICOS | CD4 | FcγRIII-γ |
| ASP | ICOS | CD4 | FcεRIβ |
| ASP | ICOS | CD4 | FcεRIγ |
| ASP | ICOS | CD4 | DAP10 |
| ASP | ICOS | CD4 | DAP12 |
| ASP | ICOS | CD4 | CD32 |
| ASP | ICOS | CD4 | CD79a |
| ASP | ICOS | CD4 | CD79b |
| ASP | ICOS | b2c | CD8 |
| ASP | ICOS | b2c | CD3ζ |
| ASP | ICOS | b2c | CD3δ |
| ASP | ICOS | b2c | CD3γ |
| ASP | ICOS | b2c | CD3ε |
| ASP | ICOS | b2c | FcγRI-γ |
| ASP | ICOS | b2c | FcγRIII-γ |
| ASP | ICOS | b2c | FcεRIβ |
| ASP | ICOS | b2c | FcεRIγ |
| ASP | ICOS | b2c | DAP10 |
| ASP | ICOS | b2c | DAP12 |
| ASP | ICOS | b2c | CD32 |
| ASP | ICOS | b2c | CD79a |
| ASP | ICOS | b2c | CD79b |
| ASP | ICOS | CD137/41BB | CD8 |
| ASP | ICOS | CD137/41BB | CD3ζ |
| ASP | ICOS | CD137/41BB | CD3δ |
| ASP | ICOS | CD137/41BB | CD3γ |
| ASP | ICOS | CD137/41BB | CD3ε |
| ASP | ICOS | CD137/41BB | FcγRI-γ |
| ASP | ICOS | CD137/41BB | FcγRIII-γ |
| ASP | ICOS | CD137/41BB | FcεRIβ |
| ASP | ICOS | CD137/41BB | FcεRIγ |
| ASP | ICOS | CD137/41BB | DAP10 |
| ASP | ICOS | CD137/41BB | DAP12 |
| ASP | ICOS | CD137/41BB | CD32 |
| ASP | ICOS | CD137/41BB | CD79a |
| ASP | ICOS | CD137/41BB | CD79b |
| ASP | ICOS | ICOS | CD8 |
| ASP | ICOS | ICOS | CD3ζ |
| ASP | ICOS | ICOS | CD3δ |
| ASP | ICOS | ICOS | CD3γ |
| ASP | ICOS | ICOS | CD3ε |
| ASP | ICOS | ICOS | FcγRI-γ |
| ASP | ICOS | ICOS | FcγRIII-γ |
| ASP | ICOS | ICOS | FcεRIβ |
| ASP | ICOS | ICOS | FcεRIγ |
| ASP | ICOS | ICOS | DAP10 |
| ASP | ICOS | ICOS | DAP12 |
| ASP | ICOS | ICOS | CD32 |
| ASP | ICOS | ICOS | CD79a |
| ASP | ICOS | ICOS | CD79b |
| ASP | ICOS | CD27 | CD8 |
| ASP | ICOS | CD27 | CD3ζ |
| ASP | ICOS | CD27 | CD3δ |
| ASP | ICOS | CD27 | CD3γ |
| ASP | ICOS | CD27 | CD3ε |
| ASP | ICOS | CD27 | FcγRI-γ |
| ASP | ICOS | CD27 | FcγRIII-γ |
| ASP | ICOS | CD27 | FcεRIβ |
| ASP | ICOS | CD27 | FcεRIγ |
| ASP | ICOS | CD27 | DAP10 |
| ASP | ICOS | CD27 | DAP12 |
| ASP | ICOS | CD27 | CD32 |
| ASP | ICOS | CD27 | CD79a |
| ASP | ICOS | CD27 | CD79b |
| ASP | ICOS | CD28δ | CD8 |
| ASP | ICOS | CD28δ | CD3ζ |
| ASP | ICOS | CD28δ | CD3δ |
| ASP | ICOS | CD28δ | CD3γ |
| ASP | ICOS | CD28δ | CD3ε |
| ASP | ICOS | CD28δ | FcγRI-γ |
| ASP | ICOS | CD28δ | FcγRIII-γ |
| ASP | ICOS | CD28δ | FcεRIβ |
| ASP | ICOS | CD28δ | FcεRIγ |
| ASP | ICOS | CD28δ | DAP10 |
| ASP | ICOS | CD28δ | DAP12 |
| ASP | ICOS | CD28δ | CD32 |
| ASP | ICOS | CD28δ | CD79a |
| ASP | ICOS | CD28δ | CD79b |
| ASP | ICOS | CD80 | CD8 |
| ASP | ICOS | CD80 | CD3ζ |
| ASP | ICOS | CD80 | CD3δ |
| ASP | ICOS | CD80 | CD3γ |
| ASP | ICOS | CD80 | CD3ε |
| ASP | ICOS | CD80 | FcγRI-γ |
| ASP | ICOS | CD80 | FcγRIII-γ |
| ASP | ICOS | CD80 | FcεRIβ |
| ASP | ICOS | CD80 | FcεRIγ |
| ASP | ICOS | CD80 | DAP10 |
| ASP | ICOS | CD80 | DAP12 |
| ASP | ICOS | CD80 | CD32 |
| ASP | ICOS | CD80 | CD79a |
| ASP | ICOS | CD80 | CD79b |
| ASP | ICOS | CD86 | CD8 |
| ASP | ICOS | CD86 | CD3ζ |
| ASP | ICOS | CD86 | CD3δ |
| ASP | ICOS | CD86 | CD3γ |
| ASP | ICOS | CD86 | CD3ε |
| ASP | ICOS | CD86 | FcγRI-γ |
| ASP | ICOS | CD86 | FcγRIII-γ |
| ASP | ICOS | CD86 | FcεRIβ |
| ASP | ICOS | CD86 | FcεRIγ |
| ASP | ICOS | CD86 | DAP10 |
| ASP | ICOS | CD86 | DAP12 |
| ASP | ICOS | CD86 | CD32 |
| ASP | ICOS | CD86 | CD79a |
| ASP | ICOS | CD86 | CD79b |
| ASP | ICOS | OX40 | CD8 |
| ASP | ICOS | OX40 | CD3ζ |
| ASP | ICOS | OX40 | CD3δ |
| ASP | ICOS | OX40 | CD3γ |
| ASP | ICOS | OX40 | CD3ε |
| ASP | ICOS | OX40 | FcγRI-γ |
| ASP | ICOS | OX40 | FcγRIII-γ |
| ASP | ICOS | OX40 | FcεRIβ |
| ASP | ICOS | OX40 | FcεRIγ |
| ASP | ICOS | OX40 | DAP10 |
| ASP | ICOS | OX40 | DAP12 |
| ASP | ICOS | OX40 | CD32 |
| ASP | ICOS | OX40 | CD79a |
| ASP | ICOS | OX40 | CD79b |
| ASP | ICOS | DAP10 | CD8 |
| ASP | ICOS | DAP10 | CD3ζ |
| ASP | ICOS | DAP10 | CD3δ |
| ASP | ICOS | DAP10 | CD3γ |
| ASP | ICOS | DAP10 | CD3ε |
| ASP | ICOS | DAP10 | FcγRI-γ |
| ASP | ICOS | DAP10 | FcγRIII-γ |
| ASP | ICOS | DAP10 | FcεRIβ |
| ASP | ICOS | DAP10 | FcεRIγ |
| ASP | ICOS | DAP10 | DAP10 |
| ASP | ICOS | DAP10 | DAP12 |
| ASP | ICOS | DAP10 | CD32 |
| ASP | ICOS | DAP10 | CD79a |
| ASP | ICOS | DAP10 | CD79b |
| ASP | ICOS | DAP12 | CD8 |
| ASP | ICOS | DAP12 | CD3ζ |
| ASP | ICOS | DAP12 | CD3δ |
| ASP | ICOS | DAP12 | CD3γ |
| ASP | ICOS | DAP12 | CD3ε |
| ASP | ICOS | DAP12 | FcγRI-γ |
| ASP | ICOS | DAP12 | FcγRIII-γ |
| ASP | ICOS | DAP12 | FcεRIβ |
| ASP | ICOS | DAP12 | FcεRIγ |
| ASP | ICOS | DAP12 | DAP10 |
| ASP | ICOS | DAP12 | DAP12 |
| ASP | ICOS | DAP12 | CD32 |
| ASP | ICOS | DAP12 | CD79a |
| ASP | ICOS | DAP12 | CD79b |
| ASP | ICOS | MyD88 | CD8 |
| ASP | ICOS | MyD88 | CD3ζ |
| ASP | ICOS | MyD88 | CD3δ |
| ASP | ICOS | MyD88 | CD3γ |
| ASP | ICOS | MyD88 | CD3ε |
| ASP | ICOS | MyD88 | FcγRI-γ |
| ASP | ICOS | MyD88 | FcγRIII-γ |
| ASP | ICOS | MyD88 | FcεRIβ |
| ASP | ICOS | MyD88 | FcεRIγ |
| ASP | ICOS | MyD88 | DAP10 |
| ASP | ICOS | MyD88 | DAP12 |
| ASP | ICOS | MyD88 | CD32 |
| ASP | ICOS | MyD88 | CD79a |
| ASP | ICOS | MyD88 | CD79b |
| ASP | ICOS | CD7 | CD8 |
| ASP | ICOS | CD7 | CD3ζ |
| ASP | ICOS | CD7 | CD3δ |
| ASP | ICOS | CD7 | CD3γ |
| ASP | ICOS | CD7 | CD3ε |
| ASP | ICOS | CD7 | FcγRI-γ |
| ASP | ICOS | CD7 | FcγRIII-γ |
| ASP | ICOS | CD7 | FcεRIβ |
| ASP | ICOS | CD7 | FcεRIγ |
| ASP | ICOS | CD7 | DAP10 |
| ASP | ICOS | CD7 | DAP12 |
| ASP | ICOS | CD7 | CD32 |
| ASP | ICOS | CD7 | CD79a |
| ASP | ICOS | CD7 | CD79b |
| ASP | ICOS | BTNL3 | CD8 |
| ASP | ICOS | BTNL3 | CD3ζ |
| ASP | ICOS | BTNL3 | CD3δ |
| ASP | ICOS | BTNL3 | CD3γ |
| ASP | ICOS | BTNL3 | CD3ε |
| ASP | ICOS | BTNL3 | FcγRI-γ |
| ASP | ICOS | BTNL3 | FcγRIII-γ |
| ASP | ICOS | BTNL3 | FcεRIβ |
| ASP | ICOS | BTNL3 | FcεRIγ |
| ASP | ICOS | BTNL3 | DAP10 |
| ASP | ICOS | BTNL3 | DAP12 |
| ASP | ICOS | BTNL3 | CD32 |
| ASP | ICOS | BTNL3 | CD79a |
| ASP | ICOS | BTNL3 | CD79b |
| ASP | ICOS | NKG2D | CD8 |
| ASP | ICOS | NKG2D | CD3ζ |
| ASP | ICOS | NKG2D | CD3δ |
| ASP | ICOS | NKG2D | CD3γ |
| ASP | ICOS | NKG2D | CD3ε |
| ASP | ICOS | NKG2D | FcγRI-γ |
| ASP | ICOS | NKG2D | FcγRIII-γ |
| ASP | ICOS | NKG2D | FcεRIβ |
| ASP | ICOS | NKG2D | FcεRIγ |
| ASP | ICOS | NKG2D | DAP10 |
| ASP | ICOS | NKG2D | DAP12 |
| ASP | ICOS | NKG2D | CD32 |
| ASP | ICOS | NKG2D | CD79a |
| ASP | ICOS | NKG2D | CD79b |
| ASP | CD27 | CD28 | CD8 |
| ASP | CD27 | CD28 | CD3ζ |
| ASP | CD27 | CD28 | CD3δ |
| ASP | CD27 | CD28 | CD3γ |
| ASP | CD27 | CD28 | CD3ε |
| ASP | CD27 | CD28 | FcγRI-γ |
| ASP | CD27 | CD28 | FcγRIII-γ |
| ASP | CD27 | CD28 | FcεRIβ |
| ASP | CD27 | CD28 | FcεRIγ |
| ASP | CD27 | CD28 | DAP10 |
| ASP | CD27 | CD28 | DAP12 |
| ASP | CD27 | CD28 | CD32 |
| ASP | CD27 | CD28 | CD79a |
| ASP | CD27 | CD28 | CD79b |
| ASP | CD27 | CD8 | CD8 |
| ASP | CD27 | CD8 | CD3ζ |
| ASP | CD27 | CD8 | CD3δ |
| ASP | CD27 | CD8 | CD3γ |
| ASP | CD27 | CD8 | CD3ε |
| ASP | CD27 | CD8 | FcγRI-γ |
| ASP | CD27 | CD8 | FcγRIII-γ |
| ASP | CD27 | CD8 | FcεRIβ |
| ASP | CD27 | CD8 | FcεRIγ |
| ASP | CD27 | CD8 | DAP10 |
| ASP | CD27 | CD8 | DAP12 |
| ASP | CD27 | CD8 | CD32 |
| ASP | CD27 | CD8 | CD79a |
| ASP | CD27 | CD8 | CD79b |
| ASP | CD27 | CD4 | CD8 |
| ASP | CD27 | CD4 | CD3ζ |
| ASP | CD27 | CD4 | CD3δ |
| ASP | CD27 | CD4 | CD3γ |
| ASP | CD27 | CD4 | CD3ε |
| ASP | CD27 | CD4 | FcγRI-γ |
| ASP | CD27 | CD4 | FcγRIII-γ |
| ASP | CD27 | CD4 | FcεRIβ |
| ASP | CD27 | CD4 | FcεRIγ |
| ASP | CD27 | CD4 | DAP10 |
| ASP | CD27 | CD4 | DAP12 |
| ASP | CD27 | CD4 | CD32 |
| ASP | CD27 | CD4 | CD79a |
| ASP | CD27 | CD4 | CD79b |
| ASP | CD27 | b2c | CD8 |
| ASP | CD27 | b2c | CD3ζ |
| ASP | CD27 | b2c | CD3δ |
| ASP | CD27 | b2c | CD3γ |
| ASP | CD27 | b2c | CD3ε |
| ASP | CD27 | b2c | FcγRI-γ |
| ASP | CD27 | b2c | FcγRIII-γ |
| ASP | CD27 | b2c | FcεRIβ |
| ASP | CD27 | b2c | FcεRIγ |
| ASP | CD27 | b2c | DAP10 |
| ASP | CD27 | b2c | DAP12 |
| ASP | CD27 | b2c | CD32 |
| ASP | CD27 | b2c | CD79a |
| ASP | CD27 | b2c | CD79b |
| ASP | CD27 | CD 137/41BB | CD8 |
| ASP | CD27 | CD137/41BB | CD3ζ |
| ASP | CD27 | CD137/41BB | CD3δ |
| ASP | CD27 | CD137/41BB | CD3γ |
| ASP | CD27 | CD137/41BB | CD3ε |
| ASP | CD27 | CD137/41BB | FcγRI-γ |
| ASP | CD27 | CD137/41BB | FcγRIII-γ |
| ASP | CD27 | CD137/41BB | FcεRIβ |
| ASP | CD27 | CD137/41BB | FcεRIγ |
| ASP | CD27 | CD137/41BB | DAP10 |
| ASP | CD27 | CD137/41BB | DAP12 |
| ASP | CD27 | CD137/41BB | CD32 |
| ASP | CD27 | CD137/41BB | CD79a |
| ASP | CD27 | CD137/41BB | CD79b |
| ASP | CD27 | ICOS | CD8 |
| ASP | CD27 | ICOS | CD3ζ |
| ASP | CD27 | ICOS | CD3δ |
| ASP | CD27 | ICOS | CD3γ |
| ASP | CD27 | ICOS | CD3ε |
| ASP | CD27 | ICOS | FcγRI-γ |
| ASP | CD27 | ICOS | FcγRIII-γ |
| ASP | CD27 | ICOS | FcεRIβ |
| ASP | CD27 | ICOS | FcεRIγ |
| ASP | CD27 | ICOS | DAP10 |
| ASP | CD27 | ICOS | DAP12 |
| ASP | CD27 | ICOS | CD32 |
| ASP | CD27 | ICOS | CD79a |
| ASP | CD27 | ICOS | CD79b |
| ASP | CD27 | CD27 | CD8 |
| ASP | CD27 | CD27 | CD3ζ |
| ASP | CD27 | CD27 | CD3δ |
| ASP | CD27 | CD27 | CD3γ |
| ASP | CD27 | CD27 | CD3ε |
| ASP | CD27 | CD27 | FcγRI-γ |
| ASP | CD27 | CD27 | FcγRIII-γ |
| ASP | CD27 | CD27 | FcεRIβ |
| ASP | CD27 | CD27 | FcεRIγ |
| ASP | CD27 | CD27 | DAP10 |
| ASP | CD27 | CD27 | DAP12 |
| ASP | CD27 | CD27 | CD32 |
| ASP | CD27 | CD27 | CD79a |
| ASP | CD27 | CD27 | CD79b |
| ASP | CD27 | CD28δ | CD8 |
| ASP | CD27 | CD28δ | CD3ζ |
| ASP | CD27 | CD28δ | CD3δ |
| ASP | CD27 | CD28δ | CD3γ |
| ASP | CD27 | CD28δ | CD3ε |
| ASP | CD27 | CD28δ | FcγRI-γ |
| ASP | CD27 | CD28δ | FcγRIII-γ |
| ASP | CD27 | CD28δ | FcεRIβ |
| ASP | CD27 | CD28δ | FcεRIγ |
| ASP | CD27 | CD28δ | DAP10 |
| ASP | CD27 | CD28δ | DAP12 |
| ASP | CD27 | CD28δ | CD32 |
| ASP | CD27 | CD28δ | CD79a |
| ASP | CD27 | CD28δ | CD79b |
| ASP | CD27 | CD80 | CD8 |
| ASP | CD27 | CD80 | CD3ζ |
| ASP | CD27 | CD80 | CD3δ |
| ASP | CD27 | CD80 | CD3γ |
| ASP | CD27 | CD80 | CD3ε |
| ASP | CD27 | CD80 | FcγRI-γ |
| ASP | CD27 | CD80 | FcγRIII-γ |
| ASP | CD27 | CD80 | FcεRIβ |
| ASP | CD27 | CD80 | FcεRIγ |
| ASP | CD27 | CD80 | DAP10 |
| ASP | CD27 | CD80 | DAP12 |
| ASP | CD27 | CD80 | CD32 |
| ASP | CD27 | CD80 | CD79a |
| ASP | CD27 | CD80 | CD79b |
| ASP | CD27 | CD86 | CD8 |
| ASP | CD27 | CD86 | CD3ζ |
| ASP | CD27 | CD86 | CD3δ |
| ASP | CD27 | CD86 | CD3γ |
| ASP | CD27 | CD86 | CD3ε |
| ASP | CD27 | CD86 | FcγRI-γ |
| ASP | CD27 | CD86 | FcγRIII-γ |
| ASP | CD27 | CD86 | FcεRIβ |
| ASP | CD27 | CD86 | FcεRIγ |
| ASP | CD27 | CD86 | DAP10 |
| ASP | CD27 | CD86 | DAP12 |
| ASP | CD27 | CD86 | CD32 |
| ASP | CD27 | CD86 | CD79a |
| ASP | CD27 | CD86 | CD79b |
| ASP | CD27 | OX40 | CD8 |
| ASP | CD27 | OX40 | CD3ζ |
| ASP | CD27 | OX40 | CD3δ |
| ASP | CD27 | OX40 | CD3γ |
| ASP | CD27 | OX40 | CD3ε |
| ASP | CD27 | OX40 | FcγRI-γ |
| ASP | CD27 | OX40 | FcγRIII-γ |
| ASP | CD27 | OX40 | FcεRIβ |
| ASP | CD27 | OX40 | FcεRIγ |
| ASP | CD27 | OX40 | DAP10 |
| ASP | CD27 | OX40 | DAP12 |
| ASP | CD27 | OX40 | CD32 |
| ASP | CD27 | OX40 | CD79a |
| ASP | CD27 | OX40 | CD79b |
| ASP | CD27 | DAP10 | CD8 |
| ASP | CD27 | DAP10 | CD3ζ |
| ASP | CD27 | DAP10 | CD3δ |
| ASP | CD27 | DAP10 | CD3γ |
| ASP | CD27 | DAP10 | CD3ε |
| ASP | CD27 | DAP10 | FcγRI-γ |
| ASP | CD27 | DAP10 | FcγRIII-γ |
| ASP | CD27 | DAP10 | FcεRIβ |
| ASP | CD27 | DAP10 | FcεRIγ |
| ASP | CD27 | DAP10 | DAP10 |
| ASP | CD27 | DAP10 | DAP12 |
| ASP | CD27 | DAP10 | CD32 |
| ASP | CD27 | DAP10 | CD79a |
| ASP | CD27 | DAP10 | CD79b |
| ASP | CD27 | DAP12 | CD8 |
| ASP | CD27 | DAP12 | CD3ζ |
| ASP | CD27 | DAP12 | CD3δ |
| ASP | CD27 | DAP12 | CD3γ |
| ASP | CD27 | DAP12 | CD3ε |
| ASP | CD27 | DAP12 | FcγRI-γ |
| ASP | CD27 | DAP12 | FcγRIII-γ |
| ASP | CD27 | DAP12 | FcεRIβ |
| ASP | CD27 | DAP12 | FcεRIγ |
| ASP | CD27 | DAP12 | DAP10 |
| ASP | CD27 | DAP12 | DAP12 |
| ASP | CD27 | DAP12 | CD32 |
| ASP | CD27 | DAP12 | CD79a |
| ASP | CD27 | DAP12 | CD79b |
| ASP | CD27 | MyD88 | CD8 |
| ASP | CD27 | MyD88 | CD3ζ |
| ASP | CD27 | MyD88 | CD3δ |
| ASP | CD27 | MyD88 | CD3γ |
| ASP | CD27 | MyD88 | CD3ε |
| ASP | CD27 | MyD88 | FcγRI-γ |
| ASP | CD27 | MyD88 | FcγRIII-γ |
| ASP | CD27 | MyD88 | FcεRIβ |
| ASP | CD27 | MyD88 | FcεRIγ |
| ASP | CD27 | MyD88 | DAP10 |
| ASP | CD27 | MyD88 | DAP12 |
| ASP | CD27 | MyD88 | CD32 |
| ASP | CD27 | MyD88 | CD79a |
| ASP | CD27 | MyD88 | CD79b |
| ASP | CD27 | CD7 | CD8 |
| ASP | CD27 | CD7 | CD3ζ |
| ASP | CD27 | CD7 | CD3δ |
| ASP | CD27 | CD7 | CD3γ |
| ASP | CD27 | CD7 | CD3ε |
| ASP | CD27 | CD7 | FcγRI-γ |
| ASP | CD27 | CD7 | FcγRIII-γ |
| ASP | CD27 | CD7 | FcεRIβ |
| ASP | CD27 | CD7 | FcεRIγ |
| ASP | CD27 | CD7 | DAP10 |
| ASP | CD27 | CD7 | DAP12 |
| ASP | CD27 | CD7 | CD32 |
| ASP | CD27 | CD7 | CD79a |
| ASP | CD27 | CD7 | CD79b |
| ASP | CD27 | BTNL3 | CD8 |
| ASP | CD27 | BTNL3 | CD3ζ |
| ASP | CD27 | BTNL3 | CD3δ |
| ASP | CD27 | BTNL3 | CD3γ |
| ASP | CD27 | BTNL3 | CD3ε |
| ASP | CD27 | BTNL3 | FcγRI-γ |
| ASP | CD27 | BTNL3 | FcγRIII-γ |
| ASP | CD27 | BTNL3 | FcεRIβ |
| ASP | CD27 | BTNL3 | FcεRIγ |
| ASP | CD27 | BTNL3 | DAP10 |
| ASP | CD27 | BTNL3 | DAP12 |
| ASP | CD27 | BTNL3 | CD32 |
| ASP | CD27 | BTNL3 | CD79a |
| ASP | CD27 | BTNL3 | CD79b |
| ASP | CD27 | NKG2D | CD8 |
| ASP | CD27 | NKG2D | CD3ζ |
| ASP | CD27 | NKG2D | CD3δ |
| ASP | CD27 | NKG2D | CD3γ |
| ASP | CD27 | NKG2D | CD3ε |
| ASP | CD27 | NKG2D | FcγRI-γ |
| ASP | CD27 | NKG2D | FcγRIII-γ |
| ASP | CD27 | NKG2D | FcεRIβ |
| ASP | CD27 | NKG2D | FcεRIγ |
| ASP | CD27 | NKG2D | DAP10 |
| ASP | CD27 | NKG2D | DAP12 |
| ASP | CD27 | NKG2D | CD32 |
| ASP | CD27 | NKG2D | CD79a |
| ASP | CD27 | NKG2D | CD79b |
| ASP | CD28δ | CD28 | CD8 |
| ASP | CD28δ | CD28 | CD3ζ |
| ASP | CD28δ | CD28 | CD3δ |
| ASP | CD28δ | CD28 | CD3γ |
| ASP | CD28δ | CD28 | CD3ε |
| ASP | CD28δ | CD28 | FcγRI-γ |
| ASP | CD28δ | CD28 | FcγRIII-γ |
| ASP | CD28δ | CD28 | FcεRIβ |
| ASP | CD28δ | CD28 | FcεRIγ |
| ASP | CD28δ | CD28 | DAP10 |
| ASP | CD28δ | CD28 | DAP12 |
| ASP | CD28δ | CD28 | CD32 |
| ASP | CD28δ | CD28 | CD79a |
| ASP | CD28δ | CD28 | CD79b |
| ASP | CD28δ | CD8 | CD8 |
| ASP | CD28δ | CD8 | CD3ζ |
| ASP | CD28δ | CD8 | CD3δ |
| ASP | CD28δ | CD8 | CD3γ |
| ASP | CD28δ | CD8 | CD3ε |
| ASP | CD28δ | CD8 | FcγRI-γ |
| ASP | CD28δ | CD8 | FcγRIII-γ |
| ASP | CD28δ | CD8 | FcεRIβ |
| ASP | CD28δ | CD8 | FcεRIγ |
| ASP | CD28δ | CD8 | DAP10 |
| ASP | CD28δ | CD8 | DAP12 |
| ASP | CD28δ | CD8 | CD32 |
| ASP | CD28δ | CD8 | CD79a |
| ASP | CD28δ | CD8 | CD79b |
| ASP | CD28δ | CD4 | CD8 |
| ASP | CD28δ | CD4 | CD3ζ |
| ASP | CD28δ | CD4 | CD3δ |
| ASP | CD28δ | CD4 | CD3γ |
| ASP | CD28δ | CD4 | CD3ε |
| ASP | CD28δ | CD4 | FcγRI-γ |
| ASP | CD28δ | CD4 | FcγRIII-γ |
| ASP | CD28δ | CD4 | FcεRIβ |
| ASP | CD28δ | CD4 | FcεRIγ |
| ASP | CD28δ | CD4 | DAP10 |
| ASP | CD28δ | CD4 | DAP12 |
| ASP | CD28δ | CD4 | CD32 |
| ASP | CD28δ | CD4 | CD79a |
| ASP | CD28δ | CD4 | CD79b |
| ASP | CD28b | b2c | CD8 |
| ASP | CD28δ | b2c | CD3ζ |
| ASP | CD28b | b2c | CD3δ |
| ASP | CD28b | b2c | CD3γ |
| ASP | CD28δ | b2c | CD3ε |
| ASP | CD28δ | b2c | FcγRI-γ |
| ASP | CD28b | b2c | FcγRIII-γ |
| ASP | CD28b | b2c | FcεRIβ |
| ASP | CD28δ | b2c | FcεRIγ |
| ASP | CD28δ | b2c | DAP10 |
| ASP | CD28δ | b2c | DAP12 |
| ASP | CD28b | b2c | CD32 |
| ASP | CD28δ | b2c | CD79a |
| ASP | CD28b | b2c | CD79b |
| ASP | CD28δ | CD137/41BB | CD8 |
| ASP | CD28δ | CD137/41BB | CD3ζ |
| ASP | CD28δ | CD137/41BB | CD3δ |
| ASP | CD28δ | CD137/41BB | CD3γ |
| ASP | CD28δ | CD137/41BB | CD3ε |
| ASP | CD28δ | CD137/41BB | FcγRI-γ |
| ASP | CD28δ | CD137/41BB | FcγRIII-γ |
| ASP | CD28δ | CD137/41BB | FcεRIβ |
| ASP | CD28δ | CD137/41BB | FcεRIγ |
| ASP | CD28δ | CD137/41BB | DAP10 |
| ASP | CD28δ | CD137/41BB | DAP12 |
| ASP | CD28δ | CD137/41BB | CD32 |
| ASP | CD28δ | CD137/41BB | CD79a |
| ASP | CD28δ | CD137/41BB | CD79b |
| ASP | CD28δ | ICOS | CD8 |
| ASP | CD28δ | ICOS | CD3ζ |
| ASP | CD28δ | ICOS | CD3δ |
| ASP | CD28δ | ICOS | CD3γ |
| ASP | CD28δ | ICOS | CD3ε |
| ASP | CD28δ | ICOS | FcγRI-γ |
| ASP | CD28δ | ICOS | FcγRIII-γ |
| ASP | CD28δ | ICOS | FcεRIβ |
| ASP | CD28δ | ICOS | FcεRIγ |
| ASP | CD28δ | ICOS | DAP10 |
| ASP | CD28δ | ICOS | DAP12 |
| ASP | CD28δ | ICOS | CD32 |
| ASP | CD28δ | ICOS | CD79a |
| ASP | CD28δ | ICOS | CD79b |
| ASP | CD28δ | CD27 | CD8 |
| ASP | CD28δ | CD27 | CD3ζ |
| ASP | CD28δ | CD27 | CD3δ |
| ASP | CD28δ | CD27 | CD3γ |
| ASP | CD28δ | CD27 | CD3ε |
| ASP | CD28δ | CD27 | FcγRI-γ |
| ASP | CD28δ | CD27 | FcγRIII-γ |
| ASP | CD28δ | CD27 | FcεRIβ |
| ASP | CD28δ | CD27 | FcεRIγ |
| ASP | CD28δ | CD27 | DAP10 |
| ASP | CD28δ | CD27 | DAP12 |
| ASP | CD28δ | CD27 | CD32 |
| ASP | CD28δ | CD27 | CD79a |
| ASP | CD28δ | CD27 | CD79b |
| ASP | CD28δ | CD28δ | CD8 |
| ASP | CD28δ | CD28δ | CD3ζ |
| ASP | CD28δ | CD28δ | CD3δ |
| ASP | CD28δ | CD28δ | CD3γ |
| ASP | CD28δ | CD28δ | CD3ε |
| ASP | CD28δ | CD28δ | FcγRI-γ |
| ASP | CD28δ | CD28δ | FcγRIII-γ |
| ASP | CD28δ | CD28δ | FcεRIβ |
| ASP | CD28δ | CD28δ | FcεRIγ |
| ASP | CD28δ | CD28δ | DAP10 |
| ASP | CD28δ | CD28δ | DAP12 |
| ASP | CD28δ | CD28δ | CD32 |
| ASP | CD28δ | CD28δ | CD79a |
| ASP | CD28δ | CD28δ | CD79b |
| ASP | CD28δ | CD80 | CD8 |
| ASP | CD28δ | CD80 | CD3ζ |
| ASP | CD28δ | CD80 | CD3δ |
| ASP | CD28δ | CD80 | CD3γ |
| ASP | CD28δ | CD80 | CD3ε |
| ASP | CD28δ | CD80 | FcγRI-γ |
| ASP | CD28δ | CD80 | FcγRIII-γ |
| ASP | CD28δ | CD80 | FcεRIβ |
| ASP | CD28δ | CD80 | FcεRIγ |
| ASP | CD28δ | CD80 | DAP10 |
| ASP | CD28δ | CD80 | DAP12 |
| ASP | CD28δ | CD80 | CD32 |
| ASP | CD28δ | CD80 | CD79a |
| ASP | CD28δ | CD80 | CD79b |
| ASP | CD28δ | CD86 | CD8 |
| ASP | CD28δ | CD86 | CD3ζ |
| ASP | CD28δ | CD86 | CD3δ |
| ASP | CD28δ | CD86 | CD3γ |
| ASP | CD28δ | CD86 | CD3ε |
| ASP | CD28δ | CD86 | FcγRI-γ |
| ASP | CD28δ | CD86 | FcγRIII-γ |
| ASP | CD28δ | CD86 | FcεRIβ |
| ASP | CD28δ | CD86 | FcεRIγ |
| ASP | CD28δ | CD86 | DAP10 |
| ASP | CD28δ | CD86 | DAP12 |
| ASP | CD28δ | CD86 | CD32 |
| ASP | CD28δ | CD86 | CD79a |
| ASP | CD28δ | CD86 | CD79b |
| ASP | CD28δ | OX40 | CD8 |
| ASP | CD28δ | OX40 | CD3ζ |
| ASP | CD28δ | OX40 | CD3δ |
| ASP | CD28δ | OX40 | CD3γ |
| ASP | CD28δ | OX40 | CD3ε |
| ASP | CD28δ | OX40 | FcγRI-γ |
| ASP | CD28δ | OX40 | FcγRIII-γ |
| ASP | CD28δ | OX40 | FcεRIβ |
| ASP | CD28δ | OX40 | FcεRIγ |
| ASP | CD28δ | OX40 | DAP10 |
| ASP | CD28δ | OX40 | DAP12 |
| ASP | CD28δ | OX40 | CD32 |
| ASP | CD28δ | OX40 | CD79a |
| ASP | CD28δ | OX40 | CD79b |
| ASP | CD28δ | DAP10 | CD8 |
| ASP | CD28δ | DAP10 | CD3ζ |
| ASP | CD28δ | DAP10 | CD3δ |
| ASP | CD28δ | DAP10 | CD3γ |
| ASP | CD28δ | DAP10 | CD3ε |
| ASP | CD28δ | DAP10 | FcγRI-γ |
| ASP | CD28δ | DAP10 | FcγRIII-γ |
| ASP | CD28δ | DAP10 | FcεRIβ |
| ASP | CD28δ | DAP10 | FcεRIγ |
| ASP | CD28δ | DAP10 | DAP10 |
| ASP | CD28δ | DAP10 | DAP12 |
| ASP | CD28δ | DAP10 | CD32 |
| ASP | CD28δ | DAP10 | CD79a |
| ASP | CD28δ | DAP10 | CD79b |
| ASP | CD28δ | DAP12 | CD8 |
| ASP | CD28δ | DAP12 | CD3ζ |
| ASP | CD28δ | DAP12 | CD3δ |
| ASP | CD28δ | DAP12 | CD3γ |
| ASP | CD28δ | DAP12 | CD3ε |
| ASP | CD28δ | DAP12 | FcγRI-γ |
| ASP | CD28δ | DAP12 | FcγRIII-γ |
| ASP | CD28δ | DAP12 | FcεRIβ |
| ASP | CD28δ | DAP12 | FcεRIγ |
| ASP | CD28δ | DAP12 | DAP10 |
| ASP | CD28δ | DAP12 | DAP12 |
| ASP | CD28δ | DAP12 | CD32 |
| ASP | CD28δ | DAP12 | CD79a |
| ASP | CD28δ | DAP12 | CD79b |
| ASP | CD28δ | MyD88 | CD8 |
| ASP | CD28δ | MyD88 | CD3ζ |
| ASP | CD28δ | MyD88 | CD3δ |
| ASP | CD28δ | MyD88 | CD3γ |
| ASP | CD28δ | MyD88 | CD3ε |
| ASP | CD28δ | MyD88 | FcγRI-γ |
| ASP | CD28δ | MyD88 | FcγRIII-γ |
| ASP | CD28δ | MyD88 | FcεRIβ |
| ASP | CD28δ | MyD88 | FcεRIγ |
| ASP | CD28δ | MyD88 | DAP10 |
| ASP | CD28δ | MyD88 | DAP12 |
| ASP | CD28δ | MyD88 | CD32 |
| ASP | CD28δ | MyD88 | CD79a |
| ASP | CD28δ | MyD88 | CD79b |
| ASP | CD28δ | CD7 | CD8 |
| ASP | CD28δ | CD7 | CD3ζ |
| ASP | CD28δ | CD7 | CD3δ |
| ASP | CD28δ | CD7 | CD3γ |
| ASP | CD28δ | CD7 | CD3ε |
| ASP | CD28δ | CD7 | FcγRI-γ |
| ASP | CD28δ | CD7 | FcγRIII-γ |
| ASP | CD28δ | CD7 | FcεRIβ |
| ASP | CD28δ | CD7 | FcεRIγ |
| ASP | CD28δ | CD7 | DAP10 |
| ASP | CD28δ | CD7 | DAP12 |
| ASP | CD28δ | CD7 | CD32 |
| ASP | CD28δ | CD7 | CD79a |
| ASP | CD28δ | CD7 | CD79b |
| ASP | CD28δ | BTNL3 | CD8 |
| ASP | CD28δ | BTNL3 | CD3ζ |
| ASP | CD28δ | BTNL3 | CD3δ |
| ASP | CD28δ | BTNL3 | CD3γ |
| ASP | CD28δ | BTNL3 | CD3ε |
| ASP | CD28δ | BTNL3 | FcγRI-γ |
| ASP | CD28δ | BTNL3 | FcγRIII-γ |
| ASP | CD28δ | BTNL3 | FcεRIβ |
| ASP | CD28δ | BTNL3 | FcεRIγ |
| ASP | CD28δ | BTNL3 | DAP10 |
| ASP | CD28δ | BTNL3 | DAP12 |
| ASP | CD28δ | BTNL3 | CD32 |
| ASP | CD28δ | BTNL3 | CD79a |
| ASP | CD28δ | BTNL3 | CD79b |
| ASP | CD28δ | NKG2D | CD8 |
| ASP | CD28δ | NKG2D | CD3ζ |
| ASP | CD28δ | NKG2D | CD3δ |
| ASP | CD28δ | NKG2D | CD3γ |
| ASP | CD28δ | NKG2D | CD3ε |
| ASP | CD28δ | NKG2D | FcγRI-γ |
| ASP | CD28δ | NKG2D | FcγRIII-γ |
| ASP | CD28δ | NKG2D | FcεRIβ |
| ASP | CD28δ | NKG2D | FcεRIγ |
| ASP | CD28δ | NKG2D | DAP10 |
| ASP | CD28δ | NKG2D | DAP12 |
| ASP | CD28δ | NKG2D | CD32 |
| ASP | CD28δ | NKG2D | CD79a |
| ASP | CD28δ | NKG2D | CD79b |
| ASP | CD80 | CD28 | CD8 |
| ASP | CD80 | CD28 | CD3ζ |
| ASP | CD80 | CD28 | CD3δ |
| ASP | CD80 | CD28 | CD3γ |
| ASP | CD80 | CD28 | CD3ε |
| ASP | CD80 | CD28 | FcγRI-γ |
| ASP | CD80 | CD28 | FcγRIII-γ |
| ASP | CD80 | CD28 | FcεRIβ |
| ASP | CD80 | CD28 | FcεRIγ |
| ASP | CD80 | CD28 | DAP10 |
| ASP | CD80 | CD28 | DAP12 |
| ASP | CD80 | CD28 | CD32 |
| ASP | CD80 | CD28 | CD79a |
| ASP | CD80 | CD28 | CD79b |
| ASP | CD80 | CD8 | CD8 |
| ASP | CD80 | CD8 | CD3ζ |
| ASP | CD80 | CD8 | CD3δ |
| ASP | CD80 | CD8 | CD3γ |
| ASP | CD80 | CD8 | CD3ε |
| ASP | CD80 | CD8 | FcγRI-γ |
| ASP | CD80 | CD8 | FcγRIII-γ |
| ASP | CD80 | CD8 | FcεRIβ |
| ASP | CD80 | CD8 | FcεRIγ |
| ASP | CD80 | CD8 | DAP10 |
| ASP | CD80 | CD8 | DAP12 |
| ASP | CD80 | CD8 | CD32 |
| ASP | CD80 | CD8 | CD79a |
| ASP | CD80 | CD8 | CD79b |
| ASP | CD80 | CD4 | CD8 |
| ASP | CD80 | CD4 | CD3ζ |
| ASP | CD80 | CD4 | CD3δ |
| ASP | CD80 | CD4 | CD3γ |
| ASP | CD80 | CD4 | CD3ε |
| ASP | CD80 | CD4 | FcγRI-γ |
| ASP | CD80 | CD4 | FcγRIII-γ |
| ASP | CD80 | CD4 | FcεRIβ |
| ASP | CD80 | CD4 | FcεRIγ |
| ASP | CD80 | CD4 | DAP10 |
| ASP | CD80 | CD4 | DAP12 |
| ASP | CD80 | CD4 | CD32 |
| ASP | CD80 | CD4 | CD79a |
| ASP | CD80 | CD4 | CD79b |
| ASP | CD80 | b2c | CD8 |
| ASP | CD80 | b2c | CD3ζ |
| ASP | CD80 | b2c | CD3δ |
| ASP | CD80 | b2c | CD3γ |
| ASP | CD80 | b2c | CD3ε |
| ASP | CD80 | b2c | FcγRI-γ |
| ASP | CD80 | b2c | FcγRIII-γ |
| ASP | CD80 | b2c | FcεRIβ |
| ASP | CD80 | b2c | FcεRIγ |
| ASP | CD80 | b2c | DAP10 |
| ASP | CD80 | b2c | DAP12 |
| ASP | CD80 | b2c | CD32 |
| ASP | CD80 | b2c | CD79a |
| ASP | CD80 | b2c | CD79b |
| ASP | CD80 | CD137/41BB | CD8 |
| ASP | CD80 | CD137/41BB | CD3ζ |
| ASP | CD80 | CD137/41BB | CD3δ |
| ASP | CD80 | CD137/41BB | CD3γ |
| ASP | CD80 | CD137/41BB | CD3ε |
| ASP | CD80 | CD137/41BB | FcγRI-γ |
| ASP | CD80 | CD137/41BB | FcγRIII-γ |
| ASP | CD80 | CD137/41BB | FcεRIβ |
| ASP | CD80 | CD137/41BB | FcεRIγ |
| ASP | CD80 | CD137/41BB | DAP10 |
| ASP | CD80 | CD137/41BB | DAP12 |
| ASP | CD80 | CD137/41BB | CD32 |
| ASP | CD80 | CD137/41BB | CD79a |
| ASP | CD80 | CD137/41BB | CD79b |
| ASP | CD80 | ICOS | CD8 |
| ASP | CD80 | ICOS | CD3ζ |
| ASP | CD80 | ICOS | CD3δ |
| ASP | CD80 | ICOS | CD3γ |
| ASP | CD80 | ICOS | CD3ε |
| ASP | CD80 | ICOS | FcγRI-γ |
| ASP | CD80 | ICOS | FcγRIII-γ |
| ASP | CD80 | ICOS | FcεRIβ |
| ASP | CD80 | ICOS | FcεRIγ |
| ASP | CD80 | ICOS | DAP10 |
| ASP | CD80 | ICOS | DAP12 |
| ASP | CD80 | ICOS | CD32 |
| ASP | CD80 | ICOS | CD79a |
| ASP | CD80 | ICOS | CD79b |
| ASP | CD80 | CD27 | CD8 |
| ASP | CD80 | CD27 | CD3ζ |
| ASP | CD80 | CD27 | CD3δ |
| ASP | CD80 | CD27 | CD3γ |
| ASP | CD80 | CD27 | CD3ε |
| ASP | CD80 | CD27 | FcγRI-γ |
| ASP | CD80 | CD27 | FcγRIII-γ |
| ASP | CD80 | CD27 | FcεRIβ |
| ASP | CD80 | CD27 | FcεRIγ |
| ASP | CD80 | CD27 | DAP10 |
| ASP | CD80 | CD27 | DAP12 |
| ASP | CD80 | CD27 | CD32 |
| ASP | CD80 | CD27 | CD79a |
| ASP | CD80 | CD27 | CD79b |
| ASP | CD80 | CD28δ | CD8 |
| ASP | CD80 | CD28δ | CD3ζ |
| ASP | CD80 | CD28δ | CD3δ |
| ASP | CD80 | CD28δ | CD3γ |
| ASP | CD80 | CD28δ | CD3ε |
| ASP | CD80 | CD28δ | FcγRI-γ |
| ASP | CD80 | CD28δ | FcγRIII-γ |
| ASP | CD80 | CD28δ | FcεRIβ |
| ASP | CD80 | CD28δ | FcεRIγ |
| ASP | CD80 | CD28δ | DAP10 |
| ASP | CD80 | CD28δ | DAP12 |
| ASP | CD80 | CD28δ | CD32 |
| ASP | CD80 | CD28δ | CD79a |
| ASP | CD80 | CD28δ | CD79b |
| ASP | CD80 | CD80 | CD8 |
| ASP | CD80 | CD80 | CD3ζ |
| ASP | CD80 | CD80 | CD3δ |
| ASP | CD80 | CD80 | CD3γ |
| ASP | CD80 | CD80 | CD3ε |
| ASP | CD80 | CD80 | FcγRI-γ |
| ASP | CD80 | CD80 | FcγRIII-γ |
| ASP | CD80 | CD80 | FcεRIβ |
| ASP | CD80 | CD80 | FcεRIγ |
| ASP | CD80 | CD80 | DAP10 |
| ASP | CD80 | CD80 | DAP12 |
| ASP | CD80 | CD80 | CD32 |
| ASP | CD80 | CD80 | CD79a |
| ASP | CD80 | CD80 | CD79b |
| ASP | CD80 | CD86 | CD8 |
| ASP | CD80 | CD86 | CD3ζ |
| ASP | CD80 | CD86 | CD3δ |
| ASP | CD80 | CD86 | CD3γ |
| ASP | CD80 | CD86 | CD3ε |
| ASP | CD80 | CD86 | FcγRI-γ |
| ASP | CD80 | CD86 | FcγRIII-γ |
| ASP | CD80 | CD86 | FcεRIβ |
| ASP | CD80 | CD86 | FcεRIγ |
| ASP | CD80 | CD86 | DAP10 |
| ASP | CD80 | CD86 | DAP12 |
| ASP | CD80 | CD86 | CD32 |
| ASP | CD80 | CD86 | CD79a |
| ASP | CD80 | CD86 | CD79b |
| ASP | CD80 | OX40 | CD8 |
| ASP | CD80 | OX40 | CD3ζ |
| ASP | CD80 | OX40 | CD3δ |
| ASP | CD80 | OX40 | CD3γ |
| ASP | CD80 | OX40 | CD3ε |
| ASP | CD80 | OX40 | FcγRI-γ |
| ASP | CD80 | OX40 | FcγRIII-γ |
| ASP | CD80 | OX40 | FcεRIβ |
| ASP | CD80 | OX40 | FcεRIγ |
| ASP | CD80 | OX40 | DAP10 |
| ASP | CD80 | OX40 | DAP12 |
| ASP | CD80 | OX40 | CD32 |
| ASP | CD80 | OX40 | CD79a |
| ASP | CD80 | OX40 | CD79b |
| ASP | CD80 | DAP10 | CD8 |
| ASP | CD80 | DAP10 | CD3ζ |
| ASP | CD80 | DAP10 | CD3δ |
| ASP | CD80 | DAP10 | CD3γ |
| ASP | CD80 | DAP10 | CD3ε |
| ASP | CD80 | DAP10 | FcγRI-γ |
| ASP | CD80 | DAP10 | FcγRIII-γ |
| ASP | CD80 | DAP10 | FcεRIβ |
| ASP | CD80 | DAP10 | FcεRIγ |
| ASP | CD80 | DAP10 | DAP10 |
| ASP | CD80 | DAP10 | DAP12 |
| ASP | CD80 | DAP10 | CD32 |
| ASP | CD80 | DAP10 | CD79a |
| ASP | CD80 | DAP10 | CD79b |
| ASP | CD80 | DAP12 | CD8 |
| ASP | CD80 | DAP12 | CD3ζ |
| ASP | CD80 | DAP12 | CD3δ |
| ASP | CD80 | DAP12 | CD3γ |
| ASP | CD80 | DAP12 | CD3ε |
| ASP | CD80 | DAP12 | FcγRI-γ |
| ASP | CD80 | DAP12 | FcγRIII-γ |
| ASP | CD80 | DAP12 | FcεRIβ |
| ASP | CD80 | DAP12 | FcεRIγ |
| ASP | CD80 | DAP12 | DAP10 |
| ASP | CD80 | DAP12 | DAP12 |
| ASP | CD80 | DAP12 | CD32 |
| ASP | CD80 | DAP12 | CD79a |
| ASP | CD80 | DAP12 | CD79b |
| ASP | CD80 | MyD88 | CD8 |
| ASP | CD80 | MyD88 | CD3ζ |
| ASP | CD80 | MyD88 | CD3δ |
| ASP | CD80 | MyD88 | CD3γ |
| ASP | CD80 | MyD88 | CD3ε |
| ASP | CD80 | MyD88 | FcγRI-γ |
| ASP | CD80 | MyD88 | FcγRIII-γ |
| ASP | CD80 | MyD88 | FcεRIβ |
| ASP | CD80 | MyD88 | FcεRIγ |
| ASP | CD80 | MyD88 | DAP10 |
| ASP | CD80 | MyD88 | DAP12 |
| ASP | CD80 | MyD88 | CD32 |
| ASP | CD80 | MyD88 | CD79a |
| ASP | CD80 | MyD88 | CD79b |
| ASP | CD80 | CD7 | CD8 |
| ASP | CD80 | CD7 | CD3ζ |
| ASP | CD80 | CD7 | CD3δ |
| ASP | CD80 | CD7 | CD3γ |
| ASP | CD80 | CD7 | CD3ε |
| ASP | CD80 | CD7 | FcγRI-γ |
| ASP | CD80 | CD7 | FcγRIII-γ |
| ASP | CD80 | CD7 | FcεRIβ |
| ASP | CD80 | CD7 | FcεRIγ |
| ASP | CD80 | CD7 | DAP10 |
| ASP | CD80 | CD7 | DAP12 |
| ASP | CD80 | CD7 | CD32 |
| ASP | CD80 | CD7 | CD79a |
| ASP | CD80 | CD7 | CD79b |
| ASP | CD80 | BTNL3 | CD8 |
| ASP | CD80 | BTNL3 | CD3ζ |
| ASP | CD80 | BTNL3 | CD3δ |
| ASP | CD80 | BTNL3 | CD3γ |
| ASP | CD80 | BTNL3 | CD3ε |
| ASP | CD80 | BTNL3 | FcγRI-γ |
| ASP | CD80 | BTNL3 | FcγRIII-γ |
| ASP | CD80 | BTNL3 | FcεRIβ |
| ASP | CD80 | BTNL3 | FcεRIγ |
| ASP | CD80 | BTNL3 | DAP10 |
| ASP | CD80 | BTNL3 | DAP12 |
| ASP | CD80 | BTNL3 | CD32 |
| ASP | CD80 | BTNL3 | CD79a |
| ASP | CD80 | BTNL3 | CD79b |
| ASP | CD80 | NKG2D | CD8 |
| ASP | CD80 | NKG2D | CD3ζ |
| ASP | CD80 | NKG2D | CD3δ |
| ASP | CD80 | NKG2D | CD3γ |
| ASP | CD80 | NKG2D | CD3ε |
| ASP | CD80 | NKG2D | FcγRI-γ |
| ASP | CD80 | NKG2D | FcγRIII-γ |
| ASP | CD80 | NKG2D | FcεRIβ |
| ASP | CD80 | NKG2D | FcεRIγ |
| ASP | CD80 | NKG2D | DAP10 |
| ASP | CD80 | NKG2D | DAP12 |
| ASP | CD80 | NKG2D | CD32 |
| ASP | CD80 | NKG2D | CD79a |
| ASP | CD80 | NKG2D | CD79b |
| ASP | CD86 | CD28 | CD8 |
| ASP | CD86 | CD28 | CD3ζ |
| ASP | CD86 | CD28 | CD3δ |
| ASP | CD86 | CD28 | CD3γ |
| ASP | CD86 | CD28 | CD3ε |
| ASP | CD86 | CD28 | FcγRI-γ |
| ASP | CD86 | CD28 | FcγRIII-γ |
| ASP | CD86 | CD28 | FcεRIβ |
| ASP | CD86 | CD28 | FcεRIγ |
| ASP | CD86 | CD28 | DAP10 |
| ASP | CD86 | CD28 | DAP12 |
| ASP | CD86 | CD28 | CD32 |
| ASP | CD86 | CD28 | CD79a |
| ASP | CD86 | CD28 | CD79b |
| ASP | CD86 | CD8 | CD8 |
| ASP | CD86 | CD8 | CD3ζ |
| ASP | CD86 | CD8 | CD3δ |
| ASP | CD86 | CD8 | CD3γ |
| ASP | CD86 | CD8 | CD3ε |
| ASP | CD86 | CD8 | FcγRI-γ |
| ASP | CD86 | CD8 | FcγRIII-γ |
| ASP | CD86 | CD8 | FcεRIβ |
| ASP | CD86 | CD8 | FcεRIγ |
| ASP | CD86 | CD8 | DAP10 |
| ASP | CD86 | CD8 | DAP12 |
| ASP | CD86 | CD8 | CD32 |
| ASP | CD86 | CD8 | CD79a |
| ASP | CD86 | CD8 | CD79b |
| ASP | CD86 | CD4 | CD8 |
| ASP | CD86 | CD4 | CD3ζ |
| ASP | CD86 | CD4 | CD3δ |
| ASP | CD86 | CD4 | CD3γ |
| ASP | CD86 | CD4 | CD3ε |
| ASP | CD86 | CD4 | FcγRI-γ |
| ASP | CD86 | CD4 | FcγRIII-γ |
| ASP | CD86 | CD4 | FcεRIβ |
| ASP | CD86 | CD4 | FcεRIγ |
| ASP | CD86 | CD4 | DAP10 |
| ASP | CD86 | CD4 | DAP12 |
| ASP | CD86 | CD4 | CD32 |
| ASP | CD86 | CD4 | CD79a |
| ASP | CD86 | CD4 | CD79b |
| ASP | CD86 | b2c | CD8 |
| ASP | CD86 | b2c | CD3ζ |
| ASP | CD86 | b2c | CD3δ |
| ASP | CD86 | b2c | CD3γ |
| ASP | CD86 | b2c | CD3ε |
| ASP | CD86 | b2c | FcγRI-γ |
| ASP | CD86 | b2c | FcγRIII-γ |
| ASP | CD86 | b2c | FcεRIβ |
| ASP | CD86 | b2c | FcεRIγ |
| ASP | CD86 | b2c | DAP10 |
| ASP | CD86 | b2c | DAP12 |
| ASP | CD86 | b2c | CD32 |
| ASP | CD86 | b2c | CD79a |
| ASP | CD86 | b2c | CD79b |
| ASP | CD86 | CD137/41BB | CD8 |
| ASP | CD86 | CD137/41BB | CD3ζ |
| ASP | CD86 | CD137/41BB | CD3δ |
| ASP | CD86 | CD137/41BB | CD3γ |
| ASP | CD86 | CD137/41BB | CD3ε |
| ASP | CD86 | CD137/41BB | FcγRI-γ |
| ASP | CD86 | CD137/41BB | FcγRIII-γ |
| ASP | CD86 | CD137/41BB | FcεRIβ |
| ASP | CD86 | CD137/41BB | FcεRIγ |
| ASP | CD86 | CD137/41BB | DAP10 |
| ASP | CD86 | CD137/41BB | DAP12 |
| ASP | CD86 | CD137/41BB | CD32 |
| ASP | CD86 | CD137/41BB | CD79a |
| ASP | CD86 | CD137/41BB | CD79b |
| ASP | CD86 | ICOS | CD8 |
| ASP | CD86 | ICOS | CD3ζ |
| ASP | CD86 | ICOS | CD3δ |
| ASP | CD86 | ICOS | CD3γ |
| ASP | CD86 | ICOS | CD3ε |
| ASP | CD86 | ICOS | FcγRI-γ |
| ASP | CD86 | ICOS | FcγRIII-γ |
| ASP | CD86 | ICOS | FcεRIβ |
| ASP | CD86 | ICOS | FcεRIγ |
| ASP | CD86 | ICOS | DAP10 |
| ASP | CD86 | ICOS | DAP12 |
| ASP | CD86 | ICOS | CD32 |
| ASP | CD86 | ICOS | CD79a |
| ASP | CD86 | ICOS | CD79b |
| ASP | CD86 | CD27 | CD8 |
| ASP | CD86 | CD27 | CD3ζ |
| ASP | CD86 | CD27 | CD3δ |
| ASP | CD86 | CD27 | CD3γ |
| ASP | CD86 | CD27 | CD3ε |
| ASP | CD86 | CD27 | FcγRI-γ |
| ASP | CD86 | CD27 | FcγRIII-γ |
| ASP | CD86 | CD27 | FcεRIβ |
| ASP | CD86 | CD27 | FcεRIγ |
| ASP | CD86 | CD27 | DAP10 |
| ASP | CD86 | CD27 | DAP12 |
| ASP | CD86 | CD27 | CD32 |
| ASP | CD86 | CD27 | CD79a |
| ASP | CD86 | CD27 | CD79b |
| ASP | CD86 | CD28δ | CD8 |
| ASP | CD86 | CD28δ | CD3ζ |
| ASP | CD86 | CD28δ | CD3δ |
| ASP | CD86 | CD28δ | CD3γ |
| ASP | CD86 | CD28δ | CD3ε |
| ASP | CD86 | CD28δ | FcγRI-γ |
| ASP | CD86 | CD28δ | FcγRIII-γ |
| ASP | CD86 | CD28δ | FcεRIβ |
| ASP | CD86 | CD28δ | FcεRIγ |
| ASP | CD86 | CD28δ | DAP10 |
| ASP | CD86 | CD28δ | DAP12 |
| ASP | CD86 | CD28δ | CD32 |
| ASP | CD86 | CD28δ | CD79a |
| ASP | CD86 | CD28δ | CD79b |
| ASP | CD86 | CD80 | CD8 |
| ASP | CD86 | CD80 | CD3ζ |
| ASP | CD86 | CD80 | CD3δ |
| ASP | CD86 | CD80 | CD3γ |
| ASP | CD86 | CD80 | CD3ε |
| ASP | CD86 | CD80 | FcγRI-γ |
| ASP | CD86 | CD80 | FcγRIII-γ |
| ASP | CD86 | CD80 | FcεRIβ |
| ASP | CD86 | CD80 | FcεRIγ |
| ASP | CD86 | CD80 | DAP10 |
| ASP | CD86 | CD80 | DAP12 |
| ASP | CD86 | CD80 | CD32 |
| ASP | CD86 | CD80 | CD79a |
| ASP | CD86 | CD80 | CD79b |
| ASP | CD86 | CD86 | CD8 |
| ASP | CD86 | CD86 | CD3ζ |
| ASP | CD86 | CD86 | CD3δ |
| ASP | CD86 | CD86 | CD3γ |
| ASP | CD86 | CD86 | CD3ε |
| ASP | CD86 | CD86 | FcγRI-γ |
| ASP | CD86 | CD86 | FcγRIII-γ |
| ASP | CD86 | CD86 | FcεRIβ |
| ASP | CD86 | CD86 | FcεRIγ |
| ASP | CD86 | CD86 | DAP10 |
| ASP | CD86 | CD86 | DAP12 |
| ASP | CD86 | CD86 | CD32 |
| ASP | CD86 | CD86 | CD79a |
| ASP | CD86 | CD86 | CD79b |
| ASP | CD86 | OX40 | CD8 |
| ASP | CD86 | OX40 | CD3ζ |
| ASP | CD86 | OX40 | CD3δ |
| ASP | CD86 | OX40 | CD3γ |
| ASP | CD86 | OX40 | CD3ε |
| ASP | CD86 | OX40 | FcγRI-γ |
| ASP | CD86 | OX40 | FcγRIII-γ |
| ASP | CD86 | OX40 | FcεRIβ |
| ASP | CD86 | OX40 | FcεRIγ |
| ASP | CD86 | OX40 | DAP10 |
| ASP | CD86 | OX40 | DAP12 |
| ASP | CD86 | OX40 | CD32 |
| ASP | CD86 | OX40 | CD79a |
| ASP | CD86 | OX40 | CD79b |
| ASP | CD86 | DAP10 | CD8 |
| ASP | CD86 | DAP10 | CD3ζ |
| ASP | CD86 | DAP10 | CD3δ |
| ASP | CD86 | DAP10 | CD3γ |
| ASP | CD86 | DAP10 | CD3ε |
| ASP | CD86 | DAP10 | FcγRI-γ |
| ASP | CD86 | DAP10 | FcγRIII-γ |
| ASP | CD86 | DAP10 | FcεRIβ |
| ASP | CD86 | DAP10 | FcεRIγ |
| ASP | CD86 | DAP10 | DAP10 |
| ASP | CD86 | DAP10 | DAP12 |
| ASP | CD86 | DAP10 | CD32 |
| ASP | CD86 | DAP10 | CD79a |
| ASP | CD86 | DAP10 | CD79b |
| ASP | CD86 | DAP12 | CD8 |
| ASP | CD86 | DAP12 | CD3ζ |
| ASP | CD86 | DAP12 | CD3δ |
| ASP | CD86 | DAP12 | CD3γ |
| ASP | CD86 | DAP12 | CD3ε |
| ASP | CD86 | DAP12 | FcγRI-γ |
| ASP | CD86 | DAP12 | FcγRIII-γ |
| ASP | CD86 | DAP12 | FcεRIβ |
| ASP | CD86 | DAP12 | FcεRIγ |
| ASP | CD86 | DAP12 | DAP10 |
| ASP | CD86 | DAP12 | DAP12 |
| ASP | CD86 | DAP12 | CD32 |
| ASP | CD86 | DAP12 | CD79a |
| ASP | CD86 | DAP12 | CD79b |
| ASP | CD86 | MyD88 | CD8 |
| ASP | CD86 | MyD88 | CD3ζ |
| ASP | CD86 | MyD88 | CD3δ |
| ASP | CD86 | MyD88 | CD3γ |
| ASP | CD86 | MyD88 | CD3ε |
| ASP | CD86 | MyD88 | FcγRI-γ |
| ASP | CD86 | MyD88 | FcγRIII-γ |
| ASP | CD86 | MyD88 | FcεRIβ |
| ASP | CD86 | MyD88 | FcεRIγ |
| ASP | CD86 | MyD88 | DAP10 |
| ASP | CD86 | MyD88 | DAP12 |
| ASP | CD86 | MyD88 | CD32 |
| ASP | CD86 | MyD88 | CD79a |
| ASP | CD86 | MyD88 | CD79b |
| ASP | CD86 | CD7 | CD8 |
| ASP | CD86 | CD7 | CD3ζ |
| ASP | CD86 | CD7 | CD3δ |
| ASP | CD86 | CD7 | CD3γ |
| ASP | CD86 | CD7 | CD3ε |
| ASP | CD86 | CD7 | FcγRI-γ |
| ASP | CD86 | CD7 | FcγRIII-γ |
| ASP | CD86 | CD7 | FcεRIβ |
| ASP | CD86 | CD7 | FcεRIγ |
| ASP | CD86 | CD7 | DAP10 |
| ASP | CD86 | CD7 | DAP12 |
| ASP | CD86 | CD7 | CD32 |
| ASP | CD86 | CD7 | CD79a |
| ASP | CD86 | CD7 | CD79b |
| ASP | CD86 | BTNL3 | CD8 |
| ASP | CD86 | BTNL3 | CD3ζ |
| ASP | CD86 | BTNL3 | CD3δ |
| ASP | CD86 | BTNL3 | CD3γ |
| ASP | CD86 | BTNL3 | CD3ε |
| ASP | CD86 | BTNL3 | FcγRI-γ |
| ASP | CD86 | BTNL3 | FcγRIII-γ |
| ASP | CD86 | BTNL3 | FcεRIβ |
| ASP | CD86 | BTNL3 | FcεRIγ |
| ASP | CD86 | BTNL3 | DAP10 |
| ASP | CD86 | BTNL3 | DAP12 |
| ASP | CD86 | BTNL3 | CD32 |
| ASP | CD86 | BTNL3 | CD79a |
| ASP | CD86 | BTNL3 | CD79b |
| ASP | CD86 | NKG2D | CD8 |
| ASP | CD86 | NKG2D | CD3ζ |
| ASP | CD86 | NKG2D | CD3δ |
| ASP | CD86 | NKG2D | CD3γ |
| ASP | CD86 | NKG2D | CD3ε |
| ASP | CD86 | NKG2D | FcγRI-γ |
| ASP | CD86 | NKG2D | FcγRIII-γ |
| ASP | CD86 | NKG2D | FcεRIβ |
| ASP | CD86 | NKG2D | FcεRIγ |
| ASP | CD86 | NKG2D | DAP10 |
| ASP | CD86 | NKG2D | DAP12 |
| ASP | CD86 | NKG2D | CD32 |
| ASP | CD86 | NKG2D | CD79a |
| ASP | CD86 | NKG2D | CD79b |
| ASP | OX40 | CD28 | CD8 |
| ASP | OX40 | CD28 | CD3ζ |
| ASP | OX40 | CD28 | CD3δ |
| ASP | OX40 | CD28 | CD3γ |
| ASP | OX40 | CD28 | CD3ε |
| ASP | OX40 | CD28 | FcγRI-γ |
| ASP | OX40 | CD28 | FcγRIII-γ |
| ASP | OX40 | CD28 | FcεRIβ |
| ASP | OX40 | CD28 | FcεRIγ |
| ASP | OX40 | CD28 | DAP10 |
| ASP | OX40 | CD28 | DAP12 |
| ASP | OX40 | CD28 | CD32 |
| ASP | OX40 | CD28 | CD79a |
| ASP | OX40 | CD28 | CD79b |
| ASP | OX40 | CD8 | CD8 |
| ASP | OX40 | CD8 | CD3ζ |
| ASP | OX40 | CD8 | CD3δ |
| ASP | OX40 | CD8 | CD3γ |
| ASP | OX40 | CD8 | CD3ε |
| ASP | OX40 | CD8 | FcγRI-γ |
| ASP | OX40 | CD8 | FcγRIII-γ |
| ASP | OX40 | CD8 | FcεRIβ |
| ASP | OX40 | CD8 | FcεRIγ |
| ASP | OX40 | CD8 | DAP10 |
| ASP | OX40 | CD8 | DAP12 |
| ASP | OX40 | CD8 | CD32 |
| ASP | OX40 | CD8 | CD79a |
| ASP | OX40 | CD8 | CD79b |
| ASP | OX40 | CD4 | CD8 |
| ASP | OX40 | CD4 | CD3ζ |
| ASP | OX40 | CD4 | CD3δ |
| ASP | OX40 | CD4 | CD3γ |
| ASP | OX40 | CD4 | CD3ε |
| ASP | OX40 | CD4 | FcγRI-γ |
| ASP | OX40 | CD4 | FcγRIII-γ |
| ASP | OX40 | CD4 | FcεRIβ |
| ASP | OX40 | CD4 | FcεRIγ |
| ASP | OX40 | CD4 | DAP10 |
| ASP | OX40 | CD4 | DAP12 |
| ASP | OX40 | CD4 | CD32 |
| ASP | OX40 | CD4 | CD79a |
| ASP | OX40 | CD4 | CD79b |
| ASP | OX40 | b2c | CD8 |
| ASP | OX40 | b2c | CD3ζ |
| ASP | OX40 | b2c | CD3δ |
| ASP | OX40 | b2c | CD3γ |
| ASP | OX40 | b2c | CD3ε |
| ASP | OX40 | b2c | FcγRI-γ |
| ASP | OX40 | b2c | FcγRIII-γ |
| ASP | OX40 | b2c | FcεRIβ |
| ASP | OX40 | b2c | FcεRIγ |
| ASP | OX40 | b2c | DAP10 |
| ASP | OX40 | b2c | DAP12 |
| ASP | OX40 | b2c | CD32 |
| ASP | OX40 | b2c | CD79a |
| ASP | OX40 | b2c | CD79b |
| ASP | OX40 | CD137/41BB | CD8 |
| ASP | OX40 | CD137/41BB | CD3ζ |
| ASP | OX40 | CD137/41BB | CD3δ |
| ASP | OX40 | CD137/41BB | CD3γ |
| ASP | OX40 | CD137/41BB | CD3ε |
| ASP | OX40 | CD137/41BB | FcγRI-γ |
| ASP | OX40 | CD137/41BB | FcγRIII-γ |
| ASP | OX40 | CD137/41BB | FcεRIβ |
| ASP | OX40 | CD137/41BB | FcεRIγ |
| ASP | OX40 | CD137/41BB | DAP10 |
| ASP | OX40 | CD137/41BB | DAP12 |
| ASP | OX40 | CD137/41BB | CD32 |
| ASP | OX40 | CD137/41BB | CD79a |
| ASP | OX40 | CD137/41BB | CD79b |
| ASP | OX40 | ICOS | CD8 |
| ASP | OX40 | ICOS | CD3ζ |
| ASP | OX40 | ICOS | CD3δ |
| ASP | OX40 | ICOS | CD3γ |
| ASP | OX40 | ICOS | CD3ε |
| ASP | OX40 | ICOS | FcγRI-γ |
| ASP | OX40 | ICOS | FcγRIII-γ |
| ASP | OX40 | ICOS | FcεRIβ |
| ASP | OX40 | ICOS | FcεRIγ |
| ASP | OX40 | ICOS | DAP10 |
| ASP | OX40 | ICOS | DAP12 |
| ASP | OX40 | ICOS | CD32 |
| ASP | OX40 | ICOS | CD79a |
| ASP | OX40 | ICOS | CD79b |
| ASP | OX40 | CD27 | CD8 |
| ASP | OX40 | CD27 | CD3ζ |
| ASP | OX40 | CD27 | CD3δ |
| ASP | OX40 | CD27 | CD3γ |
| ASP | OX40 | CD27 | CD3ε |
| ASP | OX40 | CD27 | FcγRI-γ |
| ASP | OX40 | CD27 | FcγRIII-γ |
| ASP | OX40 | CD27 | FcεRIβ |
| ASP | OX40 | CD27 | FcεRIγ |
| ASP | OX40 | CD27 | DAP10 |
| ASP | OX40 | CD27 | DAP12 |
| ASP | OX40 | CD27 | CD32 |
| ASP | OX40 | CD27 | CD79a |
| ASP | OX40 | CD27 | CD79b |
| ASP | OX40 | CD28δ | CD8 |
| ASP | OX40 | CD28δ | CD3ζ |
| ASP | OX40 | CD28δ | CD3δ |
| ASP | OX40 | CD28δ | CD3γ |
| ASP | OX40 | CD28δ | CD3ε |
| ASP | OX40 | CD28δ | FcγRI-γ |
| ASP | OX40 | CD28δ | FcγRIII-γ |
| ASP | OX40 | CD28δ | FcεRIβ |
| ASP | OX40 | CD28δ | FcεRIγ |
| ASP | OX40 | CD28δ | DAP10 |
| ASP | OX40 | CD28δ | DAP12 |
| ASP | OX40 | CD28δ | CD32 |
| ASP | OX40 | CD28δ | CD79a |
| ASP | OX40 | CD28δ | CD79b |
| ASP | OX40 | CD80 | CD8 |
| ASP | OX40 | CD80 | CD3ζ |
| ASP | OX40 | CD80 | CD3δ |
| ASP | OX40 | CD80 | CD3γ |
| ASP | OX40 | CD80 | CD3ε |
| ASP | OX40 | CD80 | FcγRI-γ |
| ASP | OX40 | CD80 | FcγRIII-γ |
| ASP | OX40 | CD80 | FcεRIβ |
| ASP | OX40 | CD80 | FcεRIγ |
| ASP | OX40 | CD80 | DAP10 |
| ASP | OX40 | CD80 | DAP12 |
| ASP | OX40 | CD80 | CD32 |
| ASP | OX40 | CD80 | CD79a |
| ASP | OX40 | CD80 | CD79b |
| ASP | OX40 | CD86 | CD8 |
| ASP | OX40 | CD86 | CD3ζ |
| ASP | OX40 | CD86 | CD3δ |
| ASP | OX40 | CD86 | CD3γ |
| ASP | OX40 | CD86 | CD3ε |
| ASP | OX40 | CD86 | FcγRI-γ |
| ASP | OX40 | CD86 | FcγRIII-γ |
| ASP | OX40 | CD86 | FcεRIβ |
| ASP | OX40 | CD86 | FcεRIγ |
| ASP | OX40 | CD86 | DAP10 |
| ASP | OX40 | CD86 | DAP12 |
| ASP | OX40 | CD86 | CD32 |
| ASP | OX40 | CD86 | CD79a |
| ASP | OX40 | CD86 | CD79b |
| ASP | OX40 | OX40 | CD8 |
| ASP | OX40 | OX40 | CD3ζ |
| ASP | OX40 | OX40 | CD3δ |
| ASP | OX40 | OX40 | CD3γ |
| ASP | OX40 | OX40 | CD3ε |
| ASP | OX40 | OX40 | FcγRI-γ |
| ASP | OX40 | OX40 | FcγRIII-γ |
| ASP | OX40 | OX40 | FcεRIβ |
| ASP | OX40 | OX40 | FcεRIγ |
| ASP | OX40 | OX40 | DAP10 |
| ASP | OX40 | OX40 | DAP12 |
| ASP | OX40 | OX40 | CD32 |
| ASP | OX40 | OX40 | CD79a |
| ASP | OX40 | OX40 | CD79b |
| ASP | OX40 | DAP10 | CD8 |
| ASP | OX40 | DAP10 | CD3ζ |
| ASP | OX40 | DAP10 | CD3δ |
| ASP | OX40 | DAP10 | CD3γ |
| ASP | OX40 | DAP10 | CD3ε |
| ASP | OX40 | DAP10 | FcγRI-γ |
| ASP | OX40 | DAP10 | FcγRIII-γ |
| ASP | OX40 | DAP10 | FcεRIβ |
| ASP | OX40 | DAP10 | FcεRIγ |
| ASP | OX40 | DAP10 | DAP10 |
| ASP | OX40 | DAP10 | DAP12 |
| ASP | OX40 | DAP10 | CD32 |
| ASP | OX40 | DAP10 | CD79a |
| ASP | OX40 | DAP10 | CD79b |
| ASP | OX40 | DAP12 | CD8 |
| ASP | OX40 | DAP12 | CD3ζ |
| ASP | OX40 | DAP12 | CD3δ |
| ASP | OX40 | DAP12 | CD3γ |
| ASP | OX40 | DAP12 | CD3ε |
| ASP | OX40 | DAP12 | FcγRI-γ |
| ASP | OX40 | DAP12 | FcγRIII-γ |
| ASP | OX40 | DAP12 | FcεRIβ |
| ASP | OX40 | DAP12 | FcεRIγ |
| ASP | OX40 | DAP12 | DAP10 |
| ASP | OX40 | DAP12 | DAP12 |
| ASP | OX40 | DAP12 | CD32 |
| ASP | OX40 | DAP12 | CD79a |
| ASP | OX40 | DAP12 | CD79b |
| ASP | OX40 | MyD88 | CD8 |
| ASP | OX40 | MyD88 | CD3ζ |
| ASP | OX40 | MyD88 | CD3δ |
| ASP | OX40 | MyD88 | CD3γ |
| ASP | OX40 | MyD88 | CD3ε |
| ASP | OX40 | MyD88 | FcγRI-γ |
| ASP | OX40 | MyD88 | FcγRIII-γ |
| ASP | OX40 | MyD88 | FcεRIβ |
| ASP | OX40 | MyD88 | FcεRIγ |
| ASP | OX40 | MyD88 | DAP10 |
| ASP | OX40 | MyD88 | DAP12 |
| ASP | OX40 | MyD88 | CD32 |
| ASP | OX40 | MyD88 | CD79a |
| ASP | OX40 | MyD88 | CD79b |
| ASP | OX40 | CD7 | CD8 |
| ASP | OX40 | CD7 | CD3ζ |
| ASP | OX40 | CD7 | CD3δ |
| ASP | OX40 | CD7 | CD3γ |
| ASP | OX40 | CD7 | CD3ε |
| ASP | OX40 | CD7 | FcγRI-γ |
| ASP | OX40 | CD7 | FcγRIII-γ |
| ASP | OX40 | CD7 | FcεRIβ |
| ASP | OX40 | CD7 | FcεRIγ |
| ASP | OX40 | CD7 | DAP10 |
| ASP | OX40 | CD7 | DAP12 |
| ASP | OX40 | CD7 | CD32 |
| ASP | OX40 | CD7 | CD79a |
| ASP | OX40 | CD7 | CD79b |
| ASP | OX40 | BTNL3 | CD8 |
| ASP | OX40 | BTNL3 | CD3ζ |
| ASP | OX40 | BTNL3 | CD3δ |
| ASP | OX40 | BTNL3 | CD3γ |
| ASP | OX40 | BTNL3 | CD3ε |
| ASP | OX40 | BTNL3 | FcγRI-γ |
| ASP | OX40 | BTNL3 | FcγRIII-γ |
| ASP | OX40 | BTNL3 | FcεRIβ |
| ASP | OX40 | BTNL3 | FcεRIγ |
| ASP | OX40 | BTNL3 | DAP10 |
| ASP | OX40 | BTNL3 | DAP12 |
| ASP | OX40 | BTNL3 | CD32 |
| ASP | OX40 | BTNL3 | CD79a |
| ASP | OX40 | BTNL3 | CD79b |
| ASP | OX40 | NKG2D | CD8 |
| ASP | OX40 | NKG2D | CD3ζ |
| ASP | OX40 | NKG2D | CD3δ |
| ASP | OX40 | NKG2D | CD3γ |
| ASP | OX40 | NKG2D | CD3ε |
| ASP | OX40 | NKG2D | FcγRI-γ |
| ASP | OX40 | NKG2D | FcγRIII-γ |
| ASP | OX40 | NKG2D | FcεRIβ |
| ASP | OX40 | NKG2D | FcεRIγ |
| ASP | OX40 | NKG2D | DAP10 |
| ASP | OX40 | NKG2D | DAP12 |
| ASP | OX40 | NKG2D | CD32 |
| ASP | OX40 | NKG2D | CD79a |
| ASP | OX40 | NKG2D | CD79b |
| ASP | DAP10 | CD28 | CD8 |
| ASP | DAP10 | CD28 | CD3ζ |
| ASP | DAP10 | CD28 | CD3δ |
| ASP | DAP10 | CD28 | CD3γ |
| ASP | DAP10 | CD28 | CD3ε |
| ASP | DAP10 | CD28 | FcγRI-γ |
| ASP | DAP10 | CD28 | FcγRIII-γ |
| ASP | DAP10 | CD28 | FcεRIβ |
| ASP | DAP10 | CD28 | FcεRIγ |
| ASP | DAP10 | CD28 | DAP10 |
| ASP | DAP10 | CD28 | DAP12 |
| ASP | DAP10 | CD28 | CD32 |
| ASP | DAP10 | CD28 | CD79a |
| ASP | DAP10 | CD28 | CD79b |
| ASP | DAP10 | CD8 | CD8 |
| ASP | DAP10 | CD8 | CD3ζ |
| ASP | DAP10 | CD8 | CD3δ |
| ASP | DAP10 | CD8 | CD3γ |
| ASP | DAP10 | CD8 | CD3ε |
| ASP | DAP10 | CD8 | FcγRI-γ |
| ASP | DAP10 | CD8 | FcγRIII-γ |
| ASP | DAP10 | CD8 | FcεRIβ |
| ASP | DAP10 | CD8 | FcεRIγ |
| ASP | DAP10 | CD8 | DAP10 |
| ASP | DAP10 | CD8 | DAP12 |
| ASP | DAP10 | CD8 | CD32 |
| ASP | DAP10 | CD8 | CD79a |
| ASP | DAP10 | CD8 | CD79b |
| ASP | DAP10 | CD4 | CD8 |
| ASP | DAP10 | CD4 | CD3ζ |
| ASP | DAP10 | CD4 | CD3δ |
| ASP | DAP10 | CD4 | CD3γ |
| ASP | DAP10 | CD4 | CD3ε |
| ASP | DAP10 | CD4 | FcγRI-γ |
| ASP | DAP10 | CD4 | FcγRIII-γ |
| ASP | DAP10 | CD4 | FcεRIβ |
| ASP | DAP10 | CD4 | FcεRIγ |
| ASP | DAP10 | CD4 | DAP10 |
| ASP | DAP10 | CD4 | DAP12 |
| ASP | DAP10 | CD4 | CD32 |
| ASP | DAP10 | CD4 | CD79a |
| ASP | DAP10 | CD4 | CD79b |
| ASP | DAP10 | b2c | CD8 |
| ASP | DAP10 | b2c | CD3ζ |
| ASP | DAP10 | b2c | CD3δ |
| ASP | DAP10 | b2c | CD3γ |
| ASP | DAP10 | b2c | CD3ε |
| ASP | DAP10 | b2c | FcγRI-γ |
| ASP | DAP10 | b2c | FcγRIII-γ |
| ASP | DAP10 | b2c | FcεRIβ |
| ASP | DAP10 | b2c | FcεRIγ |
| ASP | DAP10 | b2c | DAP10 |
| ASP | DAP10 | b2c | DAP12 |
| ASP | DAP10 | b2c | CD32 |
| ASP | DAP10 | b2c | CD79a |
| ASP | DAP10 | b2c | CD79b |
| ASP | DAP10 | CD137/41BB | CD8 |
| ASP | DAP10 | CD137/41BB | CD3ζ |
| ASP | DAP10 | CD137/41BB | CD3δ |
| ASP | DAP10 | CD137/41BB | CD3γ |
| ASP | DAP10 | CD137/41BB | CD3ε |
| ASP | DAP10 | CD137/41BB | FcγRI-γ |
| ASP | DAP10 | CD137/41BB | FcγRIII-γ |
| ASP | DAP10 | CD137/41BB | FcεRIβ |
| ASP | DAP10 | CD137/41BB | FcεRIγ |
| ASP | DAP10 | CD137/41BB | DAP10 |
| ASP | DAP10 | CD137/41BB | DAP12 |
| ASP | DAP10 | CD137/41BB | CD32 |
| ASP | DAP10 | CD137/41BB | CD79a |
| ASP | DAP10 | CD137/41BB | CD79b |
| ASP | DAP10 | ICOS | CD8 |
| ASP | DAP10 | ICOS | CD3ζ |
| ASP | DAP10 | ICOS | CD3δ |
| ASP | DAP10 | ICOS | CD3γ |
| ASP | DAP10 | ICOS | CD3ε |
| ASP | DAP10 | ICOS | FcγRI-γ |
| ASP | DAP10 | ICOS | FcγRIII-γ |
| ASP | DAP10 | ICOS | FcεRIβ |
| ASP | DAP10 | ICOS | FcεRIγ |
| ASP | DAP10 | ICOS | DAP10 |
| ASP | DAP10 | ICOS | DAP12 |
| ASP | DAP10 | ICOS | CD32 |
| ASP | DAP10 | ICOS | CD79a |
| ASP | DAP10 | ICOS | CD79b |
| ASP | DAP10 | CD27 | CD8 |
| ASP | DAP10 | CD27 | CD3ζ |
| ASP | DAP10 | CD27 | CD3δ |
| ASP | DAP10 | CD27 | CD3γ |
| ASP | DAP10 | CD27 | CD3ε |
| ASP | DAP10 | CD27 | FcγRI-γ |
| ASP | DAP10 | CD27 | FcγRIII-γ |
| ASP | DAP10 | CD27 | FcεRIβ |
| ASP | DAP10 | CD27 | FcεRIγ |
| ASP | DAP10 | CD27 | DAP10 |
| ASP | DAP10 | CD27 | DAP12 |
| ASP | DAP10 | CD27 | CD32 |
| ASP | DAP10 | CD27 | CD79a |
| ASP | DAP10 | CD27 | CD79b |
| ASP | DAP10 | CD28δ | CD8 |
| ASP | DAP10 | CD28δ | CD3ζ |
| ASP | DAP10 | CD28δ | CD3δ |
| ASP | DAP10 | CD28δ | CD3γ |
| ASP | DAP10 | CD28δ | CD3ε |
| ASP | DAP10 | CD28δ | FcγRI-γ |
| ASP | DAP10 | CD28δ | FcγRIII-γ |
| ASP | DAP10 | CD28δ | FcεRIβ |
| ASP | DAP10 | CD28δ | FcεRIγ |
| ASP | DAP10 | CD28δ | DAP10 |
| ASP | DAP10 | CD28δ | DAP12 |
| ASP | DAP10 | CD28δ | CD32 |
| ASP | DAP10 | CD28δ | CD79a |
| ASP | DAP10 | CD28δ | CD79b |
| ASP | DAP10 | CD80 | CD8 |
| ASP | DAP10 | CD80 | CD3ζ |
| ASP | DAP10 | CD80 | CD3δ |
| ASP | DAP10 | CD80 | CD3γ |
| ASP | DAP10 | CD80 | CD3ε |
| ASP | DAP10 | CD80 | FcγRI-γ |
| ASP | DAP10 | CD80 | FcγRIII-γ |
| ASP | DAP10 | CD80 | FcεRIβ |
| ASP | DAP10 | CD80 | FcεRIγ |
| ASP | DAP10 | CD80 | DAP10 |
| ASP | DAP10 | CD80 | DAP12 |
| ASP | DAP10 | CD80 | CD32 |
| ASP | DAP10 | CD80 | CD79a |
| ASP | DAP10 | CD80 | CD79b |
| ASP | DAP10 | CD86 | CD8 |
| ASP | DAP10 | CD86 | CD3ζ |
| ASP | DAP10 | CD86 | CD3δ |
| ASP | DAP10 | CD86 | CD3γ |
| ASP | DAP10 | CD86 | CD3ε |
| ASP | DAP10 | CD86 | FcγRI-γ |
| ASP | DAP10 | CD86 | FcγRIII-γ |
| ASP | DAP10 | CD86 | FcεRIβ |
| ASP | DAP10 | CD86 | FcεRIγ |
| ASP | DAP10 | CD86 | DAP10 |
| ASP | DAP10 | CD86 | DAP12 |
| ASP | DAP10 | CD86 | CD32 |
| ASP | DAP10 | CD86 | CD79a |
| ASP | DAP10 | CD86 | CD79b |
| ASP | DAP10 | OX40 | CD8 |
| ASP | DAP10 | OX40 | CD3ζ |
| ASP | DAP10 | OX40 | CD3δ |
| ASP | DAP10 | OX40 | CD3γ |
| ASP | DAP10 | OX40 | CD3ε |
| ASP | DAP10 | OX40 | FcγRI-γ |
| ASP | DAP10 | OX40 | FcγRIII-γ |
| ASP | DAP10 | OX40 | FcεRIβ |
| ASP | DAP10 | OX40 | FcεRIγ |
| ASP | DAP10 | OX40 | DAP10 |
| ASP | DAP10 | OX40 | DAP12 |
| ASP | DAP10 | OX40 | CD32 |
| ASP | DAP10 | OX40 | CD79a |
| ASP | DAP10 | OX40 | CD79b |
| ASP | DAP10 | DAP10 | CD8 |
| ASP | DAP10 | DAP10 | CD3ζ |
| ASP | DAP10 | DAP10 | CD3δ |
| ASP | DAP10 | DAP10 | CD3γ |
| ASP | DAP10 | DAP10 | CD3ε |
| ASP | DAP10 | DAP10 | FcγRI-γ |
| ASP | DAP10 | DAP10 | FcγRIII-γ |
| ASP | DAP10 | DAP10 | FcεRIβ |
| ASP | DAP10 | DAP10 | FcεRIγ |
| ASP | DAP10 | DAP10 | DAP10 |
| ASP | DAP10 | DAP10 | DAP12 |
| ASP | DAP10 | DAP10 | CD32 |
| ASP | DAP10 | DAP10 | CD79a |
| ASP | DAP10 | DAP10 | CD79b |
| ASP | DAP10 | DAP12 | CD8 |
| ASP | DAP10 | DAP12 | CD3ζ |
| ASP | DAP10 | DAP12 | CD3δ |
| ASP | DAP10 | DAP12 | CD3γ |
| ASP | DAP10 | DAP12 | CD3ε |
| ASP | DAP10 | DAP12 | FcγRI-γ |
| ASP | DAP10 | DAP12 | FcγRIII-γ |
| ASP | DAP10 | DAP12 | FcεRIβ |
| ASP | DAP10 | DAP12 | FcεRIγ |
| ASP | DAP10 | DAP12 | DAP10 |
| ASP | DAP10 | DAP12 | DAP12 |
| ASP | DAP10 | DAP12 | CD32 |
| ASP | DAP10 | DAP12 | CD79a |
| ASP | DAP10 | DAP12 | CD79b |
| ASP | DAP10 | MyD88 | CD8 |
| ASP | DAP10 | MyD88 | CD3ζ |
| ASP | DAP10 | MyD88 | CD3δ |
| ASP | DAP10 | MyD88 | CD3γ |
| ASP | DAP10 | MyD88 | CD3ε |
| ASP | DAP10 | MyD88 | FcγRI-γ |
| ASP | DAP10 | MyD88 | FcγRIII-γ |
| ASP | DAP10 | MyD88 | FcεRIβ |
| ASP | DAP10 | MyD88 | FcεRIγ |
| ASP | DAP10 | MyD88 | DAP10 |
| ASP | DAP10 | MyD88 | DAP12 |
| ASP | DAP10 | MyD88 | CD32 |
| ASP | DAP10 | MyD88 | CD79a |
| ASP | DAP10 | MyD88 | CD79b |
| ASP | DAP10 | CD7 | CD8 |
| ASP | DAP10 | CD7 | CD3ζ |
| ASP | DAP10 | CD7 | CD3δ |
| ASP | DAP10 | CD7 | CD3γ |
| ASP | DAP10 | CD7 | CD3ε |
| ASP | DAP10 | CD7 | FcγRI-γ |
| ASP | DAP10 | CD7 | FcγRIII-γ |
| ASP | DAP10 | CD7 | FcεRIβ |
| ASP | DAP10 | CD7 | FcεRIγ |
| ASP | DAP10 | CD7 | DAP10 |
| ASP | DAP10 | CD7 | DAP12 |
| ASP | DAP10 | CD7 | CD32 |
| ASP | DAP10 | CD7 | CD79a |
| ASP | DAP10 | CD7 | CD79b |
| ASP | DAP10 | BTNL3 | CD8 |
| ASP | DAP10 | BTNL3 | CD3ζ |
| ASP | DAP10 | BTNL3 | CD3δ |
| ASP | DAP10 | BTNL3 | CD3γ |
| ASP | DAP10 | BTNL3 | CD3ε |
| ASP | DAP10 | BTNL3 | FcγRI-γ |
| ASP | DAP10 | BTNL3 | FcγRIII-γ |
| ASP | DAP10 | BTNL3 | FcεRIβ |
| ASP | DAP10 | BTNL3 | FcεRIγ |
| ASP | DAP10 | BTNL3 | DAP10 |
| ASP | DAP10 | BTNL3 | DAP12 |
| ASP | DAP10 | BTNL3 | CD32 |
| ASP | DAP10 | BTNL3 | CD79a |
| ASP | DAP10 | BTNL3 | CD79b |
| ASP | DAP10 | NKG2D | CD8 |
| ASP | DAP10 | NKG2D | CD3ζ |
| ASP | DAP10 | NKG2D | CD3δ |
| ASP | DAP10 | NKG2D | CD3γ |
| ASP | DAP10 | NKG2D | CD3ε |
| ASP | DAP10 | NKG2D | FcγRI-γ |
| ASP | DAP10 | NKG2D | FcγRIII-γ |
| ASP | DAP10 | NKG2D | FcεRIβ |
| ASP | DAP10 | NKG2D | FcεRIγ |
| ASP | DAP10 | NKG2D | DAP10 |
| ASP | DAP10 | NKG2D | DAP12 |
| ASP | DAP10 | NKG2D | CD32 |
| ASP | DAP10 | NKG2D | CD79a |
| ASP | DAP10 | NKG2D | CD79b |
| ASP | DAP12 | CD28 | CD8 |
| ASP | DAP12 | CD28 | CD3ζ |
| ASP | DAP12 | CD28 | CD3δ |
| ASP | DAP12 | CD28 | CD3γ |
| ASP | DAP12 | CD28 | CD3ε |
| ASP | DAP12 | CD28 | FcγRI-γ |
| ASP | DAP12 | CD28 | FcγRIII-γ |
| ASP | DAP12 | CD28 | FcεRIβ |
| ASP | DAP12 | CD28 | FcεRIγ |
| ASP | DAP12 | CD28 | DAP10 |
| ASP | DAP12 | CD28 | DAP12 |
| ASP | DAP12 | CD28 | CD32 |
| ASP | DAP12 | CD28 | CD79a |
| ASP | DAP12 | CD28 | CD79b |
| ASP | DAP12 | CD8 | CD8 |
| ASP | DAP12 | CD8 | CD3ζ |
| ASP | DAP12 | CD8 | CD3δ |
| ASP | DAP12 | CD8 | CD3γ |
| ASP | DAP12 | CD8 | CD3ε |
| ASP | DAP12 | CD8 | FcγRI-γ |
| ASP | DAP12 | CD8 | FcγRIII-γ |
| ASP | DAP12 | CD8 | FcεRIβ |
| ASP | DAP12 | CD8 | FcεRIγ |
| ASP | DAP12 | CD8 | DAP10 |
| ASP | DAP12 | CD8 | DAP12 |
| ASP | DAP12 | CD8 | CD32 |
| ASP | DAP12 | CD8 | CD79a |
| ASP | DAP12 | CD8 | CD79b |
| ASP | DAP12 | CD4 | CD8 |
| ASP | DAP12 | CD4 | CD3ζ |
| ASP | DAP12 | CD4 | CD3δ |
| ASP | DAP12 | CD4 | CD3γ |
| ASP | DAP12 | CD4 | CD3ε |
| ASP | DAP12 | CD4 | FcγRI-γ |
| ASP | DAP12 | CD4 | FcγRIII-γ |
| ASP | DAP12 | CD4 | FcεRIβ |
| ASP | DAP12 | CD4 | FcεRIγ |
| ASP | DAP12 | CD4 | DAP10 |
| ASP | DAP12 | CD4 | DAP12 |
| ASP | DAP12 | CD4 | CD32 |
| ASP | DAP12 | CD4 | CD79a |
| ASP | DAP12 | CD4 | CD79b |
| ASP | DAP12 | b2c | CD8 |
| ASP | DAP12 | b2c | CD3ζ |
| ASP | DAP12 | b2c | CD3δ |
| ASP | DAP12 | b2c | CD3γ |
| ASP | DAP12 | b2c | CD3ε |
| ASP | DAP12 | b2c | FcγRI-γ |
| ASP | DAP12 | b2c | FcγRIII-γ |
| ASP | DAP12 | b2c | FcεRIβ |
| ASP | DAP12 | b2c | FcεRIγ |
| ASP | DAP12 | b2c | DAP10 |
| ASP | DAP12 | b2c | DAP12 |
| ASP | DAP12 | b2c | CD32 |
| ASP | DAP12 | b2c | CD79a |
| ASP | DAP12 | b2c | CD79b |
| ASP | DAP12 | CD137/41BB | CD8 |
| ASP | DAP12 | CD137/41BB | CD3ζ |
| ASP | DAP12 | CD137/41BB | CD3δ |
| ASP | DAP12 | CD137/41BB | CD3γ |
| ASP | DAP12 | CD137/41BB | CD3ε |
| ASP | DAP12 | CD137/41BB | FcγRI-γ |
| ASP | DAP12 | CD137/41BB | FcγRIII-γ |
| ASP | DAP12 | CD137/41BB | FcεRIβ |
| ASP | DAP12 | CD137/41BB | FcεRIγ |
| ASP | DAP12 | CD137/41BB | DAP10 |
| ASP | DAP12 | CD137/41BB | DAP12 |
| ASP | DAP12 | CD137/41BB | CD32 |
| ASP | DAP12 | CD137/41BB | CD79a |
| ASP | DAP12 | CD137/41BB | CD79b |
| ASP | DAP12 | ICOS | CD8 |
| ASP | DAP12 | ICOS | CD3ζ |
| ASP | DAP12 | ICOS | CD3δ |
| ASP | DAP12 | ICOS | CD3γ |
| ASP | DAP12 | ICOS | CD3ε |
| ASP | DAP12 | ICOS | FcγRI-γ |
| ASP | DAP12 | ICOS | FcγRIII-γ |
| ASP | DAP12 | ICOS | FcεRIβ |
| ASP | DAP12 | ICOS | FcεRIγ |
| ASP | DAP12 | ICOS | DAP10 |
| ASP | DAP12 | ICOS | DAP12 |
| ASP | DAP12 | ICOS | CD32 |
| ASP | DAP12 | ICOS | CD79a |
| ASP | DAP12 | ICOS | CD79b |
| ASP | DAP12 | CD27 | CD8 |
| ASP | DAP12 | CD27 | CD3ζ |
| ASP | DAP12 | CD27 | CD3δ |
| ASP | DAP12 | CD27 | CD3γ |
| ASP | DAP12 | CD27 | CD3ε |
| ASP | DAP12 | CD27 | FcγRI-γ |
| ASP | DAP12 | CD27 | FcγRIII-γ |
| ASP | DAP12 | CD27 | FcεRIβ |
| ASP | DAP12 | CD27 | FcεRIγ |
| ASP | DAP12 | CD27 | DAP10 |
| ASP | DAP12 | CD27 | DAP12 |
| ASP | DAP12 | CD27 | CD32 |
| ASP | DAP12 | CD27 | CD79a |
| ASP | DAP12 | CD27 | CD79b |
| ASP | DAP12 | CD28δ | CD8 |
| ASP | DAP12 | CD28δ | CD3ζ |
| ASP | DAP12 | CD28δ | CD3δ |
| ASP | DAP12 | CD28δ | CD3γ |
| ASP | DAP12 | CD28δ | CD3ε |
| ASP | DAP12 | CD28δ | FcγRI-γ |
| ASP | DAP12 | CD28δ | FcγRIII-γ |
| ASP | DAP12 | CD28δ | FcεRIβ |
| ASP | DAP12 | CD28δ | FcεRIγ |
| ASP | DAP12 | CD28δ | DAP10 |
| ASP | DAP12 | CD28δ | DAP12 |
| ASP | DAP12 | CD28δ | CD32 |
| ASP | DAP12 | CD28δ | CD79a |
| ASP | DAP12 | CD28δ | CD79b |
| ASP | DAP12 | CD80 | CD8 |
| ASP | DAP12 | CD80 | CD3ζ |
| ASP | DAP12 | CD80 | CD3δ |
| ASP | DAP12 | CD80 | CD3γ |
| ASP | DAP12 | CD80 | CD3ε |
| ASP | DAP12 | CD80 | FcγRI-γ |
| ASP | DAP12 | CD80 | FcγRIII-γ |
| ASP | DAP12 | CD80 | FcεRIβ |
| ASP | DAP12 | CD80 | FcεRIγ |
| ASP | DAP12 | CD80 | DAP10 |
| ASP | DAP12 | CD80 | DAP12 |
| ASP | DAP12 | CD80 | CD32 |
| ASP | DAP12 | CD80 | CD79a |
| ASP | DAP12 | CD80 | CD79b |
| ASP | DAP12 | CD86 | CD8 |
| ASP | DAP12 | CD86 | CD3ζ |
| ASP | DAP12 | CD86 | CD3δ |
| ASP | DAP12 | CD86 | CD3γ |
| ASP | DAP12 | CD86 | CD3ε |
| ASP | DAP12 | CD86 | FcγRI-γ |
| ASP | DAP12 | CD86 | FcγRIII-γ |
| ASP | DAP12 | CD86 | FcεRIβ |
| ASP | DAP12 | CD86 | FcεRIγ |
| ASP | DAP12 | CD86 | DAP10 |
| ASP | DAP12 | CD86 | DAP12 |
| ASP | DAP12 | CD86 | CD32 |
| ASP | DAP12 | CD86 | CD79a |
| ASP | DAP12 | CD86 | CD79b |
| ASP | DAP12 | OX40 | CD8 |
| ASP | DAP12 | OX40 | CD3ζ |
| ASP | DAP12 | OX40 | CD3δ |
| ASP | DAP12 | OX40 | CD3γ |
| ASP | DAP12 | OX40 | CD3ε |
| ASP | DAP12 | OX40 | FcγRI-γ |
| ASP | DAP12 | OX40 | FcγRIII-γ |
| ASP | DAP12 | OX40 | FcεRIβ |
| ASP | DAP12 | OX40 | FcεRIγ |
| ASP | DAP12 | OX40 | DAP10 |
| ASP | DAP12 | OX40 | DAP12 |
| ASP | DAP12 | OX40 | CD32 |
| ASP | DAP12 | OX40 | CD79a |
| ASP | DAP12 | OX40 | CD79b |
| ASP | DAP12 | DAP10 | CD8 |
| ASP | DAP12 | DAP10 | CD3ζ |
| ASP | DAP12 | DAP10 | CD3δ |
| ASP | DAP12 | DAP10 | CD3γ |
| ASP | DAP12 | DAP10 | CD3ε |
| ASP | DAP12 | DAP10 | FcγRI-γ |
| ASP | DAP12 | DAP10 | FcγRIII-γ |
| ASP | DAP12 | DAP10 | FcεRIβ |
| ASP | DAP12 | DAP10 | FcεRIγ |
| ASP | DAP12 | DAP10 | DAP10 |
| ASP | DAP12 | DAP10 | DAP12 |
| ASP | DAP12 | DAP10 | CD32 |
| ASP | DAP12 | DAP10 | CD79a |
| ASP | DAP12 | DAP10 | CD79b |
| ASP | DAP12 | DAP12 | CD8 |
| ASP | DAP12 | DAP12 | CD3ζ |
| ASP | DAP12 | DAP12 | CD3δ |
| ASP | DAP12 | DAP12 | CD3γ |
| ASP | DAP12 | DAP12 | CD3ε |
| ASP | DAP12 | DAP12 | FcγRI-γ |
| ASP | DAP12 | DAP12 | FcγRIII-γ |
| ASP | DAP12 | DAP12 | FcεRIβ |
| ASP | DAP12 | DAP12 | FcεRIγ |
| ASP | DAP12 | DAP12 | DAP10 |
| ASP | DAP12 | DAP12 | DAP12 |
| ASP | DAP12 | DAP12 | CD32 |
| ASP | DAP12 | DAP12 | CD79a |
| ASP | DAP12 | DAP12 | CD79b |
| ASP | DAP12 | MyD88 | CD8 |
| ASP | DAP12 | MyD88 | CD3ζ |
| ASP | DAP12 | MyD88 | CD3δ |
| ASP | DAP12 | MyD88 | CD3γ |
| ASP | DAP12 | MyD88 | CD3ε |
| ASP | DAP12 | MyD88 | FcγRI-γ |
| ASP | DAP12 | MyD88 | FcγRIII-γ |
| ASP | DAP12 | MyD88 | FcεRIβ |
| ASP | DAP12 | MyD88 | FcεRIγ |
| ASP | DAP12 | MyD88 | DAP10 |
| ASP | DAP12 | MyD88 | DAP12 |
| ASP | DAP12 | MyD88 | CD32 |
| ASP | DAP12 | MyD88 | CD79a |
| ASP | DAP12 | MyD88 | CD79b |
| ASP | DAP12 | CD7 | CD8 |
| ASP | DAP12 | CD7 | CD3ζ |
| ASP | DAP12 | CD7 | CD3δ |
| ASP | DAP12 | CD7 | CD3γ |
| ASP | DAP12 | CD7 | CD3ε |
| ASP | DAP12 | CD7 | FcγRI-γ |
| ASP | DAP12 | CD7 | FcγRIII-γ |
| ASP | DAP12 | CD7 | FcεRIβ |
| ASP | DAP12 | CD7 | FcεRIγ |
| ASP | DAP12 | CD7 | DAP10 |
| ASP | DAP12 | CD7 | DAP12 |
| ASP | DAP12 | CD7 | CD32 |
| ASP | DAP12 | CD7 | CD79a |
| ASP | DAP12 | CD7 | CD79b |
| ASP | DAP12 | BTNL3 | CD8 |
| ASP | DAP12 | BTNL3 | CD3ζ |
| ASP | DAP12 | BTNL3 | CD3δ |
| ASP | DAP12 | BTNL3 | CD3γ |
| ASP | DAP12 | BTNL3 | CD3ε |
| ASP | DAP12 | BTNL3 | FcγRI-γ |
| ASP | DAP12 | BTNL3 | FcγRIII-γ |
| ASP | DAP12 | BTNL3 | FcεRIβ |
| ASP | DAP12 | BTNL3 | FcεRIγ |
| ASP | DAP12 | BTNL3 | DAP10 |
| ASP | DAP12 | BTNL3 | DAP12 |
| ASP | DAP12 | BTNL3 | CD32 |
| ASP | DAP12 | BTNL3 | CD79a |
| ASP | DAP12 | BTNL3 | CD79b |
| ASP | DAP12 | NKG2D | CD8 |
| ASP | DAP12 | NKG2D | CD3ζ |
| ASP | DAP12 | NKG2D | CD3δ |
| ASP | DAP12 | NKG2D | CD3γ |
| ASP | DAP12 | NKG2D | CD3ε |
| ASP | DAP12 | NKG2D | FcγRI-γ |
| ASP | DAP12 | NKG2D | FcγRIII-γ |
| ASP | DAP12 | NKG2D | FcεRIβ |
| ASP | DAP12 | NKG2D | FcεRIγ |
| ASP | DAP12 | NKG2D | DAP10 |
| ASP | DAP12 | NKG2D | DAP12 |
| ASP | DAP12 | NKG2D | CD32 |
| ASP | DAP12 | NKG2D | CD79a |
| ASP | DAP12 | NKG2D | CD79b |
| ASP | MyD88 | CD28 | CD8 |
| ASP | MyD88 | CD28 | CD3ζ |
| ASP | MyD88 | CD28 | CD3δ |
| ASP | MyD88 | CD28 | CD3γ |
| ASP | MyD88 | CD28 | CD3ε |
| ASP | MyD88 | CD28 | FcγRI-γ |
| ASP | MyD88 | CD28 | FcγRIII-γ |
| ASP | MyD88 | CD28 | FcεRIβ |
| ASP | MyD88 | CD28 | FcεRIγ |
| ASP | MyD88 | CD28 | DAP10 |
| ASP | MyD88 | CD28 | DAP12 |
| ASP | MyD88 | CD28 | CD32 |
| ASP | MyD88 | CD28 | CD79a |
| ASP | MyD88 | CD28 | CD79b |
| ASP | MyD88 | CD8 | CD8 |
| ASP | MyD88 | CD8 | CD3ζ |
| ASP | MyD88 | CD8 | CD3δ |
| ASP | MyD88 | CD8 | CD3γ |
| ASP | MyD88 | CD8 | CD3ε |
| ASP | MyD88 | CD8 | FcγRI-γ |
| ASP | MyD88 | CD8 | FcγRIII-γ |
| ASP | MyD88 | CD8 | FcεRIβ |
| ASP | MyD88 | CD8 | FcεRIγ |
| ASP | MyD88 | CD8 | DAP10 |
| ASP | MyD88 | CD8 | DAP12 |
| ASP | MyD88 | CD8 | CD32 |
| ASP | MyD88 | CD8 | CD79a |
| ASP | MyD88 | CD8 | CD79b |
| ASP | MyD88 | CD4 | CD8 |
| ASP | MyD88 | CD4 | CD3ζ |
| ASP | MyD88 | CD4 | CD3δ |
| ASP | MyD88 | CD4 | CD3γ |
| ASP | MyD88 | CD4 | CD3ε |
| ASP | MyD88 | CD4 | FcγRI-γ |
| ASP | MyD88 | CD4 | FcγRIII-γ |
| ASP | MyD88 | CD4 | FcεRIβ |
| ASP | MyD88 | CD4 | FcεRIγ |
| ASP | MyD88 | CD4 | DAP10 |
| ASP | MyD88 | CD4 | DAP12 |
| ASP | MyD88 | CD4 | CD32 |
| ASP | MyD88 | CD4 | CD79a |
| ASP | MyD88 | CD4 | CD79b |
| ASP | MyD88 | b2c | CD8 |
| ASP | MyD88 | b2c | CD3ζ |
| ASP | MyD88 | b2c | CD3δ |
| ASP | MyD88 | b2c | CD3γ |
| ASP | MyD88 | b2c | CD3ε |
| ASP | MyD88 | b2c | FcγRI-γ |
| ASP | MyD88 | b2c | FcγRIII-γ |
| ASP | MyD88 | b2c | FcεRIβ |
| ASP | MyD88 | b2c | FcεRIγ |
| ASP | MyD88 | b2c | DAP10 |
| ASP | MyD88 | b2c | DAP12 |
| ASP | MyD88 | b2c | CD32 |
| ASP | MyD88 | b2c | CD79a |
| ASP | MyD88 | b2c | CD79b |
| ASP | MyD88 | CD137/41BB | CD8 |
| ASP | MyD88 | CD137/41BB | CD3ζ |
| ASP | MyD88 | CD137/41BB | CD3δ |
| ASP | MyD88 | CD137/41BB | CD3γ |
| ASP | MyD88 | CD137/41BB | CD3ε |
| ASP | MyD88 | CD137/41BB | FcγRI-γ |
| ASP | MyD88 | CD137/41BB | FcγRIII-γ |
| ASP | MyD88 | CD137/41BB | FcεRIβ |
| ASP | MyD88 | CD137/41BB | FcεRIγ |
| ASP | MyD88 | CD137/41BB | DAP10 |
| ASP | MyD88 | CD137/41BB | DAP12 |
| ASP | MyD88 | CD137/41BB | CD32 |
| ASP | MyD88 | CD137/41BB | CD79a |
| ASP | MyD88 | CD137/41BB | CD79b |
| ASP | MyD88 | ICOS | CD8 |
| ASP | MyD88 | ICOS | CD3ζ |
| ASP | MyD88 | ICOS | CD3δ |
| ASP | MyD88 | ICOS | CD3γ |
| ASP | MyD88 | ICOS | CD3ε |
| ASP | MyD88 | ICOS | FcγRI-γ |
| ASP | MyD88 | ICOS | FcγRIII-γ |
| ASP | MyD88 | ICOS | FcεRIβ |
| ASP | MyD88 | ICOS | FcεRIγ |
| ASP | MyD88 | ICOS | DAP10 |
| ASP | MyD88 | ICOS | DAP12 |
| ASP | MyD88 | ICOS | CD32 |
| ASP | MyD88 | ICOS | CD79a |
| ASP | MyD88 | ICOS | CD79b |
| ASP | MyD88 | CD27 | CD8 |
| ASP | MyD88 | CD27 | CD3ζ |
| ASP | MyD88 | CD27 | CD3δ |
| ASP | MyD88 | CD27 | CD3γ |
| ASP | MyD88 | CD27 | CD3ε |
| ASP | MyD88 | CD27 | FcγRI-γ |
| ASP | MyD88 | CD27 | FcγRIII-γ |
| ASP | MyD88 | CD27 | FcεRIβ |
| ASP | MyD88 | CD27 | FcεRIγ |
| ASP | MyD88 | CD27 | DAP10 |
| ASP | MyD88 | CD27 | DAP12 |
| ASP | MyD88 | CD27 | CD32 |
| ASP | MyD88 | CD27 | CD79a |
| ASP | MyD88 | CD27 | CD79b |
| ASP | MyD88 | CD28δ | CD8 |
| ASP | MyD88 | CD28δ | CD3ζ |
| ASP | MyD88 | CD28δ | CD3δ |
| ASP | MyD88 | CD28δ | CD3γ |
| ASP | MyD88 | CD28δ | CD3ε |
| ASP | MyD88 | CD28δ | FcγRI-γ |
| ASP | MyD88 | CD28δ | FcγRIII-γ |
| ASP | MyD88 | CD28δ | FcεRIβ |
| ASP | MyD88 | CD28δ | FcεRIγ |
| ASP | MyD88 | CD28δ | DAP10 |
| ASP | MyD88 | CD28δ | DAP12 |
| ASP | MyD88 | CD28δ | CD32 |
| ASP | MyD88 | CD28δ | CD79a |
| ASP | MyD88 | CD28δ | CD79b |
| ASP | MyD88 | CD80 | CD8 |
| ASP | MyD88 | CD80 | CD3ζ |
| ASP | MyD88 | CD80 | CD3δ |
| ASP | MyD88 | CD80 | CD3γ |
| ASP | MyD88 | CD80 | CD3ε |
| ASP | MyD88 | CD80 | FcγRI-γ |
| ASP | MyD88 | CD80 | FcγRIII-γ |
| ASP | MyD88 | CD80 | FcεRIβ |
| ASP | MyD88 | CD80 | FcεRIγ |
| ASP | MyD88 | CD80 | DAP10 |
| ASP | MyD88 | CD80 | DAP12 |
| ASP | MyD88 | CD80 | CD32 |
| ASP | MyD88 | CD80 | CD79a |
| ASP | MyD88 | CD80 | CD79b |
| ASP | MyD88 | CD86 | CD8 |
| ASP | MyD88 | CD86 | CD3ζ |
| ASP | MyD88 | CD86 | CD3δ |
| ASP | MyD88 | CD86 | CD3γ |
| ASP | MyD88 | CD86 | CD3ε |
| ASP | MyD88 | CD86 | FcγRI-γ |
| ASP | MyD88 | CD86 | FcγRIII-γ |
| ASP | MyD88 | CD86 | FcεRIβ |
| ASP | MyD88 | CD86 | FcεRIγ |
| ASP | MyD88 | CD86 | DAP10 |
| ASP | MyD88 | CD86 | DAP12 |
| ASP | MyD88 | CD86 | CD32 |
| ASP | MyD88 | CD86 | CD79a |
| ASP | MyD88 | CD86 | CD79b |
| ASP | MyD88 | OX40 | CD8 |
| ASP | MyD88 | OX40 | CD3ζ |
| ASP | MyD88 | OX40 | CD3δ |
| ASP | MyD88 | OX40 | CD3γ |
| ASP | MyD88 | OX40 | CD3ε |
| ASP | MyD88 | OX40 | FcγRI-γ |
| ASP | MyD88 | OX40 | FcγRIII-γ |
| ASP | MyD88 | OX40 | FcεRIβ |
| ASP | MyD88 | OX40 | FcεRIγ |
| ASP | MyD88 | OX40 | DAP10 |
| ASP | MyD88 | OX40 | DAP12 |
| ASP | MyD88 | OX40 | CD32 |
| ASP | MyD88 | OX40 | CD79a |
| ASP | MyD88 | OX40 | CD79b |
| ASP | MyD88 | DAP10 | CD8 |
| ASP | MyD88 | DAP10 | CD3ζ |
| ASP | MyD88 | DAP10 | CD3δ |
| ASP | MyD88 | DAP10 | CD3γ |
| ASP | MyD88 | DAP10 | CD3ε |
| ASP | MyD88 | DAP10 | FcγRI-γ |
| ASP | MyD88 | DAP10 | FcγRIII-γ |
| ASP | MyD88 | DAP10 | FcεRIβ |
| ASP | MyD88 | DAP10 | FcεRIγ |
| ASP | MyD88 | DAP10 | DAP10 |
| ASP | MyD88 | DAP10 | DAP12 |
| ASP | MyD88 | DAP10 | CD32 |
| ASP | MyD88 | DAP10 | CD79a |
| ASP | MyD88 | DAP10 | CD79b |
| ASP | MyD88 | DAP12 | CD8 |
| ASP | MyD88 | DAP12 | CD3ζ |
| ASP | MyD88 | DAP12 | CD3δ |
| ASP | MyD88 | DAP12 | CD3γ |
| ASP | MyD88 | DAP12 | CD3ε |
| ASP | MyD88 | DAP12 | FcγRI-γ |
| ASP | MyD88 | DAP12 | FcγRIII-γ |
| ASP | MyD88 | DAP12 | FcεRIβ |
| ASP | MyD88 | DAP12 | FcεRIγ |
| ASP | MyD88 | DAP12 | DAP10 |
| ASP | MyD88 | DAP12 | DAP12 |
| ASP | MyD88 | DAP12 | CD32 |
| ASP | MyD88 | DAP12 | CD79a |
| ASP | MyD88 | DAP12 | CD79b |
| ASP | MyD88 | MyD88 | CD8 |
| ASP | MyD88 | MyD88 | CD3ζ |
| ASP | MyD88 | MyD88 | CD3δ |
| ASP | MyD88 | MyD88 | CD3γ |
| ASP | MyD88 | MyD88 | CD3ε |
| ASP | MyD88 | MyD88 | FcγRI-γ |
| ASP | MyD88 | MyD88 | FcγRIII-γ |
| ASP | MyD88 | MyD88 | FcεRIβ |
| ASP | MyD88 | MyD88 | FcεRIγ |
| ASP | MyD88 | MyD88 | DAP10 |
| ASP | MyD88 | MyD88 | DAP12 |
| ASP | MyD88 | MyD88 | CD32 |
| ASP | MyD88 | MyD88 | CD79a |
| ASP | MyD88 | MyD88 | CD79b |
| ASP | MyD88 | CD7 | CD8 |
| ASP | MyD88 | CD7 | CD3ζ |
| ASP | MyD88 | CD7 | CD3δ |
| ASP | MyD88 | CD7 | CD3γ |
| ASP | MyD88 | CD7 | CD3ε |
| ASP | MyD88 | CD7 | FcγRI-γ |
| ASP | MyD88 | CD7 | FcγRIII-γ |
| ASP | MyD88 | CD7 | FcεRIβ |
| ASP | MyD88 | CD7 | FcεRIγ |
| ASP | MyD88 | CD7 | DAP10 |
| ASP | MyD88 | CD7 | DAP12 |
| ASP | MyD88 | CD7 | CD32 |
| ASP | MyD88 | CD7 | CD79a |
| ASP | MyD88 | CD7 | CD79b |
| ASP | MyD88 | BTNL3 | CD8 |
| ASP | MyD88 | BTNL3 | CD3ζ |
| ASP | MyD88 | BTNL3 | CD3δ |
| ASP | MyD88 | BTNL3 | CD3γ |
| ASP | MyD88 | BTNL3 | CD3ε |
| ASP | MyD88 | BTNL3 | FcγRI-γ |
| ASP | MyD88 | BTNL3 | FcγRIII-γ |
| ASP | MyD88 | BTNL3 | FcεRIβ |
| ASP | MyD88 | BTNL3 | FcεRIγ |
| ASP | MyD88 | BTNL3 | DAP10 |
| ASP | MyD88 | BTNL3 | DAP12 |
| ASP | MyD88 | BTNL3 | CD32 |
| ASP | MyD88 | BTNL3 | CD79a |
| ASP | MyD88 | BTNL3 | CD79b |
| ASP | MyD88 | NKG2D | CD8 |
| ASP | MyD88 | NKG2D | CD3ζ |
| ASP | MyD88 | NKG2D | CD3δ |
| ASP | MyD88 | NKG2D | CD3γ |
| ASP | MyD88 | NKG2D | CD3ε |
| ASP | MyD88 | NKG2D | FcγRI-γ |
| ASP | MyD88 | NKG2D | FcγRIII-γ |
| ASP | MyD88 | NKG2D | FcεRIβ |
| ASP | MyD88 | NKG2D | FcεRIγ |
| ASP | MyD88 | NKG2D | DAP10 |
| ASP | MyD88 | NKG2D | DAP12 |
| ASP | MyD88 | NKG2D | CD32 |
| ASP | MyD88 | NKG2D | CD79a |
| ASP | MyD88 | NKG2D | CD79b |
| ASP | CD7 | CD28 | CD8 |
| ASP | CD7 | CD28 | CD3ζ |
| ASP | CD7 | CD28 | CD3δ |
| ASP | CD7 | CD28 | CD3γ |
| ASP | CD7 | CD28 | CD3ε |
| ASP | CD7 | CD28 | FcγRI-γ |
| ASP | CD7 | CD28 | FcγRIII-γ |
| ASP | CD7 | CD28 | FcεRIβ |
| ASP | CD7 | CD28 | FcεRIγ |
| ASP | CD7 | CD28 | DAP10 |
| ASP | CD7 | CD28 | DAP12 |
| ASP | CD7 | CD28 | CD32 |
| ASP | CD7 | CD28 | CD79a |
| ASP | CD7 | CD28 | CD79b |
| ASP | CD7 | CD8 | CD8 |
| ASP | CD7 | CD8 | CD3ζ |
| ASP | CD7 | CD8 | CD3δ |
| ASP | CD7 | CD8 | CD3γ |
| ASP | CD7 | CD8 | CD3ε |
| ASP | CD7 | CD8 | FcγRI-γ |
| ASP | CD7 | CD8 | FcγRIII-γ |
| ASP | CD7 | CD8 | FcεRIβ |
| ASP | CD7 | CD8 | FcεRIγ |
| ASP | CD7 | CD8 | DAP10 |
| ASP | CD7 | CD8 | DAP12 |
| ASP | CD7 | CD8 | CD32 |
| ASP | CD7 | CD8 | CD79a |
| ASP | CD7 | CD8 | CD79b |
| ASP | CD7 | CD4 | CD8 |
| ASP | CD7 | CD4 | CD3ζ |
| ASP | CD7 | CD4 | CD3δ |
| ASP | CD7 | CD4 | CD3γ |
| ASP | CD7 | CD4 | CD3ε |
| ASP | CD7 | CD4 | FcγRI-γ |
| ASP | CD7 | CD4 | FcγRIII-γ |
| ASP | CD7 | CD4 | FcεRIβ |
| ASP | CD7 | CD4 | FcεRIγ |
| ASP | CD7 | CD4 | DAP10 |
| ASP | CD7 | CD4 | DAP12 |
| ASP | CD7 | CD4 | CD32 |
| ASP | CD7 | CD4 | CD79a |
| ASP | CD7 | CD4 | CD79b |
| ASP | CD7 | b2c | CD8 |
| ASP | CD7 | b2c | CD3ζ |
| ASP | CD7 | b2c | CD3δ |
| ASP | CD7 | b2c | CD3γ |
| ASP | CD7 | b2c | CD3ε |
| ASP | CD7 | b2c | FcγRI-γ |
| ASP | CD7 | b2c | FcγRIII-γ |
| ASP | CD7 | b2c | FcεRIβ |
| ASP | CD7 | b2c | FcεRIγ |
| ASP | CD7 | b2c | DAP10 |
| ASP | CD7 | b2c | DAP12 |
| ASP | CD7 | b2c | CD32 |
| ASP | CD7 | b2c | CD79a |
| ASP | CD7 | b2c | CD79b |
| ASP | CD7 | CD137/41BB | CD8 |
| ASP | CD7 | CD137/41BB | CD3ζ |
| ASP | CD7 | CD137/41BB | CD3δ |
| ASP | CD7 | CD137/41BB | CD3γ |
| ASP | CD7 | CD137/41BB | CD3ε |
| ASP | CD7 | CD137/41BB | FcγRI-γ |
| ASP | CD7 | CD137/41BB | FcγRIII-γ |
| ASP | CD7 | CD137/41BB | FcεRIβ |
| ASP | CD7 | CD137/41BB | FcεRIγ |
| ASP | CD7 | CD137/41BB | DAP10 |
| ASP | CD7 | CD137/41BB | DAP12 |
| ASP | CD7 | CD137/41BB | CD32 |
| ASP | CD7 | CD137/41BB | CD79a |
| ASP | CD7 | CD137/41BB | CD79b |
| ASP | CD7 | ICOS | CD8 |
| ASP | CD7 | ICOS | CD3ζ |
| ASP | CD7 | ICOS | CD3δ |
| ASP | CD7 | ICOS | CD3γ |
| ASP | CD7 | ICOS | CD3ε |
| ASP | CD7 | ICOS | FcγRI-γ |
| ASP | CD7 | ICOS | FcγRIII-γ |
| ASP | CD7 | ICOS | FcεRIβ |
| ASP | CD7 | ICOS | FcεRIγ |
| ASP | CD7 | ICOS | DAP10 |
| ASP | CD7 | ICOS | DAP12 |
| ASP | CD7 | ICOS | CD32 |
| ASP | CD7 | ICOS | CD79a |
| ASP | CD7 | ICOS | CD79b |
| ASP | CD7 | CD27 | CD8 |
| ASP | CD7 | CD27 | CD3ζ |
| ASP | CD7 | CD27 | CD3δ |
| ASP | CD7 | CD27 | CD3γ |
| ASP | CD7 | CD27 | CD3ε |
| ASP | CD7 | CD27 | FcγRI-γ |
| ASP | CD7 | CD27 | FcγRIII-γ |
| ASP | CD7 | CD27 | FcεRIβ |
| ASP | CD7 | CD27 | FcεRIγ |
| ASP | CD7 | CD27 | DAP10 |
| ASP | CD7 | CD27 | DAP12 |
| ASP | CD7 | CD27 | CD32 |
| ASP | CD7 | CD27 | CD79a |
| ASP | CD7 | CD27 | CD79b |
| ASP | CD7 | CD28δ | CD8 |
| ASP | CD7 | CD28δ | CD3ζ |
| ASP | CD7 | CD28δ | CD3δ |
| ASP | CD7 | CD28δ | CD3γ |
| ASP | CD7 | CD28δ | CD3ε |
| ASP | CD7 | CD28δ | FcγRI-γ |
| ASP | CD7 | CD28δ | FcγRIII-γ |
| ASP | CD7 | CD28δ | FcεRIβ |
| ASP | CD7 | CD28δ | FcεRIγ |
| ASP | CD7 | CD28δ | DAP10 |
| ASP | CD7 | CD28δ | DAP12 |
| ASP | CD7 | CD28δ | CD32 |
| ASP | CD7 | CD28δ | CD79a |
| ASP | CD7 | CD28δ | CD79b |
| ASP | CD7 | CD80 | CD8 |
| ASP | CD7 | CD80 | CD3ζ |
| ASP | CD7 | CD80 | CD3δ |
| ASP | CD7 | CD80 | CD3γ |
| ASP | CD7 | CD80 | CD3ε |
| ASP | CD7 | CD80 | FcγRI-γ |
| ASP | CD7 | CD80 | FcγRIII-γ |
| ASP | CD7 | CD80 | FcεRIβ |
| ASP | CD7 | CD80 | FcεRIγ |
| ASP | CD7 | CD80 | DAP10 |
| ASP | CD7 | CD80 | DAP12 |
| ASP | CD7 | CD80 | CD32 |
| ASP | CD7 | CD80 | CD79a |
| ASP | CD7 | CD80 | CD79b |
| ASP | CD7 | CD86 | CD8 |
| ASP | CD7 | CD86 | CD3ζ |
| ASP | CD7 | CD86 | CD3δ |
| ASP | CD7 | CD86 | CD3γ |
| ASP | CD7 | CD86 | CD3ε |
| ASP | CD7 | CD86 | FcγRI-γ |
| ASP | CD7 | CD86 | FcγRIII-γ |
| ASP | CD7 | CD86 | FcεRIβ |
| ASP | CD7 | CD86 | FcεRIγ |
| ASP | CD7 | CD86 | DAP10 |
| ASP | CD7 | CD86 | DAP12 |
| ASP | CD7 | CD86 | CD32 |
| ASP | CD7 | CD86 | CD79a |
| ASP | CD7 | CD86 | CD79b |
| ASP | CD7 | OX40 | CD8 |
| ASP | CD7 | OX40 | CD3ζ |
| ASP | CD7 | OX40 | CD3δ |
| ASP | CD7 | OX40 | CD3γ |
| ASP | CD7 | OX40 | CD3ε |
| ASP | CD7 | OX40 | FcγRI-γ |
| ASP | CD7 | OX40 | FcγRIII-γ |
| ASP | CD7 | OX40 | FcεRIβ |
| ASP | CD7 | OX40 | FcεRIγ |
| ASP | CD7 | OX40 | DAP10 |
| ASP | CD7 | OX40 | DAP12 |
| ASP | CD7 | OX40 | CD32 |
| ASP | CD7 | OX40 | CD79a |
| ASP | CD7 | OX40 | CD79b |
| ASP | CD7 | DAP10 | CD8 |
| ASP | CD7 | DAP10 | CD3ζ |
| ASP | CD7 | DAP10 | CD3δ |
| ASP | CD7 | DAP10 | CD3γ |
| ASP | CD7 | DAP10 | CD3ε |
| ASP | CD7 | DAP10 | FcγRI-γ |
| ASP | CD7 | DAP10 | FcγRIII-γ |
| ASP | CD7 | DAP10 | FcεRIβ |
| ASP | CD7 | DAP10 | FcεRIγ |
| ASP | CD7 | DAP10 | DAP10 |
| ASP | CD7 | DAP10 | DAP12 |
| ASP | CD7 | DAP10 | CD32 |
| ASP | CD7 | DAP10 | CD79a |
| ASP | CD7 | DAP10 | CD79b |
| ASP | CD7 | DAP12 | CD8 |
| ASP | CD7 | DAP12 | CD3ζ |
| ASP | CD7 | DAP12 | CD3δ |
| ASP | CD7 | DAP12 | CD3γ |
| ASP | CD7 | DAP12 | CD3ε |
| ASP | CD7 | DAP12 | FcγRI-γ |
| ASP | CD7 | DAP12 | FcγRIII-γ |
| ASP | CD7 | DAP12 | FcεRIβ |
| ASP | CD7 | DAP12 | FcεRIγ |
| ASP | CD7 | DAP12 | DAP10 |
| ASP | CD7 | DAP12 | DAP12 |
| ASP | CD7 | DAP12 | CD32 |
| ASP | CD7 | DAP12 | CD79a |
| ASP | CD7 | DAP12 | CD79b |
| ASP | CD7 | MyD88 | CD8 |
| ASP | CD7 | MyD88 | CD3ζ |
| ASP | CD7 | MyD88 | CD3δ |
| ASP | CD7 | MyD88 | CD3γ |
| ASP | CD7 | MyD88 | CD3ε |
| ASP | CD7 | MyD88 | FcγRI-γ |
| ASP | CD7 | MyD88 | FcγRIII-γ |
| ASP | CD7 | MyD88 | FcεRIβ |
| ASP | CD7 | MyD88 | FcεRIγ |
| ASP | CD7 | MyD88 | DAP10 |
| ASP | CD7 | MyD88 | DAP12 |
| ASP | CD7 | MyD88 | CD32 |
| ASP | CD7 | MyD88 | CD79a |
| ASP | CD7 | MyD88 | CD79b |
| ASP | CD7 | CD7 | CD8 |
| ASP | CD7 | CD7 | CD3ζ |
| ASP | CD7 | CD7 | CD3δ |
| ASP | CD7 | CD7 | CD3γ |
| ASP | CD7 | CD7 | CD3ε |
| ASP | CD7 | CD7 | FcγRI-γ |
| ASP | CD7 | CD7 | FcγRIII-γ |
| ASP | CD7 | CD7 | FcεRIβ |
| ASP | CD7 | CD7 | FcεRIγ |
| ASP | CD7 | CD7 | DAP10 |
| ASP | CD7 | CD7 | DAP12 |
| ASP | CD7 | CD7 | CD32 |
| ASP | CD7 | CD7 | CD79a |
| ASP | CD7 | CD7 | CD79b |
| ASP | CD7 | BTNL3 | CD8 |
| ASP | CD7 | BTNL3 | CD3ζ |
| ASP | CD7 | BTNL3 | CD3δ |
| ASP | CD7 | BTNL3 | CD3γ |
| ASP | CD7 | BTNL3 | CD3ε |
| ASP | CD7 | BTNL3 | FcγRI-γ |
| ASP | CD7 | BTNL3 | FcγRIII-γ |
| ASP | CD7 | BTNL3 | FcεRIβ |
| ASP | CD7 | BTNL3 | FcεRIγ |
| ASP | CD7 | BTNL3 | DAP10 |
| ASP | CD7 | BTNL3 | DAP12 |
| ASP | CD7 | BTNL3 | CD32 |
| ASP | CD7 | BTNL3 | CD79a |
| ASP | CD7 | BTNL3 | CD79b |
| ASP | CD7 | NKG2D | CD8 |
| ASP | CD7 | NKG2D | CD3ζ |
| ASP | CD7 | NKG2D | CD3δ |
| ASP | CD7 | NKG2D | CD3γ |
| ASP | CD7 | NKG2D | CD3ε |
| ASP | CD7 | NKG2D | FcγRI-γ |
| ASP | CD7 | NKG2D | FcγRIII-γ |
| ASP | CD7 | NKG2D | FcεRIβ |
| ASP | CD7 | NKG2D | FcεRIγ |
| ASP | CD7 | NKG2D | DAP10 |
| ASP | CD7 | NKG2D | DAP12 |
| ASP | CD7 | NKG2D | CD32 |
| ASP | CD7 | NKG2D | CD79a |
| ASP | CD7 | NKG2D | CD79b |
| ASP | BTNL3 | CD28 | CD8 |
| ASP | BTNL3 | CD28 | CD3ζ |
| ASP | BTNL3 | CD28 | CD3δ |
| ASP | BTNL3 | CD28 | CD3γ |
| ASP | BTNL3 | CD28 | CD3ε |
| ASP | BTNL3 | CD28 | FcγRI-γ |
| ASP | BTNL3 | CD28 | FcγRIII-γ |
| ASP | BTNL3 | CD28 | FcεRIβ |
| ASP | BTNL3 | CD28 | FcεRIγ |
| ASP | BTNL3 | CD28 | DAP10 |
| ASP | BTNL3 | CD28 | DAP12 |
| ASP | BTNL3 | CD28 | CD32 |
| ASP | BTNL3 | CD28 | CD79a |
| ASP | BTNL3 | CD28 | CD79b |
| ASP | BTNL3 | CD8 | CD8 |
| ASP | BTNL3 | CD8 | CD3ζ |
| ASP | BTNL3 | CD8 | CD3δ |
| ASP | BTNL3 | CD8 | CD3γ |
| ASP | BTNL3 | CD8 | CD3ε |
| ASP | BTNL3 | CD8 | FcγRI-γ |
| ASP | BTNL3 | CD8 | FcγRIII-γ |
| ASP | BTNL3 | CD8 | FcεRIβ |
| ASP | BTNL3 | CD8 | FcεRIγ |
| ASP | BTNL3 | CD8 | DAP10 |
| ASP | BTNL3 | CD8 | DAP12 |
| ASP | BTNL3 | CD8 | CD32 |
| ASP | BTNL3 | CD8 | CD79a |
| ASP | BTNL3 | CD8 | CD79b |
| ASP | BTNL3 | CD4 | CD8 |
| ASP | BTNL3 | CD4 | CD3ζ |
| ASP | BTNL3 | CD4 | CD3δ |
| ASP | BTNL3 | CD4 | CD3γ |
| ASP | BTNL3 | CD4 | CD3ε |
| ASP | BTNL3 | CD4 | FcγRI-γ |
| ASP | BTNL3 | CD4 | FcγRIII-γ |
| ASP | BTNL3 | CD4 | FcεRIβ |
| ASP | BTNL3 | CD4 | FcεRIγ |
| ASP | BTNL3 | CD4 | DAP10 |
| ASP | BTNL3 | CD4 | DAP12 |
| ASP | BTNL3 | CD4 | CD32 |
| ASP | BTNL3 | CD4 | CD79a |
| ASP | BTNL3 | CD4 | CD79b |
| ASP | BTNL3 | b2c | CD8 |
| ASP | BTNL3 | b2c | CD3ζ |
| ASP | BTNL3 | b2c | CD3δ |
| ASP | BTNL3 | b2c | CD3γ |
| ASP | BTNL3 | b2c | CD3ε |
| ASP | BTNL3 | b2c | FcγRI-γ |
| ASP | BTNL3 | b2c | FcγRIII-γ |
| ASP | BTNL3 | b2c | FcεRIβ |
| ASP | BTNL3 | b2c | FcεRIγ |
| ASP | BTNL3 | b2c | DAP10 |
| ASP | BTNL3 | b2c | DAP12 |
| ASP | BTNL3 | b2c | CD32 |
| ASP | BTNL3 | b2c | CD79a |
| ASP | BTNL3 | b2c | CD79b |
| ASP | BTNL3 | CD137/41BB | CD8 |
| ASP | BTNL3 | CD137/41BB | CD3ζ |
| ASP | BTNL3 | CD137/41BB | CD3δ |
| ASP | BTNL3 | CD137/41BB | CD3γ |
| ASP | BTNL3 | CD137/41BB | CD3ε |
| ASP | BTNL3 | CD137/41BB | FcγRI-γ |
| ASP | BTNL3 | CD137/41BB | FcγRIII-γ |
| ASP | BTNL3 | CD137/41BB | FcεRIβ |
| ASP | BTNL3 | CD137/41BB | FcεRIγ |
| ASP | BTNL3 | CD137/41BB | DAP10 |
| ASP | BTNL3 | CD137/41BB | DAP12 |
| ASP | BTNL3 | CD137/41BB | CD32 |
| ASP | BTNL3 | CD137/41BB | CD79a |
| ASP | BTNL3 | CD137/41BB | CD79b |
| ASP | BTNL3 | ICOS | CD8 |
| ASP | BTNL3 | ICOS | CD3ζ |
| ASP | BTNL3 | ICOS | CD3δ |
| ASP | BTNL3 | ICOS | CD3γ |
| ASP | BTNL3 | ICOS | CD3ε |
| ASP | BTNL3 | ICOS | FcγRI-γ |
| ASP | BTNL3 | ICOS | FcγRIII-γ |
| ASP | BTNL3 | ICOS | FcεRIβ |
| ASP | BTNL3 | ICOS | FcεRIγ |
| ASP | BTNL3 | ICOS | DAP10 |
| ASP | BTNL3 | ICOS | DAP12 |
| ASP | BTNL3 | ICOS | CD32 |
| ASP | BTNL3 | ICOS | CD79a |
| ASP | BTNL3 | ICOS | CD79b |
| ASP | BTNL3 | CD27 | CD8 |
| ASP | BTNL3 | CD27 | CD3ζ |
| ASP | BTNL3 | CD27 | CD3δ |
| ASP | BTNL3 | CD27 | CD3γ |
| ASP | BTNL3 | CD27 | CD3ε |
| ASP | BTNL3 | CD27 | FcγRI-γ |
| ASP | BTNL3 | CD27 | FcγRIII-γ |
| ASP | BTNL3 | CD27 | FcεRIβ |
| ASP | BTNL3 | CD27 | FcεRIγ |
| ASP | BTNL3 | CD27 | DAP10 |
| ASP | BTNL3 | CD27 | DAP12 |
| ASP | BTNL3 | CD27 | CD32 |
| ASP | BTNL3 | CD27 | CD79a |
| ASP | BTNL3 | CD27 | CD79b |
| ASP | BTNL3 | CD28δ | CD8 |
| ASP | BTNL3 | CD28δ | CD3ζ |
| ASP | BTNL3 | CD28δ | CD3δ |
| ASP | BTNL3 | CD28δ | CD3γ |
| ASP | BTNL3 | CD28δ | CD3ε |
| ASP | BTNL3 | CD28δ | FcγRI-γ |
| ASP | BTNL3 | CD28δ | FcγRIII-γ |
| ASP | BTNL3 | CD28δ | FcεRIβ |
| ASP | BTNL3 | CD28δ | FcεRIγ |
| ASP | BTNL3 | CD28δ | DAP10 |
| ASP | BTNL3 | CD28δ | DAP12 |
| ASP | BTNL3 | CD28δ | CD32 |
| ASP | BTNL3 | CD28δ | CD79a |
| ASP | BTNL3 | CD28δ | CD79b |
| ASP | BTNL3 | CD80 | CD8 |
| ASP | BTNL3 | CD80 | CD3ζ |
| ASP | BTNL3 | CD80 | CD3δ |
| ASP | BTNL3 | CD80 | CD3γ |
| ASP | BTNL3 | CD80 | CD3ε |
| ASP | BTNL3 | CD80 | FcγRI-γ |
| ASP | BTNL3 | CD80 | FcγRIII-γ |
| ASP | BTNL3 | CD80 | FcεRIβ |
| ASP | BTNL3 | CD80 | FcεRIγ |
| ASP | BTNL3 | CD80 | DAP10 |
| ASP | BTNL3 | CD80 | DAP12 |
| ASP | BTNL3 | CD80 | CD32 |
| ASP | BTNL3 | CD80 | CD79a |
| ASP | BTNL3 | CD80 | CD79b |
| ASP | BTNL3 | CD86 | CD8 |
| ASP | BTNL3 | CD86 | CD3ζ |
| ASP | BTNL3 | CD86 | CD3δ |
| ASP | BTNL3 | CD86 | CD3γ |
| ASP | BTNL3 | CD86 | CD3ε |
| ASP | BTNL3 | CD86 | FcγRI-γ |
| ASP | BTNL3 | CD86 | FcγRIII-γ |
| ASP | BTNL3 | CD86 | FcεRIβ |
| ASP | BTNL3 | CD86 | FcεRIγ |
| ASP | BTNL3 | CD86 | DAP10 |
| ASP | BTNL3 | CD86 | DAP12 |
| ASP | BTNL3 | CD86 | CD32 |
| ASP | BTNL3 | CD86 | CD79a |
| ASP | BTNL3 | CD86 | CD79b |
| ASP | BTNL3 | OX40 | CD8 |
| ASP | BTNL3 | OX40 | CD3ζ |
| ASP | BTNL3 | OX40 | CD3δ |
| ASP | BTNL3 | OX40 | CD3γ |
| ASP | BTNL3 | OX40 | CD3ε |
| ASP | BTNL3 | OX40 | FcγRI-γ |
| ASP | BTNL3 | OX40 | FcγRIII-γ |
| ASP | BTNL3 | OX40 | FcεRIβ |
| ASP | BTNL3 | OX40 | FcεRIγ |
| ASP | BTNL3 | OX40 | DAP10 |
| ASP | BTNL3 | OX40 | DAP12 |
| ASP | BTNL3 | OX40 | CD32 |
| ASP | BTNL3 | OX40 | CD79a |
| ASP | BTNL3 | OX40 | CD79b |
| ASP | BTNL3 | DAP10 | CD8 |
| ASP | BTNL3 | DAP10 | CD3ζ |
| ASP | BTNL3 | DAP10 | CD3δ |
| ASP | BTNL3 | DAP10 | CD3γ |
| ASP | BTNL3 | DAP10 | CD3ε |
| ASP | BTNL3 | DAP10 | FcγRI-γ |
| ASP | BTNL3 | DAP10 | FcγRIII-γ |
| ASP | BTNL3 | DAP10 | FcεRIβ |
| ASP | BTNL3 | DAP10 | FcεRIγ |
| ASP | BTNL3 | DAP10 | DAP10 |
| ASP | BTNL3 | DAP10 | DAP12 |
| ASP | BTNL3 | DAP10 | CD32 |
| ASP | BTNL3 | DAP10 | CD79a |
| ASP | BTNL3 | DAP10 | CD79b |
| ASP | BTNL3 | DAP12 | CD8 |
| ASP | BTNL3 | DAP12 | CD3ζ |
| ASP | BTNL3 | DAP12 | CD3δ |
| ASP | BTNL3 | DAP12 | CD3γ |
| ASP | BTNL3 | DAP12 | CD3ε |
| ASP | BTNL3 | DAP12 | FcγRI-γ |
| ASP | BTNL3 | DAP12 | FcγRIII-γ |
| ASP | BTNL3 | DAP12 | FcεRIβ |
| ASP | BTNL3 | DAP12 | FcεRIγ |
| ASP | BTNL3 | DAP12 | DAP10 |
| ASP | BTNL3 | DAP12 | DAP12 |
| ASP | BTNL3 | DAP12 | CD32 |
| ASP | BTNL3 | DAP12 | CD79a |
| ASP | BTNL3 | DAP12 | CD79b |
| ASP | BTNL3 | MyD88 | CD8 |
| ASP | BTNL3 | MyD88 | CD3ζ |
| ASP | BTNL3 | MyD88 | CD3δ |
| ASP | BTNL3 | MyD88 | CD3γ |
| ASP | BTNL3 | MyD88 | CD3ε |
| ASP | BTNL3 | MyD88 | FcγRI-γ |
| ASP | BTNL3 | MyD88 | FcγRIII-γ |
| ASP | BTNL3 | MyD88 | FcεRIβ |
| ASP | BTNL3 | MyD88 | FcεRIγ |
| ASP | BTNL3 | MyD88 | DAP10 |
| ASP | BTNL3 | MyD88 | DAP12 |
| ASP | BTNL3 | MyD88 | CD32 |
| ASP | BTNL3 | MyD88 | CD79a |
| ASP | BTNL3 | MyD88 | CD79b |
| ASP | BTNL3 | CD7 | CD8 |
| ASP | BTNL3 | CD7 | CD3ζ |
| ASP | BTNL3 | CD7 | CD3δ |
| ASP | BTNL3 | CD7 | CD3γ |
| ASP | BTNL3 | CD7 | CD3ε |
| ASP | BTNL3 | CD7 | FcγRI-γ |
| ASP | BTNL3 | CD7 | FcγRIII-γ |
| ASP | BTNL3 | CD7 | FcεRIβ |
| ASP | BTNL3 | CD7 | FcεRIγ |
| ASP | BTNL3 | CD7 | DAP10 |
| ASP | BTNL3 | CD7 | DAP12 |
| ASP | BTNL3 | CD7 | CD32 |
| ASP | BTNL3 | CD7 | CD79a |
| ASP | BTNL3 | CD7 | CD79b |
| ASP | BTNL3 | BTNL3 | CD8 |
| ASP | BTNL3 | BTNL3 | CD3ζ |
| ASP | BTNL3 | BTNL3 | CD3δ |
| ASP | BTNL3 | BTNL3 | CD3γ |
| ASP | BTNL3 | BTNL3 | CD3ε |
| ASP | BTNL3 | BTNL3 | FcγRI-γ |
| ASP | BTNL3 | BTNL3 | FcγRIII-γ |
| ASP | BTNL3 | BTNL3 | FcεRIβ |
| ASP | BTNL3 | BTNL3 | FcεRIγ |
| ASP | BTNL3 | BTNL3 | DAP10 |
| ASP | BTNL3 | BTNL3 | DAP12 |
| ASP | BTNL3 | BTNL3 | CD32 |
| ASP | BTNL3 | BTNL3 | CD79a |
| ASP | BTNL3 | BTNL3 | CD79b |
| ASP | BTNL3 | NKG2D | CD8 |
| ASP | BTNL3 | NKG2D | CD3ζ |
| ASP | BTNL3 | NKG2D | CD3δ |
| ASP | BTNL3 | NKG2D | CD3γ |
| ASP | BTNL3 | NKG2D | CD3ε |
| ASP | BTNL3 | NKG2D | FcγRI-γ |
| ASP | BTNL3 | NKG2D | FcγRIII-γ |
| ASP | BTNL3 | NKG2D | FcεRIβ |
| ASP | BTNL3 | NKG2D | FcεRIγ |
| ASP | BTNL3 | NKG2D | DAP10 |
| ASP | BTNL3 | NKG2D | DAP12 |
| ASP | BTNL3 | NKG2D | CD32 |
| ASP | BTNL3 | NKG2D | CD79a |
| ASP | BTNL3 | NKG2D | CD79b |
| ASP | NKG2D | CD28 | CD8 |
| ASP | NKG2D | CD28 | CD3ζ |
| ASP | NKG2D | CD28 | CD3δ |
| ASP | NKG2D | CD28 | CD3γ |
| ASP | NKG2D | CD28 | CD3ε |
| ASP | NKG2D | CD28 | FcγRI-γ |
| ASP | NKG2D | CD28 | FcγRIII-γ |
| ASP | NKG2D | CD28 | FcεRIβ |
| ASP | NKG2D | CD28 | FcεRIγ |
| ASP | NKG2D | CD28 | DAP10 |
| ASP | NKG2D | CD28 | DAP12 |
| ASP | NKG2D | CD28 | CD32 |
| ASP | NKG2D | CD28 | CD79a |
| ASP | NKG2D | CD28 | CD79b |
| ASP | NKG2D | CD8 | CD8 |
| ASP | NKG2D | CD8 | CD3ζ |
| ASP | NKG2D | CD8 | CD3δ |
| ASP | NKG2D | CD8 | CD3γ |
| ASP | NKG2D | CD8 | CD3ε |
| ASP | NKG2D | CD8 | FcγRI-γ |
| ASP | NKG2D | CD8 | FcγRIII-γ |
| ASP | NKG2D | CD8 | FcεRIβ |
| ASP | NKG2D | CD8 | FcεRIγ |
| ASP | NKG2D | CD8 | DAP10 |
| ASP | NKG2D | CD8 | DAP12 |
| ASP | NKG2D | CD8 | CD32 |
| ASP | NKG2D | CD8 | CD79a |
| ASP | NKG2D | CD8 | CD79b |
| ASP | NKG2D | CD4 | CD8 |
| ASP | NKG2D | CD4 | CD3ζ |
| ASP | NKG2D | CD4 | CD3δ |
| ASP | NKG2D | CD4 | CD3γ |
| ASP | NKG2D | CD4 | CD3ε |
| ASP | NKG2D | CD4 | FcγRI-γ |
| ASP | NKG2D | CD4 | FcγRIII-γ |
| ASP | NKG2D | CD4 | FcεRIβ |
| ASP | NKG2D | CD4 | FcεRIγ |
| ASP | NKG2D | CD4 | DAP10 |
| ASP | NKG2D | CD4 | DAP12 |
| ASP | NKG2D | CD4 | CD32 |
| ASP | NKG2D | CD4 | CD79a |
| ASP | NKG2D | CD4 | CD79b |
| ASP | NKG2D | b2c | CD8 |
| ASP | NKG2D | b2c | CD3ζ |
| ASP | NKG2D | b2c | CD3δ |
| ASP | NKG2D | b2c | CD3γ |
| ASP | NKG2D | b2c | CD3ε |
| ASP | NKG2D | b2c | FcγRI-γ |
| ASP | NKG2D | b2c | FcγRIII-γ |
| ASP | NKG2D | b2c | FcεRIβ |
| ASP | NKG2D | b2c | FcεRIγ |
| ASP | NKG2D | b2c | DAP10 |
| ASP | NKG2D | b2c | DAP12 |
| ASP | NKG2D | b2c | CD32 |
| ASP | NKG2D | b2c | CD79a |
| ASP | NKG2D | b2c | CD79b |
| ASP | NKG2D | CD137/41BB | CD8 |
| ASP | NKG2D | CD137/41BB | CD3ζ |
| ASP | NKG2D | CD137/41BB | CD3δ |
| ASP | NKG2D | CD137/41BB | CD3γ |
| ASP | NKG2D | CD137/41BB | CD3ε |
| ASP | NKG2D | CD137/41BB | FcγRI-γ |
| ASP | NKG2D | CD137/41BB | FcγRIII-γ |
| ASP | NKG2D | CD137/41BB | FcεRIβ |
| ASP | NKG2D | CD137/41BB | FcεRIγ |
| ASP | NKG2D | CD137/41BB | DAP10 |
| ASP | NKG2D | CD137/41BB | DAP12 |
| ASP | NKG2D | CD137/41BB | CD32 |
| ASP | NKG2D | CD137/41BB | CD79a |
| ASP | NKG2D | CD137/41BB | CD79b |
| ASP | NKG2D | ICOS | CD8 |
| ASP | NKG2D | ICOS | CD3ζ |
| ASP | NKG2D | ICOS | CD3δ |
| ASP | NKG2D | ICOS | CD3γ |
| ASP | NKG2D | ICOS | CD3ε |
| ASP | NKG2D | ICOS | FcγRI-γ |
| ASP | NKG2D | ICOS | FcγRIII-γ |
| ASP | NKG2D | ICOS | FcεRIβ |
| ASP | NKG2D | ICOS | FcεRIγ |
| ASP | NKG2D | ICOS | DAP10 |
| ASP | NKG2D | ICOS | DAP12 |
| ASP | NKG2D | ICOS | CD32 |
| ASP | NKG2D | ICOS | CD79a |
| ASP | NKG2D | ICOS | CD79b |
| ASP | NKG2D | CD27 | CD8 |
| ASP | NKG2D | CD27 | CD3ζ |
| ASP | NKG2D | CD27 | CD3δ |
| ASP | NKG2D | CD27 | CD3γ |
| ASP | NKG2D | CD27 | CD3ε |
| ASP | NKG2D | CD27 | FcγRI-γ |
| ASP | NKG2D | CD27 | FcγRIII-γ |
| ASP | NKG2D | CD27 | FcεRIβ |
| ASP | NKG2D | CD27 | FcεRIγ |
| ASP | NKG2D | CD27 | DAP10 |
| ASP | NKG2D | CD27 | DAP12 |
| ASP | NKG2D | CD27 | CD32 |
| ASP | NKG2D | CD27 | CD79a |
| ASP | NKG2D | CD27 | CD79b |
| ASP | NKG2D | CD28δ | CD8 |
| ASP | NKG2D | CD28δ | CD3ζ |
| ASP | NKG2D | CD28δ | CD3δ |
| ASP | NKG2D | CD28δ | CD3γ |
| ASP | NKG2D | CD28δ | CD3ε |
| ASP | NKG2D | CD28δ | FcγRI-γ |
| ASP | NKG2D | CD28δ | FcγRIII-γ |
| ASP | NKG2D | CD28δ | FcεRIβ |
| ASP | NKG2D | CD28δ | FcεRIγ |
| ASP | NKG2D | CD28δ | DAP10 |
| ASP | NKG2D | CD28δ | DAP12 |
| ASP | NKG2D | CD28δ | CD32 |
| ASP | NKG2D | CD28δ | CD79a |
| ASP | NKG2D | CD28δ | CD79b |
| ASP | NKG2D | CD80 | CD8 |
| ASP | NKG2D | CD80 | CD3ζ |
| ASP | NKG2D | CD80 | CD3δ |
| ASP | NKG2D | CD80 | CD3γ |
| ASP | NKG2D | CD80 | CD3ε |
| ASP | NKG2D | CD80 | FcγRI-γ |
| ASP | NKG2D | CD80 | FcγRIII-γ |
| ASP | NKG2D | CD80 | FcεRIβ |
| ASP | NKG2D | CD80 | FcεRIγ |
| ASP | NKG2D | CD80 | DAP10 |
| ASP | NKG2D | CD80 | DAP12 |
| ASP | NKG2D | CD80 | CD32 |
| ASP | NKG2D | CD80 | CD79a |
| ASP | NKG2D | CD80 | CD79b |
| ASP | NKG2D | CD86 | CD8 |
| ASP | NKG2D | CD86 | CD3ζ |
| ASP | NKG2D | CD86 | CD3δ |
| ASP | NKG2D | CD86 | CD3γ |
| ASP | NKG2D | CD86 | CD3ε |
| ASP | NKG2D | CD86 | FcγRI-γ |
| ASP | NKG2D | CD86 | FcγRIII-γ |
| ASP | NKG2D | CD86 | FcεRIβ |
| ASP | NKG2D | CD86 | FcεRIγ |
| ASP | NKG2D | CD86 | DAP10 |
| ASP | NKG2D | CD86 | DAP12 |
| ASP | NKG2D | CD86 | CD32 |
| ASP | NKG2D | CD86 | CD79a |
| ASP | NKG2D | CD86 | CD79b |
| ASP | NKG2D | OX40 | CD8 |
| ASP | NKG2D | OX40 | CD3ζ |
| ASP | NKG2D | OX40 | CD3δ |
| ASP | NKG2D | OX40 | CD3γ |
| ASP | NKG2D | OX40 | CD3ε |
| ASP | NKG2D | OX40 | FcγRI-γ |
| ASP | NKG2D | OX40 | FcγRIII-γ |
| ASP | NKG2D | OX40 | FcεRIβ |
| ASP | NKG2D | OX40 | FcεRIγ |
| ASP | NKG2D | OX40 | DAP10 |
| ASP | NKG2D | OX40 | DAP12 |
| ASP | NKG2D | OX40 | CD32 |
| ASP | NKG2D | OX40 | CD79a |
| ASP | NKG2D | OX40 | CD79b |
| ASP | NKG2D | DAP10 | CD8 |
| ASP | NKG2D | DAP10 | CD3ζ |
| ASP | NKG2D | DAP10 | CD3δ |
| ASP | NKG2D | DAP10 | CD3γ |
| ASP | NKG2D | DAP10 | CD3ε |
| ASP | NKG2D | DAP10 | FcγRI-γ |
| ASP | NKG2D | DAP10 | FcγRIII-γ |
| ASP | NKG2D | DAP10 | FcεRIβ |
| ASP | NKG2D | DAP10 | FcεRIγ |
| ASP | NKG2D | DAP10 | DAP10 |
| ASP | NKG2D | DAP10 | DAP12 |
| ASP | NKG2D | DAP10 | CD32 |
| ASP | NKG2D | DAP10 | CD79a |
| ASP | NKG2D | DAP10 | CD79b |
| ASP | NKG2D | DAP12 | CD8 |
| ASP | NKG2D | DAP12 | CD3ζ |
| ASP | NKG2D | DAP12 | CD3δ |
| ASP | NKG2D | DAP12 | CD3γ |
| ASP | NKG2D | DAP12 | CD3ε |
| ASP | NKG2D | DAP12 | FcγRI-γ |
| ASP | NKG2D | DAP12 | FcγRIII-γ |
| ASP | NKG2D | DAP12 | FcεRIβ |
| ASP | NKG2D | DAP12 | FcεRIγ |
| ASP | NKG2D | DAP12 | DAP10 |
| ASP | NKG2D | DAP12 | DAP12 |
| ASP | NKG2D | DAP12 | CD32 |
| ASP | NKG2D | DAP12 | CD79a |
| ASP | NKG2D | DAP12 | CD79b |
| ASP | NKG2D | MyD88 | CD8 |
| ASP | NKG2D | MyD88 | CD3ζ |
| ASP | NKG2D | MyD88 | CD3δ |
| ASP | NKG2D | MyD88 | CD3γ |
| ASP | NKG2D | MyD88 | CD3ε |
| ASP | NKG2D | MyD88 | FcγRI-γ |
| ASP | NKG2D | MyD88 | FcγRIII-γ |
| ASP | NKG2D | MyD88 | FcεRIβ |
| ASP | NKG2D | MyD88 | FcεRIγ |
| ASP | NKG2D | MyD88 | DAP10 |
| ASP | NKG2D | MyD88 | DAP12 |
| ASP | NKG2D | MyD88 | CD32 |
| ASP | NKG2D | MyD88 | CD79a |
| ASP | NKG2D | MyD88 | CD79b |
| ASP | NKG2D | CD7 | CD8 |
| ASP | NKG2D | CD7 | CD3ζ |
| ASP | NKG2D | CD7 | CD3δ |
| ASP | NKG2D | CD7 | CD3γ |
| ASP | NKG2D | CD7 | CD3ε |
| ASP | NKG2D | CD7 | FcγRI-γ |
| ASP | NKG2D | CD7 | FcγRIII-γ |
| ASP | NKG2D | CD7 | FcεRIβ |
| ASP | NKG2D | CD7 | FcεRIγ |
| ASP | NKG2D | CD7 | DAP10 |
| ASP | NKG2D | CD7 | DAP12 |
| ASP | NKG2D | CD7 | CD32 |
| ASP | NKG2D | CD7 | CD79a |
| ASP | NKG2D | CD7 | CD79b |
| ASP | NKG2D | BTNL3 | CD8 |
| ASP | NKG2D | BTNL3 | CD3ζ |
| ASP | NKG2D | BTNL3 | CD3δ |
| ASP | NKG2D | BTNL3 | CD3γ |
| ASP | NKG2D | BTNL3 | CD3ε |
| ASP | NKG2D | BTNL3 | FcγRI-γ |
| ASP | NKG2D | BTNL3 | FcγRIII-γ |
| ASP | NKG2D | BTNL3 | FcεRIβ |
| ASP | NKG2D | BTNL3 | FcεRIγ |
| ASP | NKG2D | BTNL3 | DAP10 |
| ASP | NKG2D | BTNL3 | DAP12 |
| ASP | NKG2D | BTNL3 | CD32 |
| ASP | NKG2D | BTNL3 | CD79a |
| ASP | NKG2D | BTNL3 | CD79b |
| ASP | NKG2D | NKG2D | CD8 |
| ASP | NKG2D | NKG2D | CD3ζ |
| ASP | NKG2D | NKG2D | CD3δ |
| ASP | NKG2D | NKG2D | CD3γ |
| ASP | NKG2D | NKG2D | CD3ε |
| ASP | NKG2D | NKG2D | FcγRI-γ |
| ASP | NKG2D | NKG2D | FcγRIII-γ |
| ASP | NKG2D | NKG2D | FcεRIβ |
| ASP | NKG2D | NKG2D | FcεRIγ |
| ASP | NKG2D | NKG2D | DAP10 |
| ASP | NKG2D | NKG2D | DAP12 |
| ASP | NKG2D | NKG2D | CD32 |
| ASP | NKG2D | NKG2D | CD79a |
| ASP | NKG2D | NKG2D | CD79b |

Table 4

CARs lacking Co-Stimulatory Signal (for dual CAR approach)

| | | |
|---|---|---|
| ScFv | Co-stimulatory Signal | Signal Domain |
| ASP | none | CD8 |
| ASP | none | CD3ζ |
| ASP | none | CD3δ |
| ASP | none | CD3γ |
| ASP | none | CD3ε |
| ASP | none | FcγRI-γ |
| ASP | none | FcγRIII-γ |
| ASP | none | FcεRIβ |
| ASP | none | FcεRIγ |
| ASP | none | DAP10 |
| ASP | none | DAP12 |
| ASP | none | CD32 |
| ASP | none | CD79a |
| ASP | none | CD8 |
| ASP | none | CD3ζ |
| ASP | none | CD3δ |
| ASP | none | CD3γ |
| ASP | none | CD3ε |
| ASP | none | FcγRI-γ |

Table 5

CARs lacking Signal Domain (for dual CAR approach)

| | | |
|---|---|---|
| ScFv | Co-stimulatory Signal | Signal Domain |
| ASP | CD28 | none |
| ASP | CD8 | none |
| ASP | CD4 | none |
| ASP | b2c | none |
| ASP | CD137/41BB | none |
| ASP | ICOS | none |
| ASP | CD27 | none |
| ASP | CD28δ | none |
| ASP | CD80 | none |
| ASP | CD86 | none |
| ASP | OX40 | none |
| ASP | DAP10 | none |
| ASP | MyD88 | none |
| ASP | CD7 | none |
| ASP | DAP12 | none |
| ASP | MyD88 | none |
| ASP | CD7 | none |
| ASP | BTNL3 | none |
| ASP | NKG2D | none |

Table 6

Third Generation CARs lacking Signal Domain (for dual CAR approach)

| | | | |
|---|---|---|---|
| ScFv | Co-stimulatory Signal | Co-stimulatory Signal | Signal Domain |
| ASP | CD28 | CD28 | none |
| ASP | CD28 | CD8 | none |
| ASP | CD28 | CD4 | none |
| ASP | CD28 | b2c | none |
| ASP | CD28 | CD137/41BB | none |
| ASP | CD28 | ICOS | none |
| ASP | CD28 | CD27 | none |
| ASP | CD28 | CD28δ | none |
| ASP | CD28 | CD80 | none |
| ASP | CD28 | CD86 | none |
| ASP | CD28 | OX40 | none |
| ASP | CD28 | DAP10 | none |
| ASP | CD28 | MyD88 | none |
| ASP | CD28 | CD7 | none |
| ASP | CD28 | DAP12 | none |
| ASP | CD28 | MyD88 | none |
| ASP | CD28 | CD7 | none |
| ASP | CD8 | CD28 | none |
| ASP | CD8 | CD8 | none |
| ASP | CD8 | CD4 | none |
| ASP | CD8 | b2c | none |
| ASP | CD8 | CD137/41BB | none |
| ASP | CD8 | ICOS | none |
| ASP | CD8 | CD27 | none |
| ASP | CD8 | CD28δ | none |
| ASP | CD8 | CD80 | none |
| ASP | CD8 | CD86 | none |
| ASP | CD8 | OX40 | none |
| ASP | CD8 | DAP10 | none |
| ASP | CD8 | MyD88 | none |
| ASP | CD8 | CD7 | none |
| ASP | CD8 | DAP12 | none |
| ASP | CD8 | MyD88 | none |
| ASP | CD8 | CD7 | none |
| ASP | CD4 | CD28 | none |
| ASP | CD4 | CD8 | none |
| ASP | CD4 | CD4 | none |
| ASP | CD4 | b2c | none |
| ASP | CD4 | CD137/41BB | none |
| ASP | CD4 | ICOS | none |
| ASP | CD4 | CD27 | none |
| ASP | CD4 | CD28δ | none |
| ASP | CD4 | CD80 | none |
| ASP | CD4 | CD86 | none |
| ASP | CD4 | OX40 | none |
| ASP | CD4 | DAP10 | none |
| ASP | CD4 | MyD88 | none |
| ASP | CD4 | CD7 | none |
| ASP | CD4 | DAP12 | none |
| ASP | CD4 | MyD88 | none |
| ASP | CD4 | CD7 | none |
| ASP | b2c | CD28 | none |
| ASP | b2c | CD8 | none |
| ASP | b2c | CD4 | none |
| ASP | b2c | b2c | none |
| ASP | b2c | CD137/41BB | none |
| ASP | b2c | ICOS | none |
| ASP | b2c | CD27 | none |
| ASP | b2c | CD28δ | none |
| ASP | b2c | CD80 | none |
| ASP | b2c | CD86 | none |
| ASP | b2c | OX40 | none |
| ASP | b2c | DAP10 | none |
| ASP | b2c | MyD88 | none |
| ASP | b2c | CD7 | none |
| ASP | b2c | DAP12 | none |
| ASP | b2c | MyD88 | none |
| ASP | b2c | CD7 | none |
| ASP | CD137/41BB | CD28 | none |
| ASP | CD137/41BB | CD8 | none |
| ASP | CD137/41BB | CD4 | none |
| ASP | CD137/41BB | b2c | none |
| ASP | CD137/41BB | CD137/41BB | none |
| ASP | CD137/41BB | ICOS | none |
| ASP | CD137/41BB | CD27 | none |
| ASP | CD137/41BB | CD28δ | none |
| ASP | CD137/41BB | CD80 | none |
| ASP | CD137/41BB | CD86 | none |
| ASP | CD137/41BB | OX40 | none |
| ASP | CD137/41BB | DAP10 | none |
| ASP | CD137/41BB | MyD88 | none |
| ASP | CD137/41BB | CD7 | none |
| ASP | CD137/41BB | DAP12 | none |
| ASP | CD137/41BB | MyD88 | none |
| ASP | CD137/41BB | CD7 | none |
| ASP | ICOS | CD28 | none |
| ASP | ICOS | CD8 | none |
| ASP | ICOS | CD4 | none |
| ASP | ICOS | b2c | none |
| ASP | ICOS | CD137/41BB | none |
| ASP | ICOS | ICOS | none |
| ASP | ICOS | CD27 | none |
| ASP | ICOS | CD28δ | none |
| ASP | ICOS | CD80 | none |
| ASP | ICOS | CD86 | none |
| ASP | ICOS | OX40 | none |
| ASP | ICOS | DAP10 | none |
| ASP | ICOS | MyD88 | none |
| ASP | ICOS | CD7 | none |
| ASP | ICOS | DAP12 | none |
| ASP | ICOS | MyD88 | none |
| ASP | ICOS | CD7 | none |
| ASP | ICOS | CD28 | none |
| ASP | ICOS | CD8 | none |
| ASP | ICOS | CD4 | none |
| ASP | ICOS | b2c | none |
| ASP | ICOS | CD137/41BB | none |
| ASP | ICOS | ICOS | none |
| ASP | ICOS | CD27 | none |
| ASP | ICOS | CD28δ | none |
| ASP | ICOS | CD80 | none |
| ASP | ICOS | CD86 | none |
| ASP | ICOS | OX40 | none |
| ASP | ICOS | DAP10 | none |
| ASP | ICOS | MyD88 | none |
| ASP | ICOS | CD7 | none |
| ASP | ICOS | DAP12 | none |
| ASP | ICOS | MyD88 | none |
| ASP | ICOS | CD7 | none |
| ASP | CD27 | CD28 | none |
| ASP | CD27 | CD8 | none |
| ASP | CD27 | CD4 | none |
| ASP | CD27 | b2c | none |
| ASP | CD27 | CD137/41BB | none |
| ASP | CD27 | ICOS | none |
| ASP | CD27 | CD27 | none |
| ASP | CD27 | CD28δ | none |
| ASP | CD27 | CD80 | none |
| ASP | CD27 | CD86 | none |
| ASP | CD27 | OX40 | none |
| ASP | CD27 | DAP10 | none |
| ASP | CD27 | MyD88 | none |
| ASP | CD27 | CD7 | none |
| ASP | CD27 | DAP12 | none |
| ASP | CD27 | MyD88 | none |
| ASP | CD27 | CD7 | none |
| ASP | CD28δ | CD28 | none |
| ASP | CD28δ | CD8 | none |
| ASP | CD28δ | CD4 | none |
| ASP | CD28δ | b2c | none |
| ASP | CD28δ | CD137/41BB | none |
| ASP | CD28δ | ICOS | none |
| ASP | CD28δ | CD27 | none |
| ASP | CD28δ | CD28δ | none |
| ASP | CD28δ | CD80 | none |
| ASP | CD28δ | CD86 | none |
| ASP | CD28δ | OX40 | none |
| ASP | CD28δ | DAP10 | none |
| ASP | CD28δ | MyD88 | none |
| ASP | CD28δ | CD7 | none |
| ASP | CD28δ | DAP12 | none |
| ASP | CD28δ | MyD88 | none |
| ASP | CD28δ | CD7 | none |
| ASP | CD80 | CD28 | none |
| ASP | CD80 | CD8 | none |
| ASP | CD80 | CD4 | none |
| ASP | CD80 | b2c | none |
| ASP | CD80 | CD137/41BB | none |
| ASP | CD80 | ICOS | none |
| ASP | CD80 | CD27 | none |
| ASP | CD80 | CD28δ | none |
| ASP | CD80 | CD80 | none |
| ASP | CD80 | CD86 | none |
| ASP | CD80 | OX40 | none |
| ASP | CD80 | DAP10 | none |
| ASP | CD80 | MyD88 | none |
| ASP | CD80 | CD7 | none |
| ASP | CD80 | DAP12 | none |
| ASP | CD80 | MyD88 | none |
| ASP | CD80 | CD7 | none |
| ASP | CD86 | CD28 | none |
| ASP | CD86 | CD8 | none |
| ASP | CD86 | CD4 | none |
| ASP | CD86 | b2c | none |
| ASP | CD86 | CD137/41BB | none |
| ASP | CD86 | ICOS | none |
| ASP | CD86 | CD27 | none |
| ASP | CD86 | CD28δ | none |
| ASP | CD86 | CD80 | none |
| ASP | CD86 | CD86 | none |
| ASP | CD86 | OX40 | none |
| ASP | CD86 | DAP10 | none |
| ASP | CD86 | MyD88 | none |
| ASP | CD86 | CD7 | none |
| ASP | CD86 | DAP12 | none |
| ASP | CD86 | MyD88 | none |
| ASP | CD86 | CD7 | none |
| ASP | OX40 | CD28 | none |
| ASP | OX40 | CD8 | none |
| ASP | OX40 | CD4 | none |
| ASP | OX40 | b2c | none |
| ASP | OX40 | CD137/41BB | none |
| ASP | OX40 | ICOS | none |
| ASP | OX40 | CD27 | none |
| ASP | OX40 | CD28δ | none |
| ASP | OX40 | CD80 | none |
| ASP | OX40 | CD86 | none |
| ASP | OX40 | OX40 | none |
| ASP | OX40 | DAP10 | none |
| ASP | OX40 | MyD88 | none |
| ASP | OX40 | CD7 | none |
| ASP | OX40 | DAP12 | none |
| ASP | OX40 | MyD88 | none |
| ASP | OX40 | CD7 | none |
| ASP | DAP10 | CD28 | none |
| ASP | DAP10 | CD8 | none |
| ASP | DAP10 | CD4 | none |
| ASP | DAP10 | b2c | none |
| ASP | DAP10 | CD137/41BB | none |
| ASP | DAP10 | ICOS | none |
| ASP | DAP10 | CD27 | none |
| ASP | DAP10 | CD28δ | none |
| ASP | DAP10 | CD80 | none |
| ASP | DAP10 | CD86 | none |
| ASP | DAP10 | OX40 | none |
| ASP | DAP10 | DAP10 | none |
| ASP | DAP10 | MyD88 | none |
| ASP | DAP10 | CD7 | none |
| ASP | DAP10 | DAP12 | none |
| ASP | DAP10 | MyD88 | none |
| ASP | DAP10 | CD7 | none |
| ASP | DAP12 | CD28 | none |
| ASP | DAP12 | CD8 | none |
| ASP | DAP12 | CD4 | none |
| ASP | DAP12 | b2c | none |
| ASP | DAP12 | CD137/41BB | none |
| ASP | DAP12 | ICOS | none |
| ASP | DAP12 | CD27 | none |
| ASP | DAP12 | CD28δ | none |
| ASP | DAP12 | CD80 | none |
| ASP | DAP12 | CD86 | none |
| ASP | DAP12 | OX40 | none |
| ASP | DAP12 | DAP10 | none |
| ASP | DAP12 | MyD88 | none |
| ASP | DAP12 | CD7 | none |
| ASP | DAP12 | DAP12 | none |
| ASP | DAP12 | MyD88 | none |
| ASP | DAP12 | CD7 | none |
| ASP | MyD88 | CD28 | none |
| ASP | MyD88 | CD8 | none |
| ASP | MyD88 | CD4 | none |
| ASP | MyD88 | b2c | none |
| ASP | MyD88 | CD137/41BB | none |
| ASP | MyD88 | ICOS | none |
| ASP | MyD88 | CD27 | none |
| ASP | MyD88 | CD28δ | none |
| ASP | MyD88 | CD80 | none |
| ASP | MyD88 | CD86 | none |
| ASP | MyD88 | OX40 | none |
| ASP | MyD88 | DAP10 | none |
| ASP | MyD88 | MyD88 | none |
| ASP | MyD88 | CD7 | none |
| ASP | MyD88 | DAP12 | none |
| ASP | MyD88 | MyD88 | none |
| ASP | MyD88 | CD7 | none |
| ASP | CD7 | CD28 | none |
| ASP | CD7 | CD8 | none |
| ASP | CD7 | CD4 | none |
| ASP | CD7 | b2c | none |
| ASP | CD7 | CD137/41BB | none |
| ASP | CD7 | ICOS | none |
| ASP | CD7 | CD27 | none |
| ASP | CD7 | CD28δ | none |
| ASP | CD7 | CD80 | none |
| ASP | CD7 | CD86 | none |
| ASP | CD7 | OX40 | none |
| ASP | CD7 | DAP10 | none |
| ASP | CD7 | MyD88 | none |
| ASP | CD7 | CD7 | none |
| ASP | CD7 | DAP12 | none |
| ASP | CD7 | MyD88 | none |
| ASP | CD7 | CD7 | none |
| ASP | BTNL3 | CD28 | none |
| ASP | BTNL3 | CD8 | none |
| ASP | BTNL3 | CD4 | none |
| ASP | BTNL3 | b2c | none |
| ASP | BTNL3 | CD137/41BB | none |
| ASP | BTNL3 | ICOS | none |
| ASP | BTNL3 | CD27 | none |
| ASP | BTNL3 | CD28δ | none |
| ASP | BTNL3 | CD80 | none |
| ASP | BTNL3 | CD86 | none |
| ASP | BTNL3 | OX40 | none |
| ASP | BTNL3 | DAP10 | none |
| ASP | BTNL3 | MyD88 | none |
| ASP | BTNL3 | CD7 | none |
| ASP | BTNL3 | DAP12 | none |
| ASP | BTNL3 | MyD88 | none |
| ASP | BTNL3 | CD7 | none |
| ASP | NKG2D | CD28 | none |
| ASP | NKG2D | CD8 | none |
| ASP | NKG2D | CD4 | none |
| ASP | NKG2D | b2c | none |
| ASP | NKG2D | CD137/41BB | none |
| ASP | NKG2D | ICOS | none |
| ASP | NKG2D | CD27 | none |
| ASP | NKG2D | CD28δ | none |
| ASP | NKG2D | CD80 | none |
| ASP | NKG2D | CD86 | none |
| ASP | NKG2D | OX40 | none |
| ASP | NKG2D | DAP10 | none |
| ASP | NKG2D | MyD88 | none |
| ASP | NKG2D | CD7 | none |
| ASP | NKG2D | DAP12 | none |
| ASP | NKG2D | MyD88 | none |
| ASP | NKG2D | CD7 | none |

In some embodiments, the anti-ASP binding agent is single chain variable fragment (scFv) antibody. Preferably, such an anti-ASP antigen binding agent is a single chain variable fragment (scFv) anti-GalF antibody, more preferably and anti-GalF4 antibody. The affinity/specificity of an anti-ASP scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the anti-ASP binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence. Preferably, the antibody is any of the monoclonal antibodies EB-A1, EB-A2, EB-A3, EB-A4, EB-A5, EB-A6, and EB-A7 as described by Stynen et al., Infect. Immun. 60(6):2237-2245 (1992).

Also disclosed are bi-specific CARs that target a ASP antigen such as GalF4, and at least one additional disease-associated antigen. Also disclosed are CARs designed to work only in conjunction with another CAR that binds a different antigen, such as another ASP antigen. For example, in these embodiments, the endodomain of the disclosed CAR can contain only an signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### CAR Ligand-Binding Domains

The extracellular domain of the CARs disclosed herein generally comprise an antigen recognition domain that binds a target antigen. Such antigen-specific binding domains are typically derived from an antibody. In some embodiments, the antigen-binding domain is a functional antibody fragment or derivative thereof (e.g., an scFv or a Fab, or any suitable antigen binding fragment of an antibody). In preferred embodiments, the antigen-binding domain is a single-chain variable fragment (scFv). In some such embodiments, the scFv is from a monoclonal antibody (mAb). In certain preferred embodiments, the antigen-specific binding domain (e.g., the scFv) is fused to the transmembrane and/or signaling motifs involved in lymphocyte activation as disclosed in Sadelain et al., Nat. Rev. Cancer 3:35-45 (2003), incorporated herein by reference in its entirety.

### Anti-ASP Antibodies and scFvs

In some embodiments, the anti-ASP scFv employed in the CARs of the present invention can comprise a variable heavy (V_{H}) domain having CDR1, CDR2 and CDR3 sequences and a variable light (V_{L}) domain having CDR1, CDR2 and CDR3 sequences. In preferred embodiments, the scFv is an anti-GalF4 scFv.

Some such antibodies from which scFvs may be derived and employed in the CARs of the present invention include, for example, monoclonal antibodies EB-A1, EB-A2, EB-A3, EB-A4, EB-A5, EB-A6, and EB-A7 as described by Stynen et al., Infect. Immun. 60(6):2237-2245 (1992) (incorporated herein by reference in its entirety), all shown to recognize and bind to the galactofuranose-containing side chains of the Aspergillus cell wall galactomannan molecule. See also Latge et al., Medical Mycology 47(Supplement 1):S104-S109 (2009), also incorporated by reference in its entirety.

### Nucleic Acids and Vectors

Also disclosed are polynucleotides and polynucleotide vectors encoding the disclosed ASP-specific CARs that allow expression of the ASP-specific CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells”). In some embodiments, these cells are obtained from the subject to be treated (i.e. are autologous). However, in some preferred embodiments, immune effector cell lines or donor effector cells that are allogeneic to the patient are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes, preferably cytotoxic T lymphocytes (CTLs).

In some embodiments, the immune effector cells that comprise a CAR as described herein are pluripotent stem cells that are capable of differentiating into a cell of the immune system, for example, a cytotoxic T cell. In a preferred embodiment, the immune effector cells of the present invention are CAR-expressing induced pluripotent stem cells (iPSCs).

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described — naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells and constitute a critical cellular subset of the innate immune system (Godfrey et al., Leuk. Lymphoma 53:1666-1676 (2012)). Unlike cytotoxic CD8⁺ T lymphocytes, NK cells launch cytotoxicity against tumor cells without the requirement for prior sensitization and can also eradicate MHC-I-negative cells. NK cells are safer effector cells, as they may avoid the potentially lethal complications of cytokine storms, tumor lysis syndrome, and on-target, off-tumor effects. Although NK cells have a well-known role as killers of cancer cells, and NK cell impairment has been extensively documented as crucial for progression of MM, the means by which one might enhance NK cell-mediated anti-MM activity has been largely unexplored prior to the disclosed CARs.

Epstein-Barr virus (EBV)-induced lymphoproliferative diseases (EBV-LPDs) and other EBV-associated cancers are a significant cause of morbidity and mortality for recipients of allogeneic hematopoietic cell transplantation (HCT) or solid organ transplants (SOT), particularly in those who have received certain T-cell reactive Abs to prevent or treat GVHD. Prophylaxis and treatment by the adoptive transfer of autologous or allogeneic EBV-specific cytotoxic T cells and the subsequent long-term restoration of immunity against EBV-associated lymphoproliferation have provided positive outcomes in the management of these uniformly fatal complications of allogeneic tissue transfer. Therefore, in some embodiments, the disclosed immune effector cells that comprise one or more of the CAR polypeptides of the present invention are allogeneic or autologous EBV-specific cytotoxic T lymphocytes (CTLs). For example, generation of EBV-specific cytotoxic T cells may involve isolating PBMCs from of an EBV-seropositive autologous or allogenic donor and enriching them for T cells by depletion of monocytes and NK cells. EBV-specific cytotoxic T cells may also be produced by contacting donor PBMCs or purified donor T cells with a “stimulator” cell that expresses one or more EBV antigen(s) and presents the EBV antigen(s) to unstimulated T cells, thereby causing stimulation and expansion of EBV-specific CTLs. Notably, in some preferred embodiments, such methods comprise obtaining a sample of cells (e.g., PBMC) from a subject comprising CD3⁺ cells and contacting said CD3⁺ cells with antigen and/or antigen-presenting stimulator cells. Preferably, the CD3⁺ T cells are isolated from the sample prior to contacting the antigen by methods known in the art (e.g., positive selection of CD3⁺ cells from the sample and/or negative selection by depletion of undesired cells or components from the sample). For example, and without limitation, such methods include selection using fluorescence activated cell sorting (FACS), with anti-CD3 beads (e.g., magnetic beads), plastic adherence, depletion of NK cells using anti-CD56, elutriation, and/or combinations thereof. EBV antigens include, for example, latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. Cytotoxic T cells that comprise T cell receptor(s) which recognize one or more EBV-specific antigens are deemed to have been “sensitized” to those EBV antigen(s) and are therefore termed “EBV-sensitized cytotoxic T cells” herein. Known methods for generating allogeneic or autologous EBV-specific cytotoxic T cell populations that may comprise one or more of the CAR polypeptides of the present invention are described, for example, in Barker et al., Blood 116(23):5045-49 (2010); Doubrovina et al., Blood 119(11):2644-56 (2012); Koehne et al., Blood 99(5):1730-40 (2002); and Smith et al., Cancer Res. 72(5):1116-25 (2012), which are incorporated by reference for these teachings. Similarly, cytotoxic T cells may be “sensitized” to other viral antigens, including cytomegalovirus (CMV), papillomavirus (e.g., HPV), adenovirus, polyomavirus (e.g., BKV, JCV, and Merkel cell virus), retrovirus (e.g., HTLV-I, also including lentivirus such as HIV), picomavirus (e.g., Hepatitis A virus), hepadnavirus (e.g., Hepatitis B virus), hepacivirus (e.g., Hepatitis C virus), deltavirus (e.g., Hepatitis D virus), hepevirus (e.g., Hepatitis E virus), and the like. In some preferred embodiments, the target antigen is from an oncovirus. In some such embodiments, the T cells used for generating the CAR-T cells of the invention are polyfunctional T-cells, i.e., those T cells that are capable of inducing multiple immune effector functions, that provide a more effective immune response to a pathogen than do cells that produce, for example, only a single immune effector (e.g. a single biomarker such as a cytokine or CD107a). Less-polyfunctional, monofunctional, or even “exhausted” T cells may dominate immune responses during chronic infections, thus negatively impacting protection against virus-associated complications. In further preferred embodiments, the CAR-T cells of the invention are polyfunctional. In certain embodiments, at least 50% of the T cells used for generating the CAR-T cells of the invention are CD4+ T cells. In some such embodiments, said T cells are less than 50% CD4+ T cells. In still further embodiments, said T cells are predominantly CD4+ T cells. In some embodiments, at least 50% of the T cells used for generating the CAR-T cells of the invention are CD8+ T cells. In some such embodiments, said T cells are less than 50% CD8+ T cells. In still further embodiments, said T cells are predominantly CD8+ T cells. In some embodiments, the T cells (e.g., the sensitized T cells and/or CAR-T cells described herein) are stored in a cell library or bank before they are administered to the subject.

### Therapeutic Methods

Immune effector cells expressing the disclosed CARs can elicit an anti-fungal immune response against ASP-expressing targets. The anti-fungal immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to ASP.

Immune effector cells expressing the disclosed CARs may find use for the prevention and/or treatment of a wide variety of diseases or disorders that are associated with Aspergillus infection. Such diseases and disorders include, for example, pulmonary aspergillosis, allergic bronchopulmonary aspergillosis, aspergilloma, chronic pulmonary aspergilloma, severe asthma with Aspergillus sensitization, chronic cavitary pulmonary aspergillosis, and chronic fibrosing pulmonary aspergillosis.

Adoptive transfer of immune effector cells expressing chimeric antigen receptors is a promising anti-fungal therapeutic. Following the collection of a patient’s immune effector cells, the cells may be genetically engineered to express the disclosed ASP-specific CARs, then infused back into the patient (i.e., autologous cell transfer). Moreover, immune effector cells obtained from a donor other than the patient may be genetically engineered to express the disclosed ASP-specific CARs, then the CAR-containing cells infused into the patient (i.e., allogeneic cell transfer). In one specific embodiment, the immune effector cells which comprise an anti-ASP CAR polypeptide are allogeneic EBV-specific cytotoxic T cells.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat an Aspergillus-associated disease, i.e., intrapleurally, and the like. The quantity and frequency of administration will be determined by such factors as the condition of the patient, and the severity of the patient’s disease, although appropriate dosages may be determined by clinical trials.

When “an immunologically effective amount” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676 (1988)). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, intrapleurally, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In some embodiments, the disclosed CARs are administered in combination with surgery. The disclosed CARs may also be administered in combination with anti-mycotic (anti-fungal) medications. Included without limitation are those drugs known in the art as standard treatment for aspergillosis, such as corticosteroids, itraconazole, voriconazole lipid amphotericin formulations, posaconazole, isavuconazole, itraconazole, caspofungin, micafungin, and amphotericin B.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/ tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against an ASP related antigen and bound to an intracellular costimulatory and stimulatory domain. The second extracellular antigen binding domain however is specific for normal tissue and bound to an intracellular inhibitory domain. Incorporation of multiple intracellular inhibitory domains to the iCAR is also possible. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for infected/ invaded tissue and is advantageous in situations where certain normal tissues may express very low levels of an ASP-associated antigen that would lead to off target effects with a standard CAR (Morgan 2010). A conditional CAR-T cell expresses an extracellular antigen binding domain connected to an intracellular costimulatory domain and a separate, intracellular co-stimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional- CAR design that also releases cytokines like a TRUCK. In some embodiments, a dual-conditional CAR-T cell could be made such that it expresses two CARs with two separate antigen binding domains against two distinct ASP antigens, each bound to their respective costimulatory domains. The costimulatory domain would only become functional with the stimulatory domain after the activating molecule is administered. For this CAR-T cell to be effective the ASP must express both ASP antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the cells transduced with CARs may be autologous or allogeneic to a patient to which they are administered.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

### Exemplification

### Example 1: ASP-CAR Design

Cytotoxic T-cells (CTLS), sensitized to EBV antigen, are used to engineer CAR T cells that selectively target at least one Aspergillus-associated antigenic epitope. Preferably, the Aspergillus-associated antigenic epitope is an epitope expressed in the cell wall of Aspergillus fumigatus such as, for example, a galactofuranose-containing antigenic epitope, more preferably, a GalF4 antigenic epitope. The CAR polypeptide is specifically designed to reduce CAR T cell exhaustion and enhance CAR T cell persistence in the subject. CAR signaling domains are optimized through a combination of co-stimulatory domains (i.e., CD28 and mutants/variants thereof) and signal domain mutants (i.e., CD3ζ lacking one or more functional ITAM domains, preferably lacking functionality in the two C-terminal ITAM domains, i.e., ITAM2 and ITAM3).

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A chimeric antigen receptor (CAR) polypeptide, comprising an Aspergillus (ASP) antigen-binding domain that binds to an ASP antigen; a transmembrane domain; an intracellular signaling domain; and at least one co-stimulatory signaling region.

2-3. (canceled)

4. The CAR polypeptide of claim 1, wherein the ASP antigen is a galactofuranose-containing antigen, such as GalF4.

5. (canceled)

6. The CAR polypeptide of claim 1, wherein the ASP antigen-binding domain is an anti-galactofuranose scFv.

7. (canceled)

8. The CAR polypeptide of claim 1, wherein the transmembrane domain comprises the transmembrane domain of CD28 and/or 41BB.

9. The CAR polypeptide of claim 1, wherein the intracellular signaling domain comprises at least one signaling domain of any one of the polypeptides CD8, CD3ζ, CD3δ, CD3γ, CD3ε, CD32, DAP10, DAP12, CD79a, CD79b, CD28, CD3C, CD4, b2c, 41BB, ICOS, CD27, CD28δ, CD80, NKp30, OX40, FcγRI-γ, FcγRIII-γ, FcεRI-β, Fcε,RI-γ, mutants thereof, or any combinations thereof.

10. The CAR polypeptide of claim 1, wherein the at least one co-stimulatory signaling region comprises a signaling domain of any one of the polypeptides CD8, CD3ζ, CD3δ, CD3γ, CD3ε, CD32, DAP10, DAP12, CD79a, CD79b, CD28, CD3C, CD4, b2c, 41BB, ICOS, CD27, CD28δ, CD80, NKp30, OX40, FcγRI-γ, FcγRIII-γ, FcεRI-β, FcεRI-γ, mutants thereof, or any combinations thereof.

11. The CAR polypeptide of claim 1, wherein the at least one co-stimulatory signaling region comprises a signaling domain of CD28 or a mutant thereof.

12-15. (canceled)

16. The CAR polypeptide of claim 9, wherein at least one signaling domain of the intracellular signaling domain comprises a native CD3ζ, or a mutant thereof.

17-19. (canceled)

20. The CAR polypeptide of claim 1, further comprising a hinge sequence, wherein the hinge sequence is derived from a CD8a molecule or a CD28 molecule.

21. (canceled)

22. A nucleic acid encoding the CAR polypeptide of claim 1.

23. A vector comprising the nucleic acid of claim 22.

24-25. (canceled)

26. A cell expressing the CAR polypeptide of claim 1, wherein the cell is an αβT cell, γδT cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, or a regulatory T cell.

27-31. (canceled)

32. The cell of claim 26, wherein the cell is a viral antigen-sensitized CTL, such as an EBV-sensitized CTL.

33-34. (canceled)

35. The cell of claim 26, wherein the cell exhibits an anti-fungal effect when the antigen binding domain of the chimeric antigen receptor polypeptide binds to ASP.

36. The cell of claim 26, which is allogeneic to a human patient to which it is administered, or is autologous to a human patient to which it is administered.

37-39. (canceled)

40. A method of treating an ASP-associated disease or disorder in a mammal in need thereof, the method comprising administering to the mammal an effective amount of the CAR-expressing cells of claim 26, thereby effectively treating said disease or disorder in said mammal.

41. The method of claim 40, wherein the ASP-associated disease or disorder is pulmonary aspergillosis, allergic bronchopulmonary aspergillosis, aspergilloma, chronic pulmonary aspergilloma, severe asthma with Aspergillus sensitization, chronic cavitary pulmonary aspergillosis, or chronic fibrosing pulmonary aspergillosis.

42. The method of claim 40, wherein the CAR-expressing cells administered to the mammal are autologous to said mammal, or wherein the CAR-expressing cells administered to the mammal are allogeneic to said mammal.

43. (canceled)

44. The method of claim 40, wherein the mammal is human.
